(19) United States
(12) Patent Application Publication
(10) Pub. No.: US 2022/0332782 A1
(43) Pub. Date: Oct. 20, 2022

(54) Combined Artificial Cell Death/Reporter System Polypeptide for Chimeric Antigen Receptor Cell and Uses Thereof

(71) Applicant: Century Therapeutics, Inc., Philadelphia, PA (US)

(72) Inventors: Michael Francis Naso, Philadelphia, PA (US); Luis Borges, Philadelphia, PA (US); Buddha Gurung, Philadelphia, PA (US)

(21) Appl. No.: 17/657,803

(22) Filed: Apr. 4, 2022

Related U.S. Application Data
(60) Provisional application No. 63/171,652, filed on Apr. 7, 2021.

(51) Int. Cl. C07K 14/705; A61P 35/00; C07K 14/035; C12N 5/078

(52) U.S. Cl. CPC C07K 14/705; A61P 35/00; C07K 14/035; C12N 5/0634; C07K 2319/03; C07K 2319/02; C07K 2319/33

(57) ABSTRACT

A combined artificial cell death/reporter system polypeptide containing a herpes simplex virus thymidine kinase (HSV-tk) fused to a prostate-specific membrane antigen (PSMA) polypeptide via a linker is described. Also described are polynucleotides encoding the artificial cell death polypeptide, cells expressing the artificial cell death polypeptide and related methods.

### CROSS-REFERENCE TO RELATED APPLICATIONS

This application claims the benefit of U.S. Provisional Patent Application No. 63/171,652 filed Apr. 7, 2021, which is incorporated by reference herein in its entirety.

### TECHNICAL FIELD

This application provides a combined artificial cell death/reporter polypeptide for use with a CAR engineered immune effector cells for human therapeutics. Also provided are genetically engineered induced pluripotent stem cells (iPSCs) or derivative cells thereof expressing a combined artificial cell death/reporter system polypeptide. Also provided are related vectors, polynucleotides, and pharmaceutical compositions.

### REFERENCE TO SEQUENCE LISTING SUBMITTED ELECTRONICALLY

This application contains a sequence listing, which is submitted electronically via EFS-Web as an ASCII formatted sequence listing with a file name “6US2 Sequence Listing” and a creation date of Mar. 24, 2022 and having a size of 166 kb. The sequence listing submitted via EFS-Web is part of the specification and is herein incorporated by reference in its entirety.

### BACKGROUND

Chimeric antigen receptors (CARs) significantly enhance anti-tumor activity of immune effector cells. CARs are engineered receptors typically comprising an extracellular targeting domain that is linked to a linker peptide, a transmembrane (TM) domain, and one or more intracellular signaling domains. Traditionally, the extracellular domain consists of an antigen binding fragment of an antibody (such as a single chain Fv, scFv) that is specific for a given tumor-associated antigen (TAA) or cell surface target. The extracellular domain confers the tumor specificity of the CAR, while the intracellular signaling domain activates the T cell that has been genetically engineered to express the CAR upon TAA/target engagement. The engineered immune effector cells are re-infused into cancer patients, where they specifically engage and kill cells expressing the TAA target of the CAR (Maus et al., Blood. 2014 Apr. 24; 123(17):2625-35; Curran and Brentjens, J Clin Oncol. 2015 May 20; 33(15):1703-6).

Autologous, patient-specific CAR-T therapy has emerged as a powerful and potentially curative therapy for cancer, especially for CD19-positive hematological malignancies. However, the autologous T cells must be generated on a custom-made basis, which remains a significant limiting factor for large-scale clinical application due to the production costs and the risk of production failure. The development of CAR-T technology and its wider application is also limited due to a number of other key shortcomings, including, e.g., a) an inefficient anti-tumor response in solid tumors, b) limited penetration and susceptibility of adoptively transferred CAR T cells to an immunosuppressive tumor microenvironment (TME), c) poor persistence of CAR-T cells in vivo, d) serious adverse events in the patients including cytokine release syndrome (CRS) and graft-versus-host disease (GVHD) mediated by the CAR-T, and e) the time required for manufacturing.

Since cell therapies, such as CAR-T therapy, have a long or indefinite half-life, and therefore toxicity can be progressive, cells have been engineered to include a safety switch to eliminate the infused cells in case of adverse events. As such, CAR cells have been engineered to include a gene for an artificial cell death polypeptide (a “suicide gene”) which is a genetically encoded molecule that allows selective destruction of the CAR cell allowing selective ablation of the gene modified cells, preventing collateral damage to contiguous cells and/or tissues. The artificial cell death polypeptide could mediate induction of apoptosis, inhibition of protein synthesis, DNA replication, growth arrest, transcriptional and post-transcriptional genetic regulation and/or antibody-mediated depletion. In some instance, the artificial cell death polypeptide is activated by an exogenous molecule, e.g., an antibody, anti-viral drug, or radioisotopic conjugate drugs, that when activated, triggers apoptosis and/or cell death of a therapeutic cell. In one example, the artificial cell death polypeptide comprises a viral enzyme that is recognized by an antiviral drug. In certain embodiments, the viral enzyme is a herpes simplex virus thymidine kinase (HSV-tk) (Bonin et al, Science. 1997 Jun. 13; 276(5319):1719-24).

In glioblastoma patients, neurotoxicities seen with CAR-T administration have been seen with both systemic and intra-thecal administration—but are always associated with cells accessing the CNS. Toxicity can be seen as a result of “on target” toxicities where the binder chosen for the targeting of the glioblastoma cells are not specific for the tumor cells. For example, where EGFR is used as the targeting antigen, EGFR is expressed on some normal cells in the brain (neural stem cells, dopaminergic neurons, Purkinjie cells of cerebellum, pituitary gland, hypothalamus), and is also expressed on many tissues outside the CNS (a known toxicity of EGFR parent antibodies), which presents a risk from cell extravasation. Other antigens may present other risks. “Off target” toxicities can also occur from inflammatory cytokine release or rapid cell expansion. It is therefore advantageous to engineer cells to include a safety switch to eliminate the infused cells in case of these adverse events.

Reporter gene imaging is a component of molecular imaging that can provide noninvasive assessments of endogenous biologic processes in living subjects and that can be performed using different imaging modalities (Brader et al., J Nucl Med. 2013 February; 54(2):167-72). In particular, gene reporter-probe systems currently offer a non-invasive means to monitor gene therapy, to track the movement of cells or the activation of signal transduction pathways, and to study protein-protein interactions and other aspects of signal transduction. Patent application WO 2015/143029 discloses a method for using prostate-specific membrane antigen (PSMA) as an imaging reporter by introducing a reporter gene construct comprising a PSMA gene operably linked to a transcriptional promoter to a cell, allowing the cell to express the PSMA protein and then using an imaging probe that can detect the PSMA protein to detect and track the cell in the body. HSV-tk reporter genes have been used with some success to image both viral gene therapy and CAR-T therapy in the CNS. However, existing HSV-tk PET tracers (e.g. [18F]FHBG) do not readily cross the blood-brain barrier and require tumor-associated blood brain barrier disruption if given intravenously. Non-specific tracer retention in sites with edema may complicate interpretation in some cases (need pre and post cell infusion imaging).

In engineering cell therapies, it is desirable to minimize the number of genetic edits that need to be made to the cells. Thus, there is a need for engineered cell therapies with multiple functionalities that can be engineered with a minimum number of edits.

### BRIEF SUMMARY

In one general aspect, provided is a polynucleotide encoding a combined artificial cell death/reporter system polypeptide. In certain embodiments, the artificial cell death/reporter system polypeptide comprises an intracellular domain having a herpes simplex virus thymidine kinase (HSV-tk) and a linker, a transmembrane region, and an extracellular domain comprising a prostate-specific membrane antigen (PSMA) extracellular domain or fragment thereof.

In certain embodiments, the linker comprises or consists of an amino acid sequence selected from the group consisting of SEQ ID NO: 25 to 56, such as the linker consisting of the amino acid sequence of SEQ ID NO: 48.

In certain embodiments, the linker comprises an autoprotease peptide sequence, such as an autoprotease peptide sequence selected from the group consisting of porcine teschovirus-1 2A

(P2A), thosea asigna virus 2A (T2A), equine rhinitis A virus 2A (E2A), foot-and-mouth disease virus 18 2A (F2A). In a particular embodiment, the autoprotease peptide is a thosea asigna virus 2A (T2A) peptide comprising the amino acid of SEQ ID NO: 75 or 87.

In certain embodiments, the HSV-tk comprises an amino acid sequence having at least 90%, 91%, 92%, 93%, 94%, 95%, 96%, 97%, 98%, 99% or 100% sequence identity to SEQ ID NO: 71 or 89.

In certain embodiments, the artificial cell death/reporter system polypeptide comprises the HSV-tk fused to a truncated variant PSMA polypeptide via the linker.

In certain embodiments, the truncated variant PSMA polypeptide comprises an amino acid sequence having at least 90%, 91%, 92%, 93%, 94%, 95%, 96%, 97%, 98%, 99% or 100% sequence identity to SEQ ID NO: 72.

In certain embodiments, the combined artificial cell death/reporter system polypeptide comprises an amino acid sequence having at least 90%, 91%, 92%, 93%, 94%, 95%, 96%, 97%, 98%, 99% or 100% sequence identity to SEQ ID NO: 73.

In certain embodiments, the combined artificial cell death/reporter system polypeptide comprises an amino acid sequence having at least 90%, 91%, 92%, 93%, 94%, 95%, 96%, 97%, 98%, 99% or 100% sequence identity to SEQ ID NO: 76, 93, or 94.

In certain embodiments, the polynucleotide comprises a polynucleotide sequence having at least 90%, 91%, 92%, 93%, 94%, 95%, 96%, 97%, 98%, 99% or 100% sequence identity to SEQ ID NO: 74, 77, or 95. Also provided is a polynucleotide encoding an artificial cell death/reporter system polypeptide that is combined with an immune checkpoint inhibitor, CD24, to provide the cell with a “don't eat me signal” to escape macrophage-mediated phagocytosis through expression of anti-phagocytic signals. In certain embodiments, the combined artificial cell death/reporter system/CD24 polypeptide comprises a cluster of differentiation 24 (CD24) fused to a prostate-specific membrane antigen (PSMA) extracellular domain or fragment thereof via a peptide linker that can function as the artificial cell death/reporter system.

In certain embodiments, the CD24 comprises an amino acid sequence having at least 90%, 91%, 92%, 93%, 94%, 95%, 96%, 97%, 98%, 99% or 100% sequence identity to SEQ ID NO: 78.

In certain embodiments, the combined artificial cell death/reporter system polypeptide comprises the CD24 fused to a truncated variant PSMA polypeptide via the linker.

In certain embodiments, the truncated variant PSMA polypeptide comprises an amino acid sequence having at least 90%, 91%, 92%, 93%, 94%, 95%, 96%, 97%, 98%, 99% or 100% sequence identity to SEQ ID NO: 72.

In certain embodiments, the linker comprises an autoprotease peptide sequence, such as an autoprotease peptide sequence selected from the group consisting of porcine teschovirus-1 2A

(P2A), thosea asigna virus 2A (T2A), equine rhinitis A virus 2A (E2A), foot-and-mouth disease virus 18 2A (F2A). In a particular embodiment, the autoprotease peptide is a thosea asigna virus 2A (T2A) peptide comprising the amino acid of SEQ ID NO: 75.

In certain embodiments, the combined artificial cell death/reporter system/CD24 polypeptide comprises an amino acid sequence having at least 90%, 91%, 92%, 93%, 94%, 95%, 96%, 97%, 98%, 99% or 100% sequence identity to SEQ ID NO: 79.

In certain embodiments, the polynucleotide comprises a polynucleotide sequence having at least 90%, 91%, 92%, 93%, 94%, 95%, 96%, 97%, 98%, 99% or 100% sequence identity to SEQ ID NO: 80.

In one general aspect, provided is a polynucleotide encoding a combined artificial cell death/reporter system/CD52 polypeptide. In certain embodiments, the artificial cell death/reporter system/CD52 polypeptide comprises a cluster of differentiation 52 (CD52) fused to a herpes simplex virus thymidine kinase (HSV-tk) or fragment thereof via a peptide linker.

In certain embodiments, the CD52 comprises an amino acid sequence having at least 90%, 91%, 92%, 93%, 94%, 95%, 96%, 97%, 98%, 99% or 100% sequence identity to SEQ ID NO: 91.

In certain embodiments, the artificial cell death/reporter system/CD52 polypeptide comprises the CD52 fused to a truncated variant HSV-tk polypeptide via the linker.

In certain embodiments, the linker comprises an autoprotease peptide sequence, such as an autoprotease peptide sequence selected from the group consisting of porcine teschovirus-1 2A

(P2A), thosea asigna virus 2A (T2A), equine rhinitis A virus 2A (E2A), foot-and-mouth disease virus 18 2A (F2A). In a particular embodiment, the autoprotease peptide is a thosea asigna virus 2A (T2A) peptide comprising the amino acid of SEQ ID NO: 75 or 87.

In certain embodiments, the artificial cell death/reporter system/CD52 polypeptide comprises an amino acid sequence having at least 90%, 91%, 92%, 93%, 94%, 95%, 96%, 97%, 98%, 99% or 100% sequence identity to SEQ ID NO: 96 or 97.

In certain embodiments, the artificial cell death/reporter system/CD52 polypeptide is encoded by a polynucleotide sequence having at least 90%, 91%, 92%, 93%, 94%, 95%, 96%, 97%, 98%, 99% or 100% sequence identity to SEQ ID NO: 98.

In certain embodiments, the HSV-tk comprises an amino acid sequence having at least 90%, 91%, 92%, 93%, 94%, 95%, 96%, 97%, 98%, 99% or 100% sequence identity to SEQ ID NO: 71 or 89.

Also provided is a combined artificial cell death/reporter system polypeptide encoded by a polynucleotide according to embodiments of the application.

Also provided is a vector comprising a polynucleotide according to embodiments of the application.

Also provided is a cell, such as an immune cell, an induced pluripotent stem cell (iPSC) or derivative cell thereof comprising a polynucleotide according to embodiment of the application or a vector according to embodiments of the application, wherein the PSMA polypeptide is expressed extracellularly and the HSV-tk is expressed intracellularly.

In certain embodiments, the cell further comprises a polynucleotide encoding a chimeric antigen receptor.

Also provided is a method of eliminating a cell according to embodiments of the application, comprising contacting the cell with one or more agents that bind to the artificial cell death polypeptide to thereby induce death of the cell.

Also provided is a method of producing a cell expressing the artificial cell death/reporter system polypeptide, comprising introducing a polynucleotide according to embodiments of the application or a vector according to embodiments of the application into a cell to thereby produce the cell expressing the artificial cell death/reporter system polypeptide.

### BRIEF DESCRIPTION OF THE DRAWINGS

The foregoing summary, as well as the following detailed description of preferred embodiments of the present application, will be better understood when read in conjunction with the appended drawings. It should be understood, however, that the application is not limited to the precise embodiments shown in the drawings.

FIG. 1 shows a schematic of a plasmid with a CMV early enhancer/chicken β actin (CAG) promoter, a coding sequence of a herpes simplex virus thymidine kinase (HSV-tk), a coding sequence of a Whitlow linker, a coding sequence of a prostate-specific membrane antigen (PSMA), and a SV40 terminator/polyadenylation signal.

FIG. 2 shows a depiction of the herpes simplex virus thymidine kinase (HSV-tk) prostate-specific membrane antigen (PSMA) (HSV-TK-PSMA) fusion protein on a cell surface. The PSMA portion is extracellular while the HSV-tk is located intracellular.

FIGS. 3A-C show transient expression of the PSMA detected using an APC labelled anti-PSMA antibody in induced pluripotent stem cells (iPSCs) by flow cytometry. FIG. 3A shows PSMA expression in iPSCs expressing a wildtype control. FIG. 3B shows PSMA expression in iPSCs expressing the HSV-TK-PSMA fusion protein. FIG. 3C shows comparison of PSMA expression in the iPSCs expressing wildtype (WT) control or HSV-TK-PSMA fusion protein.

FIG. 4 shows a schematic of a plasmid with a CMV early enhancer/chicken β actin (CAG) promoter, a coding sequence of a herpes simplex virus thymidine kinase (HSV-tk), and a SV40 terminator/polyadenylation signal.

FIGS. 5A-5C show ganciclovir killing of induced pluripotent stem cells (iPSCs) expressing the HSV-TK-PSMA fusion. FIG. 5A shows untransfected iPSCs wildtype (WT) control or iPSCs transfected with HSV-TK-PSMA fusion protein (p1499) or HSV-TK alone (p1474) treated with and without ganciclovir for 24 hours. FIG. 5B shows untransfected iPSCs wildtype (WT) control or iPSCs transfected with HSV-TK-PSMA fusion protein (p1499) or

HSV-TK alone (p1474) treated with and without ganciclovir for 48 hours. FIG. 5C is a graph showing quantification of percent cell confluency of WT cells or cells expressing HSV-TK-PSMA fusion protein treated with and without ganciclovir for 24 or 48 hours.

FIGS. 6A-6B show results from cells transfected with a HSV-TK (H168A)-T2A-PSMA transgene. FIG. 6A shows a schematic of HSV-TK (H168A)-T2A-PSMA transgene. FIG. 6B shows wild-type (WT; un-engineered) or HSV-TK (A168H)-T2A-PSMA engineered iPSCs treated with increasing concentrations of ganciclovir for 60 hours. Visual inspection after 60 hours showed near 100% killing of HSV-TK (A168H)-T2A-PSMA cells with 1 uM ganciclovir, whereas un-engineered cells were unaffected.

FIGS. 7A-7B show results of a cell count of control and PSMA+ cells. FIG. 7A shows a count of HSV-TK (A168H)-T2A-PSMA engineered iPSCs that were differentiated into iNK cells (D21) and sorted for PSMA expression. FIG. 7B shows a relative cell count of HSV-TK (A168H)-T2A-PSMA engineered iPSCs that were differentiated into iNK cells (D21) and sorted for PSMA expression.

### DETAILED DESCRIPTION

Various publications, articles and patents are cited or described in the background and throughout the specification; each of these references is herein incorporated by reference in its entirety. Discussion of documents, acts, materials, devices, articles or the like which has been included in the present specification is for the purpose of providing context for the invention. Such discussion is not an admission that any or all of these matters form part of the prior art with respect to any inventions disclosed or claimed.

Unless defined otherwise, all technical and scientific terms used herein have the same meaning as commonly understood to one of ordinary skill in the art to which this application pertains. Otherwise, certain terms used herein have the meanings as set forth in the specification.

It must be noted that as used herein and in the appended claims, the singular forms “a,” “an,” and “the” include plural reference unless the context clearly dictates otherwise.

Unless otherwise stated, any numerical values, such as a concentration or a concentration range described herein, are to be understood as being modified in all instances by the term “about.” Thus, a numerical value typically includes ±10% of the recited value. For example, a concentration of 1 mg/mL includes 0.9 mg/mL to 1.1 mg/mL. Likewise, a concentration range of 1% to 10% (w/v) includes 0.9% (w/v) to 11% (w/v). As used herein, the use of a numerical range expressly includes all possible subranges, all individual numerical values within that range, including integers within such ranges and fractions of the values unless the context clearly indicates otherwise.

Unless otherwise indicated, the term “at least” preceding a series of elements is to be understood to refer to every element in the series. Those skilled in the art will recognize or be able to ascertain using no more than routine experimentation, many equivalents to the specific embodiments of the application described herein. Such equivalents are intended to be encompassed by the application.

As used herein, the terms “comprises,” “comprising,” “includes,” “including,” “has,” “having,” “contains” or “containing,” or any other variation thereof, will be understood to imply the inclusion of a stated integer or group of integers but not the exclusion of any other integer or group of integers and are intended to be non-exclusive or open-ended. For example, a composition, a mixture, a process, a method, an article, or an apparatus that comprises a list of elements is not necessarily limited to only those elements but can include other elements not expressly listed or inherent to such composition, mixture, process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

As used herein, the conjunctive term “and/or” between multiple recited elements is understood as encompassing both individual and combined options. For instance, where two elements are conjoined by “and/or,” a first option refers to the applicability of the first element without the second. A second option refers to the applicability of the second element without the first. A third option refers to the applicability of the first and second elements together. Any one of these options is understood to fall within the meaning, and therefore satisfy the requirement of the term “and/or” as used herein. Concurrent applicability of more than one of the options is also understood to fall within the meaning, and therefore satisfy the requirement of the term “and/or.”

As used herein, the term “consists of” or variations such as “consist of” or “consisting of,” as used throughout the specification and claims, indicate the inclusion of any recited integer or group of integers, but that no additional integer or group of integers can be added to the specified method, structure, or composition.

As used herein, the term “consists essentially of,” or variations such as “consist essentially of” or “consisting essentially of” as used throughout the specification and claims, indicate the inclusion of any recited integer or group of integers, and the optional inclusion of any recited integer or group of integers that do not materially change the basic or novel properties of the specified method, structure or composition. See M.P.E.P. § 2111.03.

As used herein, “subject” means any animal, preferably a mammal, most preferably a human. The term “mammal” as used herein, encompasses any mammal. Examples of mammals include, but are not limited to, cows, horses, sheep, pigs, cats, dogs, mice, rats, rabbits, guinea pigs, monkeys, humans, etc., more preferably a human.

It should also be understood that the terms “about,” “approximately,” “generally,” “substantially,” and like terms, used herein when referring to a dimension or characteristic of a component of the preferred invention, indicate that the described dimension/characteristic is not a strict boundary or parameter and does not exclude minor variations therefrom that are functionally the same or similar, as would be understood by one having ordinary skill in the art. At a minimum, such references that include a numerical parameter would include variations that, using mathematical and industrial principles accepted in the art (e.g., rounding, measurement or other systematic errors, manufacturing tolerances, etc.), would not vary the least significant digit.

The terms “identical” or percent “identity,” in the context of two or more nucleic acids or polypeptide sequences (e.g., CAR polypeptides and the CAR polynucleotides that encode them), refer to two or more sequences or subsequences that are the same or have a specified percentage of amino acid residues or nucleotides that are the same, when compared and aligned for maximum correspondence, as measured using one of the following sequence comparison algorithms or by visual inspection.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Current Protocols in Molecular Biology, F. M. Ausubel et al., eds., Current Protocols, a joint venture between Greene Publishing Associates, Inc. and John Wiley & Sons, Inc., (1995 Supplement) (Ausubel)).

Examples of algorithms that are suitable for determining percent sequence identity and sequence similarity are the BLAST and BLAST 2.0 algorithms, which are described in Altschul et al. (1990) J. Mol. Biol. 215: 403-410 and Altschul et al. (1997) Nucleic Acids Res. 25: 3389-3402, respectively. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are then extended in both directions along each sequence for as far as the cumulative alignment score can be increased.

Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89:10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences or polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions.

As used herein, the term “isolated” means a biological component (such as a nucleic acid, peptide, protein, or cell) has been substantially separated, produced apart from, or purified away from other biological components of the organism in which the component naturally occurs, i.e., other chromosomal and extrachromosomal DNA and RNA, proteins, cells, and tissues. Nucleic acids, peptides, proteins, and cells that have been “isolated” thus include nucleic acids, peptides, proteins, and cells purified by standard purification methods and purification methods described herein. “Isolated” nucleic acids, peptides, proteins, and cells can be part of a composition and still be isolated if the composition is not part of the native environment of the nucleic acid, peptide, protein, or cell. The term also embraces nucleic acids, peptides and proteins prepared by recombinant expression in a host cell as well as chemically synthesized nucleic acids.

As used herein, the term “polynucleotide,” synonymously referred to as “nucleic acid molecule,” “nucleotides” or “nucleic acids,” refers to any polyribonucleotide or polydeoxyribonucleotide, which can be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that can be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications can be made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short nucleic acid chains, often referred to as oligonucleotides.

A “construct” refers to a macromolecule or complex of molecules comprising a polynucleotide to be delivered to a host cell, either in vitro or in vivo. A “vector,” as used herein refers to any nucleic acid construct capable of directing the delivery or transfer of a foreign genetic material to target cells, where it can be replicated and/or expressed. The term “vector” as used herein comprises the construct to be delivered. A vector can be a linear or a circular molecule. A vector can be integrating or non-integrating. The major types of vectors include, but are not limited to, plasmids, episomal vector, viral vectors, cosmids, and artificial chromosomes. Viral vectors include, but are not limited to, adenovirus vector, adeno-associated virus vector, retrovirus vector, lentivirus vector, Sendai virus vector, and the like.

By “integration” it is meant that one or more nucleotides of a construct is stably inserted into the cellular genome, i.e., covalently linked to the nucleic acid sequence within the cell's chromosomal DNA. By “targeted integration” it is meant that the nucleotide(s) of a construct is inserted into the cell's chromosomal or mitochondrial DNA at a pre-selected site or “integration site”. The term “integration” as used herein further refers to a process involving insertion of one or more exogenous sequences or nucleotides of the construct, with or without deletion of an endogenous sequence or nucleotide at the integration site. In the case, where there is a deletion at the insertion site, “integration” can further comprise replacement of the endogenous sequence or a nucleotide that is deleted with the one or more inserted nucleotides.

As used herein, the term “exogenous” is intended to mean that the referenced molecule or the referenced activity is introduced into, or non-native to, the host cell. The molecule can be introduced, for example, by introduction of an encoding nucleic acid into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the cell. The term “endogenous” refers to a referenced molecule or activity that is present in the host cell in its native form. Similarly, the term when used in reference to expression of an encoding nucleic acid refers to expression of an encoding nucleic acid natively contained within the cell and not exogenously introduced.

As used herein, a “gene of interest” or “a polynucleotide sequence of interest” is a DNA sequence that is transcribed into RNA and in some instances translated into a polypeptide in vivo when placed under the control of appropriate regulatory sequences. A gene or polynucleotide of interest can include, but is not limited to, prokaryotic sequences, cDNA from eukaryotic mRNA, genomic DNA sequences from eukaryotic (e.g., mammalian) DNA, and synthetic DNA sequences. For example, a gene of interest may encode an miRNA, an shRNA, a native polypeptide (i.e. a polypeptide found in nature) or fragment thereof; a variant polypeptide (i.e. a mutant of the native polypeptide having less than 100% sequence identity with the native polypeptide) or fragment thereof; an engineered polypeptide or peptide fragment, a therapeutic peptide or polypeptide, an imaging marker, a selectable marker, and the like.

“Operably-linked” refers to the association of nucleic acid sequences on a single nucleic acid fragment so that the function of one is affected by the other. For example, a promoter is operably-linked with a coding sequence or functional RNA when it is capable of affecting the expression of that coding sequence or functional RNA (i.e., the coding sequence or functional RNA is under the transcriptional control of the promoter). Coding sequences can be operably-linked to regulatory sequences in sense or antisense orientation.

The term “expression” as used herein, refers to the biosynthesis of a gene product. The term encompasses the transcription of a gene into RNA. The term also encompasses translation of RNA into one or more polypeptides, and further encompasses all naturally occurring post-transcriptional and post-translational modifications. The expressed CAR can be within the cytoplasm of a host cell, into the extracellular milieu such as the growth medium of a cell culture or anchored to the cell membrane.

As used herein, the terms “peptide,” “polypeptide,” or “protein” can refer to a molecule comprised of amino acids and can be recognized as a protein by those of skill in the art. The conventional one-letter or three-letter code for amino acid residues is used herein. The terms “peptide,” “polypeptide,” and “protein” can be used interchangeably herein to refer to polymers of amino acids of any length. The polymer can be linear or branched, it can comprise modified amino acids, and it can be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids, etc.), as well as other modifications known in the art.

The peptide sequences described herein are written according to the usual convention whereby the N-terminal region of the peptide is on the left and the C-terminal region is on the right. Although isomeric forms of the amino acids are known, it is the L-form of the amino acid that is represented unless otherwise expressly indicated.

As used herein, the term “engineered immune cell” refers to an immune cell, also referred to as an immune effector cell, that has been genetically modified by the addition of exogenous genetic material in the form of DNA or RNA to the total genetic material of the cell.

### I. Induced Pluripotent Stem Cells (IPSCs) and Immune Effector Cells

IPSCs have unlimited self-renewing capacity. Use of iPSCs enables cellular engineering to produce a controlled cell bank of modified cells that can be expanded and differentiated into desired immune effector cells, supplying large amounts of homogeneous allogeneic therapeutic products.

Provided herein are genetically engineered IPSCs and derivative cells thereof. The selected genomic modifications provided herein enhance the therapeutic properties of the derivative cells. The derivative cells are functionally improved and suitable for allogenic off-the-shelf cell therapies following a combination of selective modalities being introduced to the cells at the level of iPSC through genomic engineering. This approach can help to reduce the side effects mediated by CRS/GVHD and prevent long-term autoimmunity while providing excellent efficacy.

As used herein, the term “differentiation” is the process by which an unspecialized (“uncommitted”) or less specialized cell acquires the features of a specialized cell. Specialized cells include, for example, a blood cell or a muscle cell. A differentiated or differentiation-induced cell is one that has taken on a more specialized (“committed”) position within the lineage of a cell. The term “committed”, when applied to the process of differentiation, refers to a cell that has proceeded in the differentiation pathway to a point where, under normal circumstances, it will continue to differentiate into a specific cell type or subset of cell types, and cannot, under normal circumstances, differentiate into a different cell type or revert to a less differentiated cell type. As used herein, the term “pluripotent” refers to the ability of a cell to form all lineages of the body or soma or the embryo proper. For example, embryonic stem cells are a type of pluripotent stem cells that are able to form cells from each of the three germs layers, the ectoderm, the mesoderm, and the endoderm. Pluripotency is a continuum of developmental potencies ranging from the incompletely or partially pluripotent cell (e.g., an epiblast stem cell or EpiSC), which is unable to give rise to a complete organism to the more primitive, more pluripotent cell, which is able to give rise to a complete organism (e.g., an embryonic stem cell).

As used herein, the terms “reprogramming” or “dedifferentiation” refers to a method of increasing the potency of a cell or dedifferentiating the cell to a less differentiated state. For example, a cell that has an increased cell potency has more developmental plasticity (i.e., can differentiate into more cell types) compared to the same cell in the non-reprogrammed state. In other words, a reprogrammed cell is one that is in a less differentiated state than the same cell in a non-reprogrammed state.

As used herein, the term “induced pluripotent stem cells” or, iPSCs, means that the stem cells are produced from differentiated adult, neonatal or fetal cells that have been induced or changed or reprogrammed into cells capable of differentiating into tissues of all three germ or dermal layers: mesoderm, endoderm, and ectoderm. The iPSCs produced do not refer to cells as they are found in nature.

The term “hematopoietic stem and progenitor cells,” “hematopoietic stem cells,” “hematopoietic progenitor cells,” or “hematopoietic precursor cells” or “HPCs” refers to cells which are committed to a hematopoietic lineage but are capable of further hematopoietic differentiation. Hematopoietic stem cells include, for example, multipotent hematopoietic stem cells (hematoblasts), myeloid progenitors, megakaryocyte progenitors, erythrocyte progenitors, and lymphoid progenitors. Hematopoietic stem and progenitor cells (HSCs) are multipotent stem cells that give rise to all the blood cell types including myeloid (monocytes and macrophages, neutrophils, basophils, eosinophils, erythrocytes, megakaryocytes/platelets, dendritic cells), and lymphoid lineages (T cells, B cells, NK cells). As used herein, “CD34+ hematopoietic progenitor cell” refers to an HPC that expresses CD34 on its surface.

As used herein, the term “immune cell” or “immune effector cell” refers to a cell that is involved in an immune response. Immune response includes, for example, the promotion of an immune effector response. Examples of immune cells include T cells, B cells, natural killer (NK) cells, mast cells, and myeloid-derived phagocytes.

As used herein, the terms “T lymphocyte” and “T cell” are used interchangeably and refer to a type of white blood cell that completes maturation in the thymus and that has various roles in the immune system. A T cell can have the roles including, e.g., the identification of specific foreign antigens in the body and the activation and deactivation of other immune cells. A T cell can be any T cell, such as a cultured T cell, e.g., a primary T cell, or a T cell from a cultured T cell line, e.g., Jurkat, SupTl, etc., or a T cell obtained from a mammal. The T cell can be CD3+ cells. The T cell can be any type of T cell and can be of any developmental stage, including but not limited to, CD4+/CD8+ double positive T cells, CD4+ helper T cells (e.g., Th1 and Th2 cells), CD8+ T cells (e.g., cytotoxic T cells), peripheral blood mononuclear cells (PBMCs), peripheral blood leukocytes (PBLs), tumor infiltrating lymphocytes (TILs), memory T cells, naive T cells, regulator T cells, gamma delta T cells (gd T cells), and the like. Additional types of helper T cells include cells such as Th3 (Treg), Th17, Th9, or Tfh cells. Additional types of memory T cells include cells such as central memory T cells (Tcm cells), effector memory T cells (Tern cells and TEMRA cells). The T cell can also refer to a genetically engineered T cell, such as a T cell modified to express a T cell receptor (TCR) or a chimeric antigen receptor (CAR). The T cell can also be differentiated from a stem cell or progenitor cell.

“CD4+ T cells” refers to a subset of T cells that express CD4 on their surface and are associated with cell-mediated immune response. They are characterized by the secretion profiles following stimulation, which may include secretion of cytokines such as IFN-gamma, TNF-alpha, IL2, IL4 and IL10. “CD4” are 55-kD glycoproteins originally defined as differentiation antigens on T-lymphocytes, but also found on other cells including monocytes/macrophages. CD4 antigens are members of the immunoglobulin supergene family and are implicated as associative recognition elements in MHC (major histocompatibility complex) class II-restricted immune responses. On T-lymphocytes they define the helper/inducer subset.

“CD8+ T cells” refers to a subset of T cells which express CD8 on their surface, are MHC class I-restricted, and function as cytotoxic T cells. “CD8” molecules are differentiation antigens found on thymocytes and on cytotoxic and suppressor T-lymphocytes. CD8 antigens are members of the immunoglobulin supergene family and are associative recognition elements in major histocompatibility complex class I-restricted interactions.

As used herein, the term “NK cell” or “Natural Killer cell” refers to a subset of peripheral blood lymphocytes defined by the expression of CD56 and CD45 and the absence of the T cell receptor (TCR chains). The NK cell can also refer to a genetically engineered NK cell, such as a NK cell modified to express a chimeric antigen receptor (CAR). The NK cell can also be differentiated from a stem cell or progenitor cell.

As used herein, the term “genetic imprint” refers to genetic or epigenetic information that contributes to preferential therapeutic attributes in a source cell or an iPSC, and is retainable in the source cell derived iPSCs, and/or the iPSC-derived hematopoietic lineage cells. As used herein, “a source cell” is a non-pluripotent cell that can be used for generating iPSCs through reprogramming, and the source cell derived iPSCs can be further differentiated to specific cell types including any hematopoietic lineage cells. The source cell derived iPSCs, and differentiated cells therefrom are sometimes collectively called “derived” or “derivative” cells depending on the context. For example, derivative effector cells, or derivative NK or “iNK” cells or derivative T or “iT” cells, as used throughout this application are cells differentiated from an iPSC, as compared to their primary counterpart obtained from natural/native sources such as peripheral blood, umbilical cord blood, or other donor tissues. As used herein, the genetic imprint(s) conferring a preferential therapeutic attribute is incorporated into the iPSCs either through reprogramming a selected source cell that is donor-, disease-, or treatment response-specific, or through introducing genetically modified modalities to iPSC using genomic editing.

The induced pluripotent stem cell (iPSC) parental cell lines can be generated from peripheral blood mononuclear cells (PBMCs) or T-cells using any known method for introducing re-programming factors into non-pluripotent cells such as the episomal plasmid-based process as previously described in U.S. Pat. Nos. 8,546,140; 9,644,184; 9,328,332; and 8,765,470, the complete disclosures of which are incorporated herein by reference. The reprogramming factors can be in a form of polynucleotides, and thus are introduced to the non-pluripotent cells by vectors such as a retrovirus, a Sendai virus, an adenovirus, an episome, and a mini-circle. In particular embodiments, the one or more polynucleotides encoding at least one reprogramming factor are introduced by a lentiviral vector. In some embodiments, the one or more polynucleotides introduced by an episomal vector. In various other embodiments, the one or more polynucleotides are introduced by a Sendai viral vector. In some embodiments, the iPSC's are clonal iPSC's or are obtained from a pool of iPSCs and the genome edits are introduced by making one or more targeted integration and/or in/del at one or more selected sites. In another embodiment, the iPSC's are obtained from human T cells having antigen specificity and a reconstituted TCR gene (hereinafter, also refer to as “T-iPS” cells) as described in U.S. Pat. Nos. 9,206,394, and 10,787,642 hereby incorporated by reference into the present application.

According to a particular aspect, the application relates to an induced pluripotent stem cell (iPSC) cell or a derivative cell thereof comprising a polynucleotide encoding a combined artificial cell death/reporter system polypeptide. In certain embodiments, the iPSC or derivative cell thereof further comprises a polynucleotide encoding chimeric antigen receptor.

### II. Chimeric Antigen Receptor (CAR) Expression

As used herein, the term “chimeric antigen receptor” (CAR) refers to a recombinant polypeptide comprising at least an extracellular domain that binds specifically to an antigen or a target, a transmembrane domain and an intracellular signaling domain. Engagement of the extracellular domain of the CAR with the target antigen on the surface of a target cell results in clustering of the CAR and delivers an activation stimulus to the CAR-containing cell. CARs redirect the specificity of immune effector cells and trigger proliferation, cytokine production, phagocytosis and/or production of molecules that can mediate cell death of the target antigen-expressing cell in a major histocompatibility (MHC)-independent manner.

As used herein, the term “signal peptide” refers to a leader sequence at the amino-terminus (N-terminus) of a nascent CAR protein, which co-translationally or post-translationally directs the nascent protein to the endoplasmic reticulum and subsequent surface expression.

As used herein, the term “extracellular antigen binding domain,” “extracellular domain,” or “extracellular ligand binding domain” refers to the part of a CAR that is located outside of the cell membrane and is capable of binding to an antigen, target or ligand.

As used herein, the term “hinge region” or “hinge domain” refers to the part of a CAR that connects two adjacent domains of the CAR protein, i.e., the extracellular domain and the transmembrane domain of the CAR protein.

As used herein, the term “transmembrane domain” refers to the portion of a CAR that extends across the cell membrane and anchors the CAR to cell membrane.

As used herein, the term “intracellular signaling domain,” “cytoplasmic signaling domain,” or “intracellular signaling domain” refers to the part of a CAR that is located inside of the cell membrane and is capable of transducing an effector signal.

As used herein, the term “stimulatory molecule” refers to a molecule expressed by an immune cell (e.g., T cell) that provides the primary cytoplasmic signaling sequence(s) that regulate primary activation of receptors in a stimulatory way for at least some aspect of the immune cell signaling pathway. Stimulatory molecules comprise two distinct classes of cytoplasmic signaling sequence, those that initiate antigen-dependent primary activation (referred to as “primary signaling domains”), and those that act in an antigen-independent manner to provide a secondary of co-stimulatory signal (referred to as “co-stimulatory signaling domains”).

In certain embodiments, the extracellular domain comprises an antigen binding domain and/or an antigen binding fragment. The antigen binding fragment can, for example, be an antibody or antigen binding fragment thereof that specifically binds a tumor antigen. The antigen binding fragments of the application possess desirable functional properties, including but not limited to high-affinity binding to a tumor antigen.

As used herein, the term “antibody” is used in a broad sense and includes immunoglobulin or antibody molecules including human, humanized, composite and chimeric antibodies and antibody fragments that are monoclonal or polyclonal. In general, antibodies are proteins or peptide chains that exhibit binding specificity to a specific antigen. Antibody structures are well known. Immunoglobulins can be assigned to five major classes (i.e., IgA, IgD, IgE, IgG and IgM), depending on the heavy chain constant domain amino acid sequence. IgA and IgG are further sub-classified as the isotypes IgA1, IgA2, IgG1, IgG2, IgG3 and IgG4. Accordingly, the antibodies of the application can be of any of the five major classes or corresponding sub-classes. Preferably, the antibodies of the application are IgG1, IgG2, IgG3 or IgG4. Antibody light chains of vertebrate species can be assigned to one of two clearly distinct types, namely kappa and lambda, based on the amino acid sequences of their constant domains. Accordingly, the antibodies of the application can contain a kappa or lambda light chain constant domain. According to particular embodiments, the antibodies of the application include heavy and/or light chain constant regions from rat or human antibodies. In addition to the heavy and light constant domains, antibodies contain an antigen-binding region that is made up of a light chain variable region and a heavy chain variable region, each of which contains three domains (i.e., complementarity determining regions 1-3; CDR1, CDR2, and CDR3). The light chain variable region domains are alternatively referred to as LCDR1, LCDR2, and LCDR3, and the heavy chain variable region domains are alternatively referred to as HCDR1, HCDR2, and HCDR3.

As used herein, the term an “isolated antibody” refers to an antibody which is substantially free of other antibodies having different antigenic specificities (e.g., an isolated antibody that specifically binds to the specific tumor antigen is substantially free of antibodies that do not bind to the tumor antigen). In addition, an isolated antibody is substantially free of other cellular material and/or chemicals.

As used herein, the term “monoclonal antibody” refers to an antibody obtained from a population of substantially homogeneous antibodies, i.e., the individual antibodies comprising the population are identical except for possible naturally occurring mutations that can be present in minor amounts. The monoclonal antibodies of the application can be made by the hybridoma method, phage display technology, single lymphocyte gene cloning technology, or by recombinant DNA methods. For example, the monoclonal antibodies can be produced by a hybridoma which includes a B cell obtained from a transgenic nonhuman animal, such as a transgenic mouse or rat, having a genome comprising a human heavy chain transgene and a light chain transgene.

As used herein, the term “antigen-binding fragment” refers to an antibody fragment such as, for example, a diabody, a Fab, a Fab′, a F(ab′)₂, an Fv fragment, a disulfide stabilized Fv fragment (dsFv), a (dsFv)₂, a bispecific dsFv (dsFv-dsFv′), a disulfide stabilized diabody (ds diabody), a single-chain antibody molecule (scFv), a single domain antibody (sdAb), a scFv dimer (bivalent diabody), a multispecific antibody formed from a portion of an antibody comprising one or more CDRs, a camelized single domain antibody, a minibody, a nanobody, a domain antibody, a bivalent domain antibody, a light chain variable domain (VL), a variable domain (VHH) of a camelid antibody, or any other antibody fragment that binds to an antigen but does not comprise a complete antibody structure. An antigen-binding fragment is capable of binding to the same antigen to which the parent antibody or a parent antibody fragment binds.

As used herein, the term “single-chain antibody” refers to a conventional single-chain antibody in the field, which comprises a heavy chain variable region and a light chain variable region connected by a short peptide of about 15 to about 20 amino acids (e.g., a linker peptide).

As used herein, the term “single domain antibody” refers to a conventional single domain antibody in the field, which comprises a heavy chain variable region and a heavy chain constant region or which comprises only a heavy chain variable region.

As used herein, the term “human antibody” refers to an antibody produced by a human or an antibody having an amino acid sequence corresponding to an antibody produced by a human made using any technique known in the art. This definition of a human antibody includes intact or full-length antibodies, fragments thereof, and/or antibodies comprising at least one human heavy and/or light chain polypeptide.

As used herein, the term “humanized antibody” refers to a non-human antibody that is modified to increase the sequence homology to that of a human antibody, such that the antigen-binding properties of the antibody are retained, but its antigenicity in the human body is reduced.

As used herein, the term “chimeric antibody” refers to an antibody wherein the amino acid sequence of the immunoglobulin molecule is derived from two or more species. The variable region of both the light and heavy chains often corresponds to the variable region of an antibody derived from one species of mammal (e.g., mouse, rat, rabbit, etc.) having the desired specificity, affinity, and capability, while the constant regions correspond to the sequences of an antibody derived from another species of mammal (e.g., human) to avoid eliciting an immune response in that species.

As used herein, the term “multispecific antibody” refers to an antibody that comprises a plurality of immunoglobulin variable domain sequences, wherein a first immunoglobulin variable domain sequence of the plurality has binding specificity for a first epitope and a second immunoglobulin variable domain sequence of the plurality has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes do not overlap or do not substantially overlap. In an embodiment, the first and second epitopes are on different antigens, e.g., the different proteins (or different subunits of a multimeric protein). In an embodiment, a multispecific antibody comprises a third, fourth, or fifth immunoglobulin variable domain. In an embodiment, a multispecific antibody is a bispecific antibody molecule, a trispecific antibody molecule, or a tetraspecific antibody molecule.

As used herein, the term “bispecific antibody” refers to a multispecific antibody that binds no more than two epitopes or two antigens. A bispecific antibody is characterized by a first immunoglobulin variable domain sequence which has binding specificity for a first epitope and a second immunoglobulin variable domain sequence that has binding specificity for a second epitope. In an embodiment, the first and second epitopes are on the same antigen, e.g., the same protein (or subunit of a multimeric protein). In an embodiment, the first and second epitopes overlap or substantially overlap. In an embodiment, the first and second epitopes are on different antigens, e.g., the different proteins (or different subunits of a multimeric protein). In an embodiment, a bispecific antibody comprises a heavy chain variable domain sequence and a light chain variable domain sequence which have binding specificity for a first epitope and a heavy chain variable domain sequence and a light chain variable domain sequence which have binding specificity for a second epitope. In an embodiment, a bispecific antibody comprises a half antibody, or fragment thereof, having binding specificity for a first epitope and a half antibody, or fragment thereof, having binding specificity for a second epitope. In an embodiment, a bispecific antibody comprises a scFv, or fragment thereof, having binding specificity for a first epitope, and a scFv, or fragment thereof, having binding specificity for a second epitope. In an embodiment, a bispecific antibody comprises a VHH having binding specificity for a first epitope, and a VHH having binding specificity for a second epitope.

As used herein, an antigen binding domain or antigen binding fragment that “specifically binds to a tumor antigen” refers to an antigen binding domain or antigen binding fragment that binds a tumor antigen, with a K_{D} of 1×10⁻⁷ M or less, preferably 1×10⁻⁸M or less, more preferably 5×10⁻⁹M or less, 1×10⁻⁹ M or less, 5×10⁻¹⁰ M or less, or 1×10⁻¹⁰ M or less. The term “KD” refers to the dissociation constant, which is obtained from the ratio of Kd to Ka (i.e., Kd/Ka) and is expressed as a molar concentration (M). KD values for antibodies can be determined using methods in the art in view of the present disclosure. For example, the KD of an antigen binding domain or antigen binding fragment can be determined by using surface plasmon resonance, such as by using a biosensor system, e.g., a Biacore® system, or by using bio-layer interferometry technology, such as an Octet RED96 system.

The smaller the value of the KD of an antigen binding domain or antigen binding fragment, the higher affinity that the antigen binding domain or antigen binding fragment binds to a target antigen.

In various embodiments, antibodies or antibody fragments suitable for use in the CAR of the present disclosure include, but are not limited to, monoclonal antibodies, bispecific antibodies, multispecific antibodies, chimeric antibodies, polypeptide-Fc fusions, single-chain Fvs (scFv), single chain antibodies, Fab fragments, F(ab′) fragments, disulfide-linked Fvs (sdFv), masked antibodies (e.g., Probodies®), Small Modular ImmunoPharmaceuticals (“SMIPs™”), intrabodies, minibodies, single domain antibody variable domains, nanobodies, VHHs, diabodies, tandem diabodies (TandAb®), anti-idiotypic (anti-Id) antibodies (including, e.g., anti-Id antibodies to antigen-specific TCR), and epitope-binding fragments of any of the above. Antibodies and/or antibody fragments can be derived from murine antibodies, rabbit antibodies, human antibodies, fully humanized antibodies, camelid antibody variable domains and humanized versions, shark antibody variable domains and humanized versions, and camelized antibody variable domains.

In some embodiments, the antigen-binding fragment is an Fab fragment, an Fab′ fragment, an F(ab′)2 fragment, an scFv fragment, an Fv fragment, a dsFv diabody, a VHH, a VNAR, a single-domain antibody (sdAb) or nanobody, a dAb fragment, a Fd′ fragment, a Fd fragment, a heavy chain variable region, an isolated complementarity determining region (CDR), a diabody, a triabody, or a decabody. In some embodiments, the antigen-binding fragment is an scFv fragment.

In certain embodiments, the antigen binding domain of the CAR is a single-domain antibody (sdAb), also known as a nanobody, an antibody fragment consisting of a single monomeric variable antibody domain, including heavy-chain antibodies found in camelids; the so called VHH fragments. (Hamers-Casterman et al., Nature, 363, 446448 (1993); see also U.S. Pat. Nos. 5,759,808; 5,800,988; 5,840,526; and 5,874,541, hereby incorporated by reference). Cartilaginous fishes also have heavy-chain antibodies (IgNAR, ‘immunoglobulin new antigen receptor’), from which single-domain antibodies called VNAR fragments can be obtained, and these can be used in the invention. An alternative approach is to split the dimeric variable domains from common immunoglobulin G (IgG) from humans or mice into monomers. Although most research into single-domain antibodies is currently based on heavy chain variable domains, nanobodies derived from light chains have also been shown to bind specifically to target epitopes and can also be employed.

Alternative scaffolds to immunoglobulin domains that exhibit similar functional characteristics, such as high-affinity and specific binding of target biomolecules, can also be used in the CARs of the present disclosure. Such scaffolds have been shown to yield molecules with improved characteristics, such as greater stability or reduced immunogenicity. Non-limiting examples of alternative scaffolds that can be used in the CAR of the present disclosure include engineered, tenascin-derived, tenascin type III domain (e.g., Centyrin™); engineered, gamma-B crystallin-derived scaffold or engineered, ubiquitin-derived scaffold (e.g., Affilins); engineered, fibronectin-derived, 10th fibronectin type III (10Fn3) domain (e.g., monobodies, AdNectins™ or AdNexins™); engineered, ankyrin repeat motif containing polypeptide (e.g., DARPins™); engineered, low-density-lipoprotein-receptor-derived, A domain (LDLR-A) (e.g., Avimers™); lipocalin (e.g., anticalins); engineered, protease inhibitor-derived, Kunitz domain (e.g., EETI-II/AGRP, BPTI/LACI-D1/ITI-D2); engineered, Protein-A-derived, Z domain (Affibodies™); Sac7d-derived polypeptides (e.g., Nanoffitins® or affitins); engineered, Fyn-derived, SH2 domain (e.g., Fynomers®); CTLD3 (e.g., Tetranectin); thioredoxin (e.g., peptide aptamer); KALBITOR®; the (3-sandwich (e.g., iMab); miniproteins; C-type lectin-like domain scaffolds; engineered antibody mimics; and any genetically manipulated counterparts of the foregoing that retains its binding functionality (Worn A, Pluckthun A, J Mol Biol 305: 989-1010 (2001); Xu L et al., Chem Biol 9: 933-42 (2002); Wikman M et al., Protein Eng Des Sel 17: 455-62 (2004); Binz H et al., Nat Biolechnol 23: 1257-68 (2005); Hey T et al., Trends Biotechnol 23:514-522 (2005); Holliger P, Hudson P, Nat Biotechnol 23: 1126-36 (2005); Gill D, Damle N, Curr Opin Biotech 17: 653-8 (2006); Koide A, Koide S, Methods Mol Biol 352: 95-109 (2007); Skerra, Current Opin. in Biotech., 2007 18: 295-304; Byla P et al., J Biol Chem 285: 12096 (2010); Zoller F et al., Molecules 16: 2467-85 (2011), each of which is incorporated by reference in its entirety).

In some embodiments, the alternative scaffold is Affilin or Centyrin.

In some embodiments, the first polypeptide of the CARs of the present disclosure comprises a leader sequence. The leader sequence can be positioned at the N-terminus the extracellular binding domain. The leader sequence can be optionally cleaved from the extracellular binding domain during cellular processing and localization of the CAR to the cellular membrane. Any of various leader sequences known to one of skill in the art can be used as the leader sequence. Non-limiting examples of peptides from which the leader sequence can be derived include granulocyte-macrophage colony-stimulating factor receptor (GMCSFR), FcεR, human immunoglobulin (IgG) heavy chain (HC) variable region, CD8α, or any of various other proteins secreted by T cells. In various embodiments, the leader sequence is compatible with the secretory pathway of a T cell. In certain embodiments, the leader sequence is derived from human immunoglobulin heavy chain (HC).

In some embodiments, the leader sequence is derived from GMCSFR. In one embodiment, the GMCSFR leader sequence comprises the amino acid sequence set forth in SEQ ID NO: 1, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 1.

In some embodiments, the first polypeptide of the CARs of the present disclosure comprise a transmembrane domain, fused in frame between the extracellular binding domain and the cytoplasmic domain.

The transmembrane domain can be derived from the protein contributing to the extracellular binding domain, the protein contributing the signaling or co-signaling domain, or by a totally different protein. In some instances, the transmembrane domain can be selected or modified by amino acid substitution, deletions, or insertions to minimize interactions with other members of the CAR complex. In some instances, the transmembrane domain can be selected or modified by amino acid substitution, deletions, or insertions to avoid binding of proteins naturally associated with the transmembrane domain. In certain embodiments, the transmembrane domain includes additional amino acids to allow for flexibility and/or optimal distance between the domains connected to the transmembrane domain.

The transmembrane domain can be derived either from a natural or from a synthetic source. Where the source is natural, the domain can be derived from any membrane-bound or transmembrane protein. Non-limiting examples of transmembrane domains of particular use in this disclosure can be derived from (i.e. comprise at least the transmembrane region(s) of) the α, β or ζ chain of the T cell receptor (TCR), CD28, CD3 epsilon, CD45, CD4, CD5, CD8, CD8α, CD9, CD16, CD22, CD33, CD37, CD40, CD64, CD80, CD86, CD134, CD137, or CD154. Alternatively, the transmembrane domain can be synthetic, in which case it will comprise predominantly hydrophobic residues such as leucine and valine. For example, a triplet of phenylalanine, tryptophan and/or valine can be found at each end of a synthetic transmembrane domain.

In some embodiments, it will be desirable to utilize the transmembrane domain of the η or FcεR1γ chains which contain a cysteine residue capable of disulfide bonding, so that the resulting chimeric protein will be able to form disulfide linked dimers with itself, or with unmodified versions of the η or FcεR1γ chains or related proteins. In some instances, the transmembrane domain will be selected or modified by amino acid substitution to avoid binding of such domains to the transmembrane domains of the same or different surface membrane proteins to minimize interactions with other members of the receptor complex. In other cases, it will be desirable to employ the transmembrane domain of η or FcεR1γ and -β, MB1 (Igα.), B29 or CD3-γ, ζ, or η, in order to retain physical association with other members of the receptor complex.

In some embodiments, the transmembrane domain is derived from CD8 or CD28. In one embodiment, the CD8 transmembrane domain comprises the amino acid sequence set forth in SEQ ID NO: 23, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 23. In one embodiment, the CD28 transmembrane domain comprises the amino acid sequence set forth in SEQ ID NO: 24, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 24.

In some embodiments, the first polypeptide of the CAR of the present disclosure comprises a spacer region between the extracellular binding domain and the transmembrane domain, wherein the binding domain, linker, and the transmembrane domain are in frame with each other.

The term “spacer region” as used herein generally means any oligo- or polypeptide that functions to link the binding domain to the transmembrane domain. A spacer region can be used to provide more flexibility and accessibility for the binding domain. A spacer region can comprise up to 300 amino acids, preferably 10 to 100 amino acids and most preferably 25 to 50 amino acids. A spacer region can be derived from all or part of naturally occurring molecules, such as from all or part of the extracellular region of CD8, CD4 or CD28, or from all or part of an antibody constant region. Alternatively, the spacer region can be a synthetic sequence that corresponds to a naturally occurring spacer region sequence, or can be an entirely synthetic spacer region sequence. Non-limiting examples of spacer regions which can be used in accordance to the disclosure include a part of human CD8a chain, partial extracellular domain of CD28, FcγRllla receptor, IgG, IgM, IgA, IgD, IgE, an Ig hinge, or functional fragment thereof. In some embodiments, additional linking amino acids are added to the spacer region to ensure that the antigen-binding domain is an optimal distance from the transmembrane domain. In some embodiments, when the spacer is derived from an Ig, the spacer can be mutated to prevent Fc receptor binding.

In some embodiments, the spacer region comprises a hinge domain. The hinge domain can be derived from CD8α, CD28, or an immunoglobulin (IgG). For example, the IgG hinge can be from IgG1, IgG2, IgG3, IgG4, IgM1, IgM2, IgA1, IgA2, IgD, IgE, or a chimera thereof.

In certain embodiments, the hinge domain comprises an immunoglobulin IgG hinge or functional fragment thereof. In certain embodiments, the IgG hinge is from IgG1, IgG2, IgG3, IgG4, IgM1, IgM2, IgA1, IgA2, IgD, IgE, or a chimera thereof. In certain embodiments, the hinge domain comprises the CH1, CH2, CH3 and/or hinge region of the immunoglobulin. In certain embodiments, the hinge domain comprises the core hinge region of the immunoglobulin. The term “core hinge” can be used interchangeably with the term “short hinge” (a.k.a “SH”). Non-limiting examples of suitable hinge domains are the core immunoglobulin hinge regions include EPKSCDKTHTCPPCP (SEQ ID NO: 57) from IgG1, ERKCCVECPPCP (SEQ ID NO: 58) from IgG2, ELKTPLGDTTHTCPRCP(EPKSCDTPPPCPRCP)₃ (SEQ ID NO: 59) from IgG3, and ESKYGPPCPSCP (SEQ ID NO: 60) from IgG4 (see also Wypych et al., JBC 2008 283(23): 16194-16205, which is incorporated herein by reference in its entirety for all purposes). In certain embodiments, the hinge domain is a fragment of the immunoglobulin hinge.

In some embodiments, the hinge domain is derived from CD8 or CD28. In one embodiment, the CD8 hinge domain comprises the amino acid sequence set forth in SEQ ID NO: 21, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 21. In one embodiment, the CD28 hinge domain comprises the amino acid sequence set forth in SEQ ID NO: 22, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 22.

In some embodiments, the transmembrane domain and/or hinge domain is derived from CD8 or CD28. In some embodiments, both the transmembrane domain and hinge domain are derived from CD8. In some embodiments, both the transmembrane domain and hinge domain are derived from CD28.

In certain aspects, the first polypeptide of CARs of the present disclosure comprise a cytoplasmic domain, which comprises at least one intracellular signaling domain. In some embodiments, cytoplasmic domain also comprises one or more co-stimulatory signaling domains.

The cytoplasmic domain is responsible for activation of at least one of the normal effector functions of the host cell (e.g., T cell) in which the CAR has been placed in. The term “effector function” refers to a specialized function of a cell. Effector function of a T cell, for example, can be cytolytic activity or helper activity including the secretion of cytokines. Thus, the term “signaling domain” refers to the portion of a protein which transduces the effector function signal and directs the cell to perform a specialized function. While usually the entire signaling domain is present, in many cases it is not necessary to use the entire chain. To the extent that a truncated portion of the intracellular signaling domain is used, such truncated portion can be used in place of the intact chain as long as it transduces the effector function signal. The term intracellular signaling domain is thus meant to include any truncated portion of the signaling domain sufficient to transduce the effector function signal.

Non-limiting examples of signaling domains which can be used in the CARs of the present disclosure include, e.g., signaling domains derived from DAP10, DAP12, Fc epsilon receptor I γ chain (FCER1G), FcR β, CD3δ, CD3ε, CD3γ, CD3ζ, CD2, CD5, CD22, CD226, CD66d, CD79A, and CD79B.

In some embodiments, the cytoplasmic domain comprises a CD3t signaling domain. In one embodiment, the CD3t signaling domain comprises the amino acid sequence set forth in SEQ ID NO: 6, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 6.

In some embodiments, the cytoplasmic domain further comprises one or more co-stimulatory signaling domains. In some embodiments, the one or more co-stimulatory signaling domains are derived from CD28, 41BB, IL2Rb, CD40, OX40 (CD134), CD80, CD86, CD27, ICOS, NKG2D, DAP10, DAP12, 2B4 (CD244), BTLA, CD30, GITR, CD226, CD79A, and HVEM.

In one embodiment, the co-stimulatory signaling domain is derived from 41BB. In one embodiment, the 41BB co-stimulatory signaling domain comprises the amino acid sequence set forth in SEQ ID NO: 8, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 8.

In one embodiment, the co-stimulatory signaling domain is derived from IL2Rb. In one embodiment, the IL2Rb co-stimulatory signaling domain comprises the amino acid sequence set forth in SEQ ID NO: 9, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 9.

In one embodiment, the co-stimulatory signaling domain is derived from CD40. In one embodiment, the CD40 co-stimulatory signaling domain comprises the amino acid sequence set forth in SEQ ID NO: 10, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 10.

In one embodiment, the co-stimulatory signaling domain is derived from OX40. In one embodiment, the OX40 co-stimulatory signaling domain comprises the amino acid sequence set forth in SEQ ID NO: 11, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 11.

In one embodiment, the co-stimulatory signaling domain is derived from CD80. In one embodiment, the CD80 co-stimulatory signaling domain comprises the amino acid sequence set forth in SEQ ID NO: 12, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 12.

In one embodiment, the co-stimulatory signaling domain is derived from CD86. In one embodiment, the CD86 co-stimulatory signaling domain comprises the amino acid sequence set forth in SEQ ID NO: 13, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 13.

In one embodiment, the co-stimulatory signaling domain is derived from CD27. In one embodiment, the CD27 co-stimulatory signaling domain comprises the amino acid sequence set forth in SEQ ID NO: 14, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 14.

In one embodiment, the co-stimulatory signaling domain is derived from ICOS. In one embodiment, the ICOS co-stimulatory signaling domain comprises the amino acid sequence set forth in SEQ ID NO: 15, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 15.

In one embodiment, the co-stimulatory signaling domain is derived from NKG2D. In one embodiment, the NKG2D co-stimulatory signaling domain comprises the amino acid sequence set forth in SEQ ID NO: 16, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 16.

In one embodiment, the co-stimulatory signaling domain is derived from DAP10. In one embodiment, the DAP10 co-stimulatory signaling domain comprises the amino acid sequence set forth in SEQ ID NO: 17, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 17.

In one embodiment, the co-stimulatory signaling domain is derived from DAP12. In one embodiment, the DAP12 co-stimulatory signaling domain comprises the amino acid sequence set forth in SEQ ID NO: 18, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 18.

In one embodiment, the co-stimulatory signaling domain is derived from 2B4 (CD244). In one embodiment, the 2B4 (CD244) co-stimulatory signaling domain comprises the amino acid sequence set forth in SEQ ID NO: 19, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 19.

In some embodiments, the CAR of the present disclosure comprises one costimulatory signaling domains. In some embodiments, the CAR of the present disclosure comprises two or more costimulatory signaling domains. In certain embodiments, the CAR of the present disclosure comprises two, three, four, five, six or more costimulatory signaling domains.

In some embodiments, the signaling domain(s) and costimulatory signaling domain(s) can be placed in any order. In some embodiments, the signaling domain is upstream of the costimulatory signaling domains. In some embodiments, the signaling domain is downstream from the costimulatory signaling domains. In the cases where two or more costimulatory domains are included, the order of the costimulatory signaling domains could be switched.

Non-limiting exemplary CAR regions and sequences are provided in Table 1.

TABLE 1

| | | | |
|---|---|---|---|
| | | UniProt | SEQ ID |
| CAR regions | Sequence | Id | NO |

CD19 CAR:

| | | | |
|---|---|---|---|
| GMCSFR | MLLLVTSLLLCELPHPAFLLIP | | 1 |
| Signal Peptide | | | |
| FMC63 VH | EVKLQESGPGLVAPSQSLSVTCTVSGVSLPDYGVSW | | 2 |
| | IRQPPRKGLEWLGVIWGSETTYYNSALKSRLTIIKDN | | |
| | SKSQVFLKMNSLQTDDTAIYYCAKHYYYGGSYAM | | |
| | DYWGQGTSVTVSS | | |
| Whitlow | GSTSGSGKPGSGEGSTKG | | 3 |
| Linker | | | |
| FMC63 VL | DIQMTQTTSSLSASLGDRVTISCRASQDISKYLNWY | | 4 |
| | QQKPDGTVKLLIYHTSRLHSGVPSRFSGSGSGTDYS | | |
| | LTISNLEQEDIATYFCQQGNTLPYTFGGGTKLEIT | | |
| CD28 | IEVMYPPPYLDNEKSNGTIIHVKGKHLCPSPLFPGPS | P10747-1 | 5 |
| (AA 114-220) | KPFWVLVVVGGVLACYSLLVTVAFIIFWVRSKRSR | | |
| | LLHSDYMNMTPRRPGPTRKHYQPYAPPRDFAAYRS | | |
| CD3-zeta | RVKFSRSADAPAYQQGQNQLYNELNLGRREEYDV | P20963-3 | 6 |
| isoform 3 | LDKRRGRDPEMGGKPRRKNPQEGLYNELQKDKMA | | |
| (AA 52-163) | EAYSEIGMKGERRRGKGHDGLYQGLSTATKDTYD | | |
| | ALHMQALPPR | | |
| FMC63 scFV | EVKLQESGPGLVAPSQSLSVTCTVSGVSLPDYGVS | | 7 |
| | WIRQPPRKGLEWLGVIWGSETTYYNSALKSRLTIIK | | |
| | DNSKSQVFLKMNSLQTDDTAIYYCAKHYYYGGSY | | |
| | AMDYWGQGTSVTVSSGSTSGSGKPGSGEGSTKGDI | | |
| | QMTQTTSSLSASLGDRVTISCRASQDISKYLNWYQQ | | |
| | KPDGTVKLLIYHTSRLHSGVPSRFSGSGSGTDYSLTI | | |
| | SNLEQEDIATYFCQQGNTLPYTFGGGTKLEIT | | |

Signaling Domains:

| | | | |
|---|---|---|---|
| 41BB | KRGRKKLLYIFKQPFMRPVQTTQEEDGCSCRFPEEE | Q07011 | 8 |
| (AA 214-255) | EGGCEL | | |
| IL2Rb | NCRNTGPWLKKVLKCNTPDPSKFFSQLSSEHGGDV | P14784 | 9 |
| (AA 266-551) | QKWLSSPFPSSSFSPGGLAPEISPLEVLERDKVTQLL | | |
| | PLNTDAYLSLQELQGQDPTHLV | | |
| CD40 | KKVAKKPTNKAPHPKQEPQEINFPDDLPGSNTAAPV | P25942 | 10 |
| (AA 216-277) | QETLHGCQPVTQEDGKESRISVQERQ | | |
| OX40 | ALYLLRRDQRLPPDAHKPPGGGSFRTPIQEEQADAH | P43489 | 11 |
| (AA 236-277) | STLAKI | | |
| CD80 | TYCFAPRCRERRRNERLRRESVRPV | P33681 | 12 |
| (AA 264-288) | | | |
| CD86 | KWKKKKRPRNSYKCGTNTMEREESEQTKKREKIHI | P42081 | 13 |
| (AA269-329) | PERSDEAQRVFKSSKTSSCDKSDTCF | | |
| CD27 | QRRKYRSNKGESPVEPAEPCHYSCPREEEGSTIPIQE | P26842 | 14 |
| (AA 213-260) | DYRKPEPACSP | | |
| ICOS | CWLTKKKYSSSVHDPNGEYMFMRAVNTAKKSRLT | Q9Y6W8 | 15 |
| (AA 162-199) | DVTL | | |
| NKG2D | MGWIRGRRSR HSWEMSEFHN YNLDLKKSDF | P26718 | 16 |
| (AA 1-51) | STRWQKQRCP VVKSKCRENAS | | |
| DAP10 | LCARPRRSPAQEDGKVYINMPGRG | Q9UBK5 | 17 |
| (AA 70-93) | | | |
| DAP12 | YFLGRLVPRGRGAAEAATRKQRITETESPYQELQGQ | O54885 | 18 |
| (AA 62-113) | RSDVYSDLNTQRPYYK | | |
| 2B4/CD244 | WRRKRKEKQSETSPKEFLTIYEDVKDLKTRRNHEQ | Q9BZW8 | 19 |
| (AA 251-370) | EQTFPGGGSTIYSMIQSQSSAPTSQEPAYTLYSLIQPS | | |
| | RKSGSRKRNHSPSFNSTIYEVIGKSQPKAQNPARLSR | | |
| | KELENFDVYS | | |
| CD3-zeta | RVKFSRSADAPAYQQGQNQLYNELNLGRREEYDV | P20963-3 | 6 |
| isoform 3 | LDKRRGRDPEMGGKPRRKNPQEGLYNELQKDKMA | | |
| (AA 52-163) | EAYSEIGMKGERRRGKGHDGLYQGLSTATKDTYD | | |
| | ALHMQALPPR | | |
| CD28 | RSKRSRLLHSDYMNMTPRRPGPTRKHYQPYAPPRD | P10747-1 | 20 |
| (AA 180-220) | FAAYRS | | |

Spacer/Hinge:

| | | | |
|---|---|---|---|
| CD8 | TTTPAPRPPTPAPTIASQPLSLRPEACRPAAGGAVHT | P01732 | 21 |
| (AA 136-182) | RGLDFACDIY | | |
| CD28 | IEVMYPPPYLDNEKSNGTIIHVKGKHLCPSPLFPGPS | P10747-1 | 22 |
| (AA 114-151) | KP | | |

Transmembrane:

| | | | |
|---|---|---|---|
| CD8 | IYIWAPLAGTCGVLLLSLVIT | P01732 | 23 |
| (AA 183-203) | | | |
| CD28 | FWVLVVVGGVLACYSLLVTVAFIIFWV | P10747-1 | 24 |
| (AA 153-179) | | | |

Linkers:

| | | |
|---|---|---|
| Whitlow | GSTSGSGKPGSGEGSTKG | 3 |
| Linker | | |
| (G₄S)₃ | GGGGSGGGGSGGGGS | 25 |
| Linker 3 | GGSEGKSSGSGSESKSTGGS | 26 |
| Linker 4 | GGGSGGGS | 27 |
| Linker 5 | GGGSGGGSGGGS | 28 |
| Linker 6 | GGGSGGGSGGGSGGGS | 29 |
| Linker 7 | GGGSGGGSGGGSGGGSGGGS | 30 |
| Linker 8 | GGGGSGGGGSGGGGSGGGGS | 31 |
| Linker 9 | GGGGSGGGGSGGGGSGGGGSGGGGS | 32 |
| Linker 10 | IRPRAIGGSKPRVA | 33 |
| Linker 11 | GKGGSGKGGSGKGGS | 34 |
| Linker 12 | GGKGSGGKGSGGKGS | 35 |
| Linker 13 | GGGKSGGGKSGGGKS | 36 |
| Linker 14 | GKGKSGKGKSGKGKS | 37 |
| Linker 15 | GGGKSGGKGSGKGGS | 38 |
| Linker 16 | GKPGSGKPGSGKPGS | 39 |
| Linker 17 | GKPGSGKPGSGKPGSGKPGS | 40 |
| Linker 18 | GKGKSGKGKSGKGKSGKGKS | 41 |
| Linker 19 | STAGDTHLGGEDFD | 42 |
| Linker 20 | GEGGSGEGGSGEGGS | 43 |
| Linker 21 | GGEGSGGEGSGGEGS | 44 |
| Linker 22 | GEGESGEGESGEGES | 45 |
| Linker 23 | GGGESGGEGSGEGGS | 46 |
| Linker 24 | GEGESGEGESGEGESGEGES | 47 |
| Linker 25 | GSTSGSGKPGSGEGSTKG | 48 |
| Linker 26 | PRGASKSGSASQTGSAPGS | 49 |
| Linker 27 | GTAAAGAGAAGGAAAGAAG | 50 |
| Linker 28 | GTSGSSGSGSGGSGSGGGG | 51 |
| Linker 29 | GKPGSGKPGSGKPGSGKPGS | 52 |
| Linker 30 | GSGS | 53 |
| Linker 31 | APAPAPAPAP | 54 |
| Linker 32 | APAPAPAPAPAPAPAPAPAP | 55 |
| Linker 33 | AEAAAKEAAAKEAAAAKEAAAAKEAAAAKAAA | 56 |

In some embodiments, the antigen-binding domain of the second polypeptide binds to an antigen. The antigen-binding domain of the second polypeptide can bind to more than one antigen or more than one epitope in an antigen. For example, the antigen-binding domain of the second polypeptide can bind to two, three, four, five, six, seven, eight or more antigens. As another example, the antigen-binding domain of the second polypeptide can bind to two, three, four, five, six, seven, eight or more epitopes in the same antigen.

The choice of antigen-binding domain may depend upon the type and number of antigens that define the surface of a target cell. For example, the antigen-binding domain can be chosen to recognize an antigen that acts as a cell surface marker on target cells associated with a particular disease state. In certain embodiments, the CARs of the present disclosure can be genetically modified to target a tumor antigen of interest by way of engineering a desired antigen-binding domain that specifically binds to an antigen (e.g., on a tumor cell). Non-limiting examples of cell surface markers that can act as targets for the antigen-binding domain in the CAR of the disclosure include those associated with tumor cells or autoimmune diseases.

In some embodiments, the antigen-binding domain binds to at least one tumor antigen or autoimmune antigen.

In some embodiments, the antigen-binding domain binds to at least one tumor antigen. In some embodiments, the antigen-binding domain binds to two or more tumor antigens. In some embodiments, the two or more tumor antigens are associated with the same tumor. In some embodiments, the two or more tumor antigens are associated with different tumors.

In some embodiments, the antigen-binding domain binds to at least one autoimmune antigen. In some embodiments, the antigen-binding domain binds to two or more autoimmune antigens. In some embodiments, the two or more autoimmune antigens are associated with the same autoimmune disease. In some embodiments, the two or more autoimmune antigens are associated with different autoimmune diseases.

In some embodiments, the tumor antigen is associated with glioblastoma, ovarian cancer, cervical cancer, head and neck cancer, liver cancer, prostate cancer, pancreatic cancer, renal cell carcinoma, bladder cancer, or hematologic malignancy. Non-limiting examples of tumor antigen associated with glioblastoma include HER2, EGFRvIII, EGFR, CD133, PDGFRA, FGFR1, FGFR3, MET, CD70, ROBOland IL13Ra2. Non-limiting examples of tumor antigens associated with ovarian cancer include FOLR1, FSHR, MUC16, MUC1, Mesothelin, CA125, EpCAM, EGFR, PDGFRa, Nectin-4, and B7H4. Non-limiting examples of the tumor antigens associated with cervical cancer or head and neck cancer include GD2, MUC1, Mesothelin, HER2, and EGFR. Non-limiting examples of tumor antigen associated with liver cancer include Claudin 18.2, GPC-3, EpCAM, cMET, and AFP. Non-limiting examples of tumor antigens associated with hematological malignancies include CD22, CD79 (CD79a and/or CD79b), BCMA, GPRC5D, SLAM F7, CD33, CLL1, CD123, and CD70. Non-limiting examples of tumor antigens associated with bladder cancer include Nectin-4 and SLITRK6.

Additional examples of antigens that can be targeted by the antigen-binding domain include, but are not limited to, alpha-fetoprotein, A3, antigen specific for A33 antibody, Ba 733, BrE3-antigen, carbonic anhydrase EX, CD1, CD1a, CD3, CD5, CD15, CD16, CD19, CD20, CD21, CD22, CD23, CD25, CD30, CD33, CD38, CD45, CD74, CD79a, CD80, CD123, CD138, colon-specific antigen-p (CSAp), CEA (CEACAM5), CEACAM6, CSAp, EGFR, EGP-I, EGP-2, Ep-CAM, EphA1, EphA2, EphA3, EphA4, EphA5, EphA6, EphA7, EphA8, EphA10, EphB1, EphB2, EphB3, EphB4, EphB6, FIt-I, Flt-3, folate receptor, HLA-DR, human chorionic gonadotropin (HCG) and its subunits, hypoxia inducible factor (HIF-I), Ia, IL-2, IL-6, IL-8, insulin growth factor-1 (IGF-I), KC4-antigen, KS-1-antigen, KS1-4, Le-Y, macrophage inhibition factor (MIF), MAGE, MUC2, MUC3, MUC4, NCA66, NCA95, NCA90, antigen specific for PAM-4 antibody, placental growth factor, p53, prostatic acid phosphatase, PSA, PSMA, RS5, 5100, TAC, TAG-72, tenascin, TRAIL receptors, Tn antigen, Thomson-Friedenreich antigens, tumor necrosis antigens, VEGF, ED-B fibronectin, 17-1A-antigen, an angiogenesis marker, an oncogene marker or an oncogene product.

In one embodiment, the antigen targeted by the antigen-binding domain is CD19. In one embodiment, the antigen-binding domain comprises an anti-CD19 scFv. In one embodiment, the anti-CD19 scFv comprises a heavy chain variable region (VH) comprising the amino acid sequence set forth in SEQ ID NO: 2, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 2. In one embodiment, the anti-CD19 scFv comprises a light chain variable region (VL) comprising the amino acid sequence set forth in SEQ ID NO: 4, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 4. In one embodiment, the anti-CD19 scFv comprises the amino acid sequence set forth in SEQ ID NO: 7, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 7.

In some embodiments, the antigen is associated with an autoimmune disease or disorder. Such antigens can be derived from cell receptors and cells which produce “self”-directed antibodies. In some embodiments, the antigen is associated with an autoimmune disease or disorder such as Rheumatoid arthritis (RA), multiple sclerosis (MS), Sjögren's syndrome, Systemic lupus erythematosus, sarcoidosis, Type 1 diabetes mellitus, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Myasthenia gravis, Hashimoto's thyroiditis, Graves' disease, chronic inflammatory demyelinating polyneuropathy, Guillain-Barre syndrome, Crohn's disease or ulcerative colitis.

In some embodiments, autoimmune antigens that can be targeted by the CAR disclosed herein include but are not limited to platelet antigens, myelin protein antigen, Sm antigens in snRNPs, islet cell antigen, Rheumatoid factor, and anticitrullinated protein. citrullinated proteins and peptides such as CCP-1, CCP-2 (cyclical citrullinated peptides), fibrinogen, fibrin, vimentin, fillaggrin, collagen I and II peptides, alpha-enolase, translation initiation factor 4G1, perinuclear factor, keratin, Sa (cytoskeletal protein vimentin), components of articular cartilage such as collagen II, IX, and XI, circulating serum proteins such as RFs (IgG, IgM), fibrinogen, plasminogen, ferritin, nuclear components such as RA33/hnRNP A2, Sm, eukaryotic translation elongation factor 1 alpha 1, stress proteins such as HSP-65, -70, -90, BiP, inflammatory/immune factors such as B7-H1, IL-1 alpha, and IL-8, enzymes such as calpastatin, alpha-enolase, aldolase-A, dipeptidyl peptidase, osteopontin, glucose-6-phosphate isomerase, receptors such as lipocortin 1, neutrophil nuclear proteins such as lactoferrin and 25-35 kD nuclear protein, granular proteins such as bactericidal permeability increasing protein (BPI), elastase, cathepsin G, myeloperoxidase, proteinase 3, platelet antigens, myelin protein antigen, islet cell antigen, rheumatoid factor, histones, ribosomal P proteins, cardiolipin, vimentin, nucleic acids such as dsDNA, ssDNA, and RNA, ribonuclear particles and proteins such as Sm antigens (including but not limited to SmD's and SmB7B), U1RNP, A2/B1 hnRNP, Ro (SSA), and La (SSB) antigens.

In various embodiments, the scFv fragment used in the CAR of the present disclosure can include a linker between the VH and VL domains. The linker can be a peptide linker and can include any naturally occurring amino acid. Exemplary amino acids that can be included into the linker are Gly, Ser Pro, Thr, Glu, Lys, Arg, Ile, Leu, His and The. The linker should have a length that is adequate to link the VH and the VL in such a way that they form the correct conformation relative to one another so that they retain the desired activity, such as binding to an antigen. The linker can be about 5-50 amino acids long. In some embodiments, the linker is about 10-40 amino acids long. In some embodiments, the linker is about 10-35 amino acids long. In some embodiments, the linker is about 10-30 amino acids long. In some embodiments, the linker is about 10-25 amino acids long. In some embodiments, the linker is about 10-20 amino acids long. In some embodiments, the linker is about 15-20 amino acids long. Exemplary linkers that can be used are Gly rich linkers, Gly and Ser containing linkers, Gly and Ala containing linkers, Ala and Ser containing linkers, and other flexible linkers.

In one embodiment, the linker is a Whitlow linker. In one embodiment, the Whitlow linker comprises the amino acid sequence set forth in SEQ ID NO: 3, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 3. In another embodiment, the linker is a (G₄S)₃ inker. In one embodiment, the (G₄S)₃ inker comprises the amino acid sequence set forth in SEQ ID NO: 25, or a variant thereof having at least 50, at least 55, at least 60, at least 65, at least 70, at least 75, at least 80, at least 85, at least 90, at least 95, at least 96, at least 97, at least 98 or at least 99%, sequence identity with SEQ ID NO: 25.

Other linker sequences can include portions of immunoglobulin hinge area, CL or CH1 derived from any immunoglobulin heavy or light chain isotype. Exemplary linkers that can be used include any of SEQ ID NOs: 26-56 in Table 1. Additional linkers are described for example in Int. Pat. Publ. No. WO2019/060695, incorporated by reference herein in its entirety.

### III. Artificial Cell Death/Reporter System Polypeptide

In a general aspect, the application provides a polynucleotide encoding an artificial cell death polypeptide and an immune effector cell engineered to express such a polypeptide.

As used herein, the term “an artificial cell death polypeptide” refers to an engineered protein designed to prevent potential toxicity or otherwise adverse effects of a cell therapy. The artificial cell death polypeptide could mediate induction of apoptosis, inhibition of protein synthesis, DNA replication, growth arrest, transcriptional and post-transcriptional genetic regulation and/or antibody-mediated depletion. In some instance, the artificial cell death polypeptide is activated by an exogenous molecule, e.g., an antibody, anti-viral drug, or radioisotopic conjugate drugs, that when activated, triggers apoptosis and/or cell death of a therapeutic cell.

In certain embodiments, the artificial cell death polypeptide comprises a viral enzyme that is recognized by an antiviral drug. In certain embodiments, the viral enzyme is a herpes simplex virus thymidine kinase (HSV-tk). In certain embodiments, the HSV-tk comprises or consists of an amino acid sequence at least 90%, such as at least 90%, 91%, 82%, 93%, 94%, 95%, 96%, 97%, 98%, 99% or 100%, identical to SEQ ID NO: 71, preferably the amino acid sequence of SEQ ID NO: 71. This enzyme phosphorylates the nontoxic prodrugs acyclovir or ganciclovir, which then become phosphorylated by endogenous kinases to GCV-triphosphate, causing chain termination and single-strand breaks upon incorporation into DNA, thereby killing dividing cells. In certain embodiments, expression of the viral enzyme in an engineered immune cell expressing a chimeric antigen receptor (CAR) induces cell death of the engineered immune cell when the cell is contacted with one or more antiviral drugs. In certain embodiments, the one or more antiviral drugs comprise acyclovir or a derivative thereof, or ganciclovir or a derivative thereof.

In another general aspect, the application provides a polynucleotide encoding a reporter system and an immune effector cell engineered to express such a reporter system. As used herein, the term “reporter system” refers to an engineered protein that, in combination with an imaging probe, can be used to mark cells.

The reporter system polypeptide comprises an antigen targeted by an entity, such as a small molecule compound, a radioisotopic conjugate, or an antibody or an antigen binding fragment thereof. In certain embodiments, the antigen is a prostate-specific membrane antigen (PSMA) polypeptide, also referred to as Glutamate carboxypeptidase 2. PSMA is a type II membrane protein that is targeted to the secretary pathway by its transmembrane domain, which biochemically resembles a signal sequence without being cleaved. In certain embodiments, the reporter system polypeptide comprises a prostate-specific membrane antigen (PSMA) extracellular domain or fragment thereof.

In certain embodiments, the PSMA polypeptide is a truncated variant as described in Intl. Pat. Applications WO2015143029A1 and WO2018187791A1, the disclosures of which are incorporated by reference into the present application in entirety. In certain embodiments, the prostate-specific membrane antigen (PSMA) polypeptide comprises or consists of an amino acid sequence at least 90%, such as at least 90%, 91%, 82%, 93%, 94%, 95%, 96%, 97%, 98%, 99% or 100%, identical to SEQ ID NO: 72, preferably the amino acid sequence of SEQ ID NO: 72. In certain embodiments, the PSMA antigen may also function as an artificial cell death polypeptide since expression of truncated PSMA in an engineered immune cell expressing a chimeric antigen receptor (CAR) induces cell death of the engineered immune cell when the cell is contacted with a radioisotopic conjugate drug that binds to PSMA via a peptide. PSMA-targeting compounds are described in WO2010/108125, the disclosure of which is incorporated herein by reference.

In another general aspect, the application provides a combined artificial cell death/reporter system polypeptide that can function as an artificial cell death polypeptide, a reporter system, or both an artificial cell death polypeptide and a reporter system.

In certain embodiments, the polynucleotide encodes a combined artificial cell death/reporter system polypeptide. A combined artificial cell death/reporter system polypeptide can function as an artificial cell death polypeptide, a reporter system, or both an artificial cell death polypeptide and a reporter system. Having the combined artificial cell death and reporter system in a single polynucleotide that can be expressed as a single polypeptide has the advantage of reducing the number of gene edits of the immune effector cell.

In certain embodiments, the artificial cell death/reporter system polypeptide comprises an intracellular domain having a herpes simplex virus thymidine kinase (HSV-tk) and a linker, a transmembrane region, and an extracellular domain comprising a prostate-specific membrane antigen (PSMA) extracellular domain or fragment thereof.

In certain embodiments, the linker comprises or consists of an amino acid sequence selected from the group consisting of SEQ ID NO: 25 to 56, such as the linker consisting of the amino acid sequence of SEQ ID NO: 48.

In certain embodiments, the linker comprises an autoprotease peptide sequence, such as an autoprotease peptide sequence selected from the group consisting of porcine teschovirus-1 2A

(P2A), thosea asigna virus 2A (T2A), equine rhinitis A virus 2A (E2A), foot-and-mouth disease virus 18 2A (F2A). In a particular embodiment, the autoprotease peptide is a thosea asigna virus 2A (T2A) peptide comprising the amino acid of SEQ ID NO: 75.

In certain embodiments, the artificial cell death/reporter system polypeptide comprises the HSV-tk fused to a truncated variant PSMA polypeptide via the linker.

In certain embodiments the artificial cell death/reporter system polypeptide comprises an amino acid sequence having at least 90%, 91%, 92%, 93%, 94%, 95%, 96%, 97%, 98%, 99% or 100% sequence identity to SEQ ID NO: 73.

In certain embodiments the artificial cell death/reporter system polypeptide comprises an amino acid sequence having at least 90%, 91%, 92%, 93%, 94%, 95%, 96%, 97%, 98%, 99% or 100% sequence identity to SEQ ID NO: 76.

Also provided is an embodiment in which the artificial cell death polypeptide/reporter system is combined with an immune checkpoint inhibitor, cluster of differentiation 24 (CD24), to provide the cell with a “don't eat me signal” to escape macrophage-mediated phagocytosis through expression of anti-phagocytic signals. Emerging data indicate a role for innate immune checkpoints in immune evasion, whereby tumors can escape macrophage-mediated phagocytosis through expression of anti-phagocytic signals. One such ‘don't eat me’ signal, CD24, orchestrates a novel innate immune checkpoint through interaction with the inhibitory receptor sialic acid-binding Ig-like lectin 10 (Siglec-10) on tumor-associated macrophages (TAMs) (Barkal et al., Nature, 2019 August; 572(7769) 392-396). In certain embodiments, the combined artificial cell death/reporter system/CD24 polypeptide comprises a cluster of differentiation 24 (CD24) fused to a prostate-specific membrane antigen (PSMA) extracellular domain or fragment thereof via a peptide linker. The CD24 is then co-expressed with the PSMA polypeptide on the cell surface.

In certain embodiments, the CD24 comprises or consists of an amino acid sequence at least 90%, such as at least 90%, 91%, 82%, 93%, 94%, 95%, 96%, 97%, 98%, 99% or 100%, identical to SEQ ID NO: 78.

In certain embodiments, the artificial cell death/reporter system/CD24 polypeptide comprises the CD24 fused to a truncated variant PSMA polypeptide via the linker.

In certain embodiments, the artificial cell death/reporter system/CD24 polypeptide comprises an amino acid sequence having at least 90%, 91%, 92%, 93%, 94%, 95%, 96%, 97%, 98%, 99% or 100% sequence identity to SEQ ID NO: 79.

### IV. HLA Expression

In one aspect, WIC I and/or WIC II knock-out and/or knock down can be incorporated in the cells for use in “allogeneic” cell therapies, in which cells are harvested from a subject, modified to knock-out or knock-down, e.g., disrupt, B2M, TAP 1, TAP 2, Tapasin, RFXANK, CIITA, RFX5 and RFXAP gene expression, and then returned to a different subject. Knocking out or knocking down the B2M, TAP 1, TAP 2, Tapasin, RFXANK, CIITA, RFX5 and RFXAP genes as described herein can: (1) prevent GvH response; (2) prevent HvG response; and/or (3) improve T cell safety and efficacy. Accordingly, in certain embodiments, a presently disclosed invention comprises independently knocking out and/or knocking down one or more genes selected from the group consisting of B2M, TAP 1, TAP 2, Tapasin, RFXANK, CIITA, RFX5 and RFXAP genes in a T cell. In certain embodiments, a presently disclosed method comprises independently knocking out and/or knocking down two genes selected from the group consisting B2M, TAP 1, TAP 2, Tapasin, RFXANK, CIITA, RFX5 and RFXAP genes in a T cell, in particular, B2M and CIITA to achieve class I and II HLA disruption. In certain embodiments, an iPSC or derivative cell thereof of the application can be further modified by introducing an exogenous polynucleotide encoding one or more proteins related to immune evasion, such as non-classical HLA class I proteins (e.g., HLA-E and HLA-G). In particular, disruption of the B2M gene eliminates surface expression of all MHC class I molecules, leaving cells vulnerable to lysis by NK cells through the “missing self” response. Exogenous HLA-E expression can lead to resistance to NK-mediated lysis (Gornalusse et al., Nat Biotechnol. 2017; 35(8): 765-772).

In certain embodiments, the iPSC or derivative cell thereof comprises an exogenous polypeptide encoding at least one of a human leukocyte antigen E (HLA-E) and human leukocyte antigen G (HLA-G). In a particular embodiment, the HLA-E comprises an amino acid sequence at least 90%, such as at least 90%, 91%, 82%, 93%, 94%, 95%, 96%, 97%, 98%, 99% or 100%, identical to SEQ ID NO: 65, preferably the amino acid sequence of SEQ ID NO: 65. In a particular embodiment, the HLA-G comprises an amino acid sequence at least 90%, such as at least 90%, 91%, 92%, 93%, 94%, 95%, 96%, 97%, 98%, 99% or 100%, identical to SEQ ID NO: 68, preferably SEQ ID NO: 68.

In certain embodiments, the exogenous polynucleotide encodes a polypeptide comprising a signal peptide operably linked to a mature B2M protein that is fused to an HLA-E via a linker. In a particular embodiment, the exogenous polypeptide comprises an amino acid sequence at least sequence at least 90%, such as at least 90%, 91%, 82%, 93%, 94%, 95%, 96%, 97%, 98%, 99% or 100%, identical to SEQ ID NO: 66.

In other embodiments, the exogenous polynucleotide encodes a polypeptide comprising a signal peptide operably linked to a mature B2M protein that is fused to an HLA-G via a linker.

In a particular embodiment, the exogenous polypeptide comprises an amino acid sequence at least sequence at least 90%, such as at least 90%, 91%, 82%, 93%, 94%, 95%, 96%, 97%, 98%, 99% or 100%, identical to SEQ ID NO: 69.

### V. Other Optional Genome Edits

In certain embodiments, a cell of the application further comprises an exogenous polynucleotide encoding interleukin 15 (IL-15) and/or interleukin (IL-15) receptor or a variant or truncation thereof. As used herein “Interleukin-15” or “IL-15” refers to a cytokine that regulates T and NK cell activation and proliferation. A “functional portion” (“biologically active portion”) of IL-15 refers to a portion of IL-15 that retains one or more functions of full length or mature IL-15. Such functions include the promotion of NK cell survival, regulation of NK cell and T cell activation and proliferation as well as the support of NK cell development from hematopoietic stem cells. As will be appreciated by those of skill in the art, the sequence of a variety of IL-15 molecules are known in the art. In certain embodiments, the IL-15 is a wild-type IL-15. In certain embodiments, the IL-15 is a human IL-15.

In another embodiment, the cell of the application further comprises an exogenous polynucleotide encoding a non-naturally occurring variant of FcγRIII (CD16), for example, hnCD16 (see, e.g., Zhu et al., Blood 2017, 130:4452, the contents of which are incorporated herein in their entirety by reference). As used herein, the term “hnCD16a” refers to a high affinity, non-cleavable variant of CD16 (a low-affinity Fcγ receptor involved in antibody-dependent cellular cytotoxicity (ADCC). Typically, CD16 is cleaved during ADCC by proteases whereas the hnCD16 CAR does not undergo this cleavage and thus sustains an ADCC signal longer. In some embodiments, the hnCD 16 is as disclosed in Blood 2016 128:3363, the entire contents of which is expressly incorporated herein by reference.

In another embodiment, a cell of the application further comprises an exogenous polynucleotide encoding interleukin 12 (IL-12) or interleukin 21 (IL-21) or a variant thereof.

In another embodiment, a cell of the application further comprises an exogenous polynucleotide encoding leukocyte surface antigen cluster of differentiation CD47 (CD47) as an NK inhibitory modality to overcome host-versus-graft immunoreactivity for allogeneic applications. As used herein, the term “CD47,” also sometimes referred to as “integrin associated protein” (IAP), refers to a transmembrane protein that in humans is encoded by the CD47gene. CD47 belongs to the immunoglobulin superfamily, partners with membrane integrins, and also binds the ligands thrombospondin-1 (TSP-1) and signal-regulatory protein alpha (SIRPa). CD47 acts as a signal to macrophages that allows CD47-expressing cells to escape macrophage attack. See, e.g., Deuse-T, et al., Nature Biotechnology 2019 37: 252-258, the entire contents of which are incorporated herein by reference.

In another embodiment, a cell of the application further comprises an exogeneous polynucleotide encoding a constitutively active IL-7 receptor or variant thereof. IL-7 has a critical role in the development and maturation of T cells. It promotes the generation of naïve and central memory T cell subsets and regulates their homeostasis. It has previously been reported that IL-7 prolonged the survival time of tumor-specific T cells in vivo. Cancer Medicine. 2014; 3(3):550-554. In previous studies, it has been reported that a constitutively activated IL-7 receptor (C7R) could result in IL-7 signaling in the absence of a ligand or with the existence of gamma chain (γc) of a coreceptor. Shum et al, Cancer Discovery. 2017; 7(11):1238-1247. Insertion of a transmembrane domain such as cysteine and/or proline resulted in the homodimerization of IL-7Rα. Upon the formation of a homodimer, cross-phosphorylation of JAK1/JAK1 activates STATS, thereby activating the downstream signaling of IL-7. Constructs for such constitutively activated IL-7 receptor (C7R) compositions are disclosed in WO2018/038945, the contents of which are hereby incorporated by reference into the present application.

In another embodiment, a cell of the application further comprises an exogenous polynucleotide encoding one or more imaging or reporter proteins, such as PSMA. For example, the cell can contain an exogeneous polynucleotide encoding prostate-specific membrane antigen (PSMA) as an imaging reporter in accordance with the disclosures of WO2015/143029 and WO2018/187791, the disclosures of which are incorporated herein by reference.

In one embodiment of the above described cell, the genomic editing at one or more selected sites can comprise insertions of one or more exogenous polynucleotides encoding other additional artificial cell death polypeptides proteins, targeting modalities, receptors, signaling molecules, transcription factors, pharmaceutically active proteins and peptides, drug target candidates, or proteins promoting engraftment, trafficking, homing, viability, self-renewal, persistence, and/or survival of the genome-engineered iPSCs or derivative cells thereof.

In some embodiments, the exogenous polynucleotides for insertion are operatively linked to (1) one or more exogenous promoters comprising CMV, EFla, PGK, CAG, UBC, or other constitutive, inducible, temporal-, tissue-, or cell type-specific promoters; or (2) one or more endogenous promoters comprised in the selected sites comprising AAVS1, CCR5, ROSA26, collagen, HTRP, Hll, beta-2 microglobulin, GAPDH, TCR or RUNX1, or other locus meeting the criteria of a genome safe harbor. In some embodiments, the genome-engineered iPSCs generated using the above method comprise one or more different exogenous polynucleotides encoding proteins comprising caspase, thymidine kinase, cytosine deaminase, B-cell CD20, ErbB2 or CD79b wherein when the genome-engineered iPSCs comprise two or more suicide genes, the suicide genes are integrated in different safe harbor locus comprising AAVS1, CCR5, ROSA26, collagen, HTRP, Hll, Hll, beta-2 microglobulin, GAPDH, TCR or RUNX1. Other exogenous polynucleotides encoding proteins can include those encoding PET reporters, homeostatic cytokines, and inhibitory checkpoint inhibitory proteins such as PD1, PD-L1, and CTLA4 as well as proteins that target the CD47/signal regulatory protein alpha (SIRPa) axis. In some other embodiments, the genome-engineered iPSCs generated using the method provided herein comprise in/del at one or more endogenous genes associated with targeting modality, receptors, signaling molecules, transcription factors, drug target candidates, immune response regulation and modulation, or proteins suppressing engraftment, trafficking, homing, viability, self-renewal, persistence, and/or survival of the iPSCs or derivative cells thereof.

In addition, the modified γδ cells can exhibit one or more edits in their genome that results in a loss-of-function in a target gene. A loss-of-function of a target gene is characterized by a decrease in the expression of a target gene based on a genomic modification, e.g., an RNA-guided nuclease-mediated cut in the target gene that results in an inactivation, or in diminished expression or function, of the encoded gene product. Examples of genes that can be targeted for loss of function include B2M, PD-1, CISH, CIITA, HLA class II histocompatibility alpha chain genes (e.g. HLA-DQA1, HLA-DRA, HLA-DPA1, HLA-DMA-HLA-DQA2 and or HLA-DOA), HLA Class II histocompatabilty beta chain genes (e.g. HLA-DMB, HLA-DOB, HLA-DPB1, HLA-DQB1, HLA-DQB2, HLA-DQB3, HLA-DRB1, HLADRB3, HLA-DRB4, and/or HLA-DRB5), CD32B, CTLA4, NKG2A, BIM, CCR5, CCR7, CD96, CDK8, CXCR3, EP4 (PGE2 RECEPTOR), Fas, GITR, IL1R8, KIRDL1, KIR2DL1-3, LAG3, SOCS genes, Sortilin, TRAC, RAG1, RAG2 and NLRC5.

The modified cells of the application can exhibit any of the edits described, as well as any combination of such edits described.

VI. Targeted Genome Editing at Selected Locus in iPSCs

According to embodiments of the application, one or more of the exogenous polynucleotides are integrated at one or more loci on the chromosome of an iPSC.

Genome editing, or genomic editing, or genetic editing, as used interchangeably herein, is a type of genetic engineering in which DNA is inserted, deleted, and/or replaced in the genome of a targeted cell. Targeted genome editing (interchangeable with “targeted genomic editing” or “targeted genetic editing”) enables insertion, deletion, and/or substitution at pre-selected sites in the genome. When an endogenous sequence is deleted or disrupted at the insertion site during targeted editing, an endogenous gene comprising the affected sequence can be knocked-out or knocked-down due to the sequence deletion or disruption. Therefore, targeted editing can also be used to disrupt endogenous gene expression with precision. Similarly used herein is the term “targeted integration,” referring to a process involving insertion of one or more exogenous sequences at pre-selected sites in the genome, with or without deletion of an endogenous sequence at the insertion site.

Targeted editing can be achieved either through a nuclease-independent approach, or through a nuclease-dependent approach. In the nuclease-independent targeted editing approach, homologous recombination is guided by homologous sequences flanking an exogenous polynucleotide to be inserted, through the enzymatic machinery of the host cell.

Alternatively, targeted editing could be achieved with higher frequency through specific introduction of double strand breaks (DSBs) by specific rare-cutting endonucleases. Such nuclease-dependent targeted editing utilizes DNA repair mechanisms including non-homologous end joining (NHEJ), which occurs in response to DSBs. Without a donor vector containing exogenous genetic material, the NHEJ often leads to random insertions or deletions (in/dels) of a small number of endogenous nucleotides. In comparison, when a donor vector containing exogenous genetic material flanked by a pair of homology arms is present, the exogenous genetic material can be introduced into the genome during homology directed repair (HDR) by homologous recombination, resulting in a “targeted integration.”

Available endonucleases capable of introducing specific and targeted DSBs include, but not limited to, zinc-finger nucleases (ZFN), transcription activator-like effector nucleases (TALEN) and RNA-guided CRISPR (Clustered Regular Interspaced Short Palindromic Repeats) systems. Additionally, DICE (dual integrase cassette exchange) system utilizing phiC31 and Bxbl integrases is also a promising tool for targeted integration.

ZFNs are targeted nucleases comprising a nuclease fused to a zinc finger DNA binding domain. By a “zinc finger DNA binding domain” or “ZFBD” it is meant a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include, but not limited to, C2H2 zinc fingers, C3H zinc fingers, and C4 zinc fingers. A “designed” zinc finger domain is a domain not occurring in nature whose design/composition results principally from rational criteria, e.g., application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A “selected” zinc finger domain is a domain not found in nature whose production results primarily from an empirical process such as phage display, interaction trap or hybrid selection. ZFNs are described in greater detail in U.S. Pat. Nos. 7,888,121 and 7,972,854, the complete disclosures of which are incorporated herein by reference. The most recognized example of a ZFN in the art is a fusion of the Fokl nuclease with a zinc finger DNA binding domain.

A TALEN is a targeted nuclease comprising a nuclease fused to a TAL effector DNA binding domain. By “transcription activator-like effector DNA binding domain”, “TAL effector DNA binding domain”, or “TALE DNA binding domain” it is meant the polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in U.S. Patent Application No. 2011/0145940, which is herein incorporated by reference. The most recognized example of a TALEN in the art is a fusion polypeptide of the Fokl nuclease to a TAL effector DNA binding domain.

Additional examples of targeted nucleases suitable for the present application include, but not limited to Spol 1, Bxbl, phiC3 1, R4, PhiBTl, and Wp/SPBc/TP901-1, whether used individually or in combination.

Other non-limiting examples of targeted nucleases include naturally occurring and recombinant nucleases; CRISPR related nucleases from families including cas, cpf, cse, csy, csn, csd, cst, csh, csa, csm, and cmr; restriction endonucleases; meganucleases; homing endonucleases, and the like. As an example, CRISPR/Cas9 requires two major components: (1) a Cas9 endonuclease and (2) the crRNA-tracrRNA complex. When co-expressed, the two components form a complex that is recruited to a target DNA sequence comprising PAM and a seeding region near PAM. The crRNA and tracrRNA can be combined to form a chimeric guide RNA (gRNA) to guide Cas9 to target selected sequences. These two components can then be delivered to mammalian cells via transfection or transduction. As another example, CRISPR/Cpfl comprises two major components: (1) a CPfl endonuclease and (2) a crRNA. When co-expressed, the two components form a ribobnucleoprotein (RNP) complex that is recruited to a target DNA sequence comprising PAM and a seeding region near PAM. The crRNA can be combined to form a chimeric guide RNA (gRNA) to guide Cpfl to target selected sequences. These two components can then be delivered to mammalian cells via transfection or transduction.

MAD7 is an engineered Cas12a variant originating from the bacterium Eubacterium rectale that has a preference for 5′-TTTN-3′ and 5′-CTTN-3′ PAM sites and does not require a tracrRNA. See, for example, PCT Publication No. 2018/236548, the disclosure of which is incorporated herein by reference.

DICE mediated insertion uses a pair of recombinases, for example, phiC31 and Bxbl, to provide unidirectional integration of an exogenous DNA that is tightly restricted to each enzymes' own small attB and attP recognition sites. Because these target att sites are not naturally present in mammalian genomes, they must be first introduced into the genome, at the desired integration site. See, for example, U.S. Application Publication No. 2015/0140665, the disclosure of which is incorporated herein by reference.

One aspect of the present application provides a construct comprising one or more exogenous polynucleotides for targeted genome integration. In one embodiment, the construct further comprises a pair of homologous arm specific to a desired integration site, and the method of targeted integration comprises introducing the construct to cells to enable site specific homologous recombination by the cell host enzymatic machinery. In another embodiment, the method of targeted integration in a cell comprises introducing a construct comprising one or more exogenous polynucleotides to the cell, and introducing a ZFN expression cassette comprising a DNA-binding domain specific to a desired integration site to the cell to enable a ZFN-mediated insertion. In yet another embodiment, the method of targeted integration in a cell comprises introducing a construct comprising one or more exogenous polynucleotides to the cell, and introducing a TALEN expression cassette comprising a DNA-binding domain specific to a desired integration site to the cell to enable a TALEN-mediated insertion. In another embodiment, the method of targeted integration in a cell comprises introducing a construct comprising one or more exogenous polynucleotides to the cell, introducing a Cpfl expression cassette, and a gRNA comprising a guide sequence specific to a desired integration site to the cell to enable a Cpfl-mediated insertion. In another embodiment, the method of targeted integration in a cell comprises introducing a construct comprising one or more exogenous polynucleotides to the cell, introducing a Cas9 expression cassette, and a gRNA comprising a guide sequence specific to a desired integration site to the cell to enable a Cas9-mediated insertion. In still another embodiment, the method of targeted integration in a cell comprises introducing a construct comprising one or more att sites of a pair of DICE recombinases to a desired integration site in the cell, introducing a construct comprising one or more exogenous polynucleotides to the cell, and introducing an expression cassette for DICE recombinases, to enable DICE-mediated targeted integration.

Sites for targeted integration include, but are not limited to, genomic safe harbors, which are intragenic or extragenic regions of the human genome that, theoretically, are able to accommodate predictable expression of newly integrated DNA without adverse effects on the host cell or organism. In certain embodiments, the genome safe harbor for the targeted integration is one or more loci of genes selected from the group consisting of AAVS1, CCR5, ROSA26, collagen, HTRP, Hll, GAPDH, TCR and RUNX1 genes.

In other embodiments, the site for targeted integration is selected for deletion or reduced expression of an endogenous gene at the insertion site. As used herein, the term “deletion” with respect to expression of a gene refers to any genetic modification that abolishes the expression of the gene. Examples of “deletion” of expression of a gene include, e.g., a removal or deletion of a DNA sequence of the gene, an insertion of an exogenous polynucleotide sequence at a locus of the gene, and one or more substitutions within the gene, which abolishes the expression of the gene.

Genes for target deletion include, but are not limited to, genes of major histocompatibility complex (MHC) class I and MHC class II proteins. Multiple MHC class I and class II proteins must be matched for histocompatibility in allogeneic recipients to avoid allogeneic rejection problems. “MHC deficient”, including MHC-class I deficient, or MHC-class II deficient, or both, refers to cells that either lack, or no longer maintain, or have reduced level of surface expression of a complete MHC complex comprising a MHC class I protein heterodimer and/or a MHC class II heterodimer, such that the diminished or reduced level is less than the level naturally detectable by other cells or by synthetic methods. MHC class I deficiency can be achieved by functional deletion of any region of the MHC class I locus (chromosome 6p21), or deletion or reducing the expression level of one or more MHC class-I associated genes including, not being limited to, beta-2 microglobulin (B2M) gene, TAP 1 gene, TAP 2 gene and Tapasin genes. For example, the B2M gene encodes a common subunit essential for cell surface expression of all MHC class I heterodimers. B2M null cells are MHC-I deficient. MHC class II deficiency can be achieved by functional deletion or reduction of MHC-II associated genes including, not being limited to, RFXANK, CIITA, RFX5 and RFXAP. CIITA is a transcriptional coactivator, functioning through activation of the transcription factor RFX5 required for class II protein expression. CIITA null cells are MHC-II deficient. In certain embodiments, one or more of the exogenous polynucleotides are integrated at one or more loci of genes selected from the group consisting of B2M, TAP 1, TAP 2, Tapasin, RFXANK, CIITA, RFX5 and RFXAP genes to thereby delete or reduce the expression of the gene(s) with the integration.

Other genes for target deletion include, but are not limited to, recombination-activating genes 1 and 2 (RAG1 and RAG2). RAG1 and RAG2 encode parts of a protein complex that initiate V(D)J recombination by introducing double-strand breaks at the border between a recombination signal sequence (RSS) and a coding segment. Deletion or reducing the expression level of the RAG1/RAG2 genes prevents additional TCR rearrangement in the cell, thus preventing unexpected generation of auto-reactive TCR (Minagawa et al., Cell Stem Cell. 2018 Dec. 6; 23(6):850-858).

In certain embodiments, the exogenous polynucleotides are integrated at one or more loci on the chromosome of the cell, preferably the one or more loci are of genes selected from the group consisting of AAVS1, CCR5, ROSA26, collagen, HTRP, Hl 1, GAPDH, RUNX1, B2M, TAPI, TAP2, Tapasin, NLRC5, CIITA, RFXANK, CIITA, RFX5, RFXAP, TRAC, TRBC1, TRBC2, RAG1, RAG2, NKG2A, NKG2D, CD38, CIS, CBL-B, SOCS2, PD1, CTLA4, LAG3, TIM3, or TIGIT genes, provided at least one of the one or more loci is of a MHC gene, such as a gene selected from the group consisting of B2M, TAP 1, TAP 2, Tapasin, RFXANK, CIITA, RFX5 and RFXAP genes. Preferably, the one or more exogenous polynucleotides are integrated at a locus of an MHC class-I associated gene, such as a beta-2 microglobulin (B2M) gene, TAP 1 gene, TAP 2 gene or Tapasin gene; and at a locus of an MHC-II associated gene, such as a RFXANK, CIITA, RFX5, RFXAP, or CIITA gene; and optionally further at a locus of a safe harbor gene selected from the group consisting of AAVS1, CCR5, ROSA26, collagen, HTRP, Hll, GAPDH, TCR and RUNX1 genes. More preferably, the one or more of the exogenous polynucleotides are integrated at the loci of CIITA, AAVS1 and B2M genes.

In certain embodiments, the exogenous polypeptide encoding the combined artificial cell death/reporter system polypeptide is integrated at a locus of CIITA gene, wherein integration of the exogenous polynucleotide deletes or reduces expression of CIITA gene.

### VII. Derivative Cells

In another aspect, the invention relates to a cell derived from differentiation of an iPSC, a derivative cell. As described above, the genomic edits introduced into the iPSC cell are retained in the derivative cell. In certain embodiments of the derivative cell obtained from iPSC differentiation, the derivative cell is a hematopoietic cell, including, but not limited to, HSCs (hematopoietic stem and progenitor cells), hematopoietic multipotent progenitor cells, T cell progenitors, NK cell progenitors, T cells, NKT cells, NK cells, B cells, antigen presenting cells (APC), monocytes and macrophages. In certain embodiments, the derivative cell is an immune effector cell, such as a NK cell or a T cell.

An iPSC of the application can be differentiated by any method known in the art. Exemplary methods are described in U.S. Pat. Nos. 8,846,395, 8,945,922, 8,318,491, WO2010/099539, WO2012/109208, WO2017/070333, WO2017/179720, WO2016/010148, WO2018/048828 and WO2019/157597, each of which are herein incorporated by reference in its entirety. The differentiation protocol can use feeder cells or can be feeder-free. As used herein, “feeder cells” or “feeders” are terms describing cells of one type that are co-cultured with cells of a second type to provide an environment in which the cells of the second type can grow, expand, or differentiate, as the feeder cells provide stimulation, growth factors and nutrients for the support of the second cell type.

In certain embodiments, the application provides a CD34+ hematopoietic progenitor cell (HPC), a T cell or a natural killer (NK) cell comprising an exogenous polynucleotide encoding an artificial cell polypeptide according to embodiments of the application.

### VIII. Polynucleotides, Vectors, and Host Cells

### (1) Nucleic Acids Encoding a CAR

In another general aspect, the invention relates to an isolated nucleic acid encoding a chimeric antigen receptor (CAR) useful for an invention according to embodiments of the application. It will be appreciated by those skilled in the art that the coding sequence of a CAR can be changed (e.g., replaced, deleted, inserted, etc.) without changing the amino acid sequence of the protein. Accordingly, it will be understood by those skilled in the art that nucleic acid sequences encoding CARs of the application can be altered without changing the amino acid sequences of the proteins.

In certain embodiments, the isolated nucleic acid encodes a CAR targeting CD19. In a particular embodiment, the isolated nucleic acid encoding the CAR comprises a polynucleotide sequence at least 90%, such as at least 90%, 91%, 92%, 93%, 94%, 95%, 96%, 97%, 98% or 100%, identical to SEQ ID NO: 62, preferably the polynucleotide sequence of SEQ ID NO: 62.

In another general aspect, the application provides a vector comprising a polynucleotide sequence encoding a CAR useful for an invention according to embodiments of the application. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of a CAR in the cell. Conventional cloning techniques or artificial gene synthesis can be used to generate a recombinant expression vector according to embodiments of the application.

In a particular aspect, the application provides vectors for targeted integration of a CAR useful for an invention according to embodiments of the application. In certain embodiments, the vector comprises an exogenous polynucleotide having, in the 5′ to 3′ order, (a) a promoter; (b) a polynucleotide sequence encoding a CAR according to an embodiment of the application; and (c) a terminator/polyadenylation signal.

In certain embodiments, the promoter is a CAG promoter. In certain embodiments, the CAG promoter comprises the polynucleotide sequence at least 90%, such as at least 90%, 91%, 92%, 93%, 94%, 95%, 96%, 97%, 98% or 100%, identical to SEQ ID NO: 63. Other promoters can also be used, examples of which include, but are not limited to, EFla, UBC, CMV, SV40, PGK1, and human beta actin.

In certain embodiments, the terminator/polyadenylation signal is a SV40 signal. In certain embodiments, the SV40 signal comprises the polynucleotide sequence at least 90%, such as at least 90%, 91%, 92%, 93%, 94%, 95%, 96%, 97%, 98% or 100%, identical to SEQ ID NO: 64. Other terminator sequences can also be used, examples of which include, but are not limited to, BGH, hGH, and PGK.

In certain embodiments, the polynucleotide sequence encoding a CAR comprises the polynucleotide sequence at least 90%, such as at least 90%, 91%, 92%, 93%, 94%, 95%, 96%, 97%, 98% or 100%, identical to SEQ ID NO: 62.

In some embodiment, the vector further comprises a left homology arm and a right homology arm flanking the exogenous polynucleotide. As used herein, “left homology arm” and “right homology arm” refers to a pair of nucleic acid sequences that flank an exogenous polynucleotide and facilitate the integration of the exogenous polynucleotide into a specified chromosomal locus. Sequences of the left and right arm homology arms can be designed based on the integration site of interest. In some embodiment, the left or right arm homology arm is homologous to the left or right side sequence of the integration site.

In certain embodiments, the left homology arm comprises the polynucleotide sequence at least 90%, such as at least 90%, 91%, 92%, 93%, 94%, 95%, 96%, 97%, 98% or 100%, identical to SEQ ID NO: 84. In certain embodiments, the right homology arm comprises the polynucleotide sequence at least 90%, such as at least 90%, 91%, 92%, 93%, 94%, 95%, 96%, 97%, 98% or 100%, identical to SEQ ID NO: 85.

In a particular embodiment, the vector comprises a polynucleotide sequence at least 85%, such as at least 85%, 86%, 87%, 88%, 89%, 90%, 91%, 92%, 93%, 94%, 95%, 96%, 97%, 98% or 100%, identical to SEQ ID NO: 86, preferably the polynucleotide sequence of SEQ ID NO: 86.

### (2) Nucleic Acids Encoding a Combined Artificial Cell Death/Reporter System Polypeptide

In another general aspect, the invention relates to an isolated nucleic acid encoding a combined artificial cell death/reporter polypeptide according to embodiments of the application. It will be appreciated by those skilled in the art that the coding sequence of a combined artificial cell death/reporter polypeptide can be changed (e.g., replaced, deleted, inserted, etc.) without changing the amino acid sequence of the protein. Accordingly, it will be understood by those skilled in the art that nucleic acid sequences encoding a combined artificial cell death/reporter system polypeptide of the application can be altered without changing the amino acid sequences of the proteins.

In certain embodiments, an isolated nucleic acid encodes any combined artificial cell death/reporter system polypeptide described herein, such as that comprising a herpes simplex virus thymidine kinase (HSV-tk) fused to a prostate-specific membrane antigen (PSMA) polypeptide, optionally via a linker. In certain embodiments, the artificial cell death/reporter system polypeptide consists of an amino acid sequence at least 90%, such as at least 90%, 91%, 82%, 93%, 94%, 95%, 96%, 97%, 98%, 99% or 100%, identical to SEQ ID NOs: 73, 76, 93, or 94. In certain embodiments, the artificial cell death/reporter system polypeptide is encoded by a polynucleotide sequence having at least 90%, 91%, 92%, 93%, 94%, 95%, 96%, 97%, 98%, 99% or 100% sequence identity to a sequence selected from the group consisting of SEQ ID NOs: 74, 77, and 95.

In certain embodiments, the isolated nucleic acid encodes a combined artificial cell death/reporter system polypeptide comprising a herpes simplex virus thymidine kinase (HSV-tk) and a prostate-specific membrane antigen (PSMA) polypeptide operably linked by an autoprotease peptide sequence.

In certain embodiments, the isolated nucleic acid encodes a combined artificial cell death/reporter system polypeptide comprising a prostate-specific membrane antigen (PSMA) polypeptide and a cluster of differentiation 24 (CD24) polypeptide operably linked by an autoprotease peptide sequence. In certain embodiments, the CD24 polypeptide consists of an amino acid sequence at least 90%, such as at least 90%, 91%, 82%, 93%, 94%, 95%, 96%, 97%, 98%, 99% or 100%, identical to SEQ ID NO: 78. In certain embodiments, the artificial cell death/reporter system polypeptide comprising a prostate-specific membrane antigen (PSMA) polypeptide and a cluster of differentiation 24 (CD24) polypeptide consists of an amino acid sequence at least 90%, such as at least 90%, 91%, 82%, 93%, 94%, 95%, 96%, 97%, 98%, 99% or 100%, identical to SEQ ID NO: 79. In certain embodiments, the artificial cell death/reporter system polypeptide comprising a prostate-specific membrane antigen (PSMA) polypeptide and a cluster of differentiation 24 (CD24) polypeptide is encoded by a polynucleotide sequence having at least 90%, 91%, 92%, 93%, 94%, 95%, 96%, 97%, 98%, 99% or 100% sequence identity to SEQ ID NO: 80.

In certain embodiments, the isolated nucleic acid encodes a combined artificial cell death/reporter system polypeptide comprising a herpes simplex virus thymidine kinase (HSV-tk) polypeptide and a cluster of differentiation 52 (CD52) polypeptide operably linked by an autoprotease peptide sequence. In certain embodiments, the CD52 polypeptide consists of an amino acid sequence at least 90%, such as at least 90%, 91%, 82%, 93%, 94%, 95%, 96%, 97%, 98%, 99% or 100%, identical to SEQ ID NO: 91. In certain embodiments, the CD52 polypeptide is encoded by a polynucleotide sequence having at least 90%, 91%, 92%, 93%, 94%, 95%, 96%, 97%, 98%, 99% or 100% sequence identity to SEQ ID NO: 92. In certain embodiments, the artificial cell death/reporter system polypeptide comprising a herpes simplex virus thymidine kinase (HSV-tk) polypeptide and a cluster of differentiation 52 (CD52) polypeptide consists of an amino acid sequence at least 90%, such as at least 90%, 91%, 82%, 93%, 94%, 95%, 96%, 97%, 98%, 99% or 100%, identical to SEQ ID NO: 96 or 97. In certain embodiments, the artificial cell death/reporter system polypeptide comprising a herpes simplex virus thymidine kinase (HSV-tk) polypeptide and a cluster of differentiation 52 (CD52) polypeptide consists of an amino acid sequence at least 90%, such as at least 90%, 91%, 82%, 93%, 94%, 95%, 96%, 97%, 98%, 99% or 100%, identical to SEQ ID NO: 98.

In certain embodiments, the HSV-tk consists of an amino acid sequence at least 90%, such as at least 90%, 91%, 82%, 93%, 94%, 95%, 96%, 97%, 98%, 99% or 100%, identical to SEQ ID NO: 71 or 89. In certain embodiments, the HSV-tk polypeptide is encoded by a polynucleotide sequence having at least 90%, 91%, 92%, 93%, 94%, 95%, 96%, 97%, 98%, 99% or 100% sequence identity to SEQ ID NO: 90.

In certain embodiments, the PSMA polypeptide consists of an amino acid sequence at least 90%, such as at least 90%, 91%, 82%, 93%, 94%, 95%, 96%, 97%, 98%, 99% or 100%, identical to SEQ ID NO: 72. In some embodiments, the PSMA polypeptide consists of an N9del variant of PSMA.

In certain embodiments, the linker consists of an amino acid sequence of SEQ ID NO: 48.

In certain embodiments, the autoprotease peptide is a thosea asigna virus 2A (T2A) peptide having an amino acid of SEQ ID NO: 75 or 87.

In certain embodiments, the CD24 consists of an amino acid sequence at least 90%, such as at least 90%, 91%, 82%, 93%, 94%, 95%, 96%, 97%, 98%, 99% or 100%, identical to SEQ ID NO: 78.

In certain embodiments, the isolated nucleic acid encoding the combined artificial cell death/reporter system polypeptide comprises a polynucleotide sequence at least 90%, such as at least 90%, 91%, 92%, 93%, 94%, 95%, 96%, 97%, 98% or 100%, identical to SEQ ID NO: 74, preferably the polynucleotide sequence of SEQ ID NO: 74.

In certain embodiments, the isolated nucleic acid encoding the combined artificial cell death/reporter system polypeptide comprises a polynucleotide sequence at least 90%, such as at least 90%, 91%, 92%, 93%, 94%, 95%, 96%, 97%, 98% or 100%, identical to SEQ ID NO: 77, preferably the polynucleotide sequence of SEQ ID NO: 77.

In certain embodiments, the isolated nucleic acid encoding the combined artificial cell death/reporter system polypeptide comprises a polynucleotide sequence at least 90%, such as at least 90%, 91%, 92%, 93%, 94%, 95%, 96%, 97%, 98% or 100%, identical to SEQ ID NO: 80, preferably the polynucleotide sequence of SEQ ID NO: 80.

In another general aspect, the application provides a vector comprising a polynucleotide sequence encoding a combined artificial cell death/reporter system polypeptide according to embodiments of the application. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of an inactivated cell surface receptor in the cell. Conventional cloning techniques or artificial gene synthesis can be used to generate a recombinant expression vector according to embodiments of the application.

In a particular aspect, the application provides a vector for expression of a combined artificial cell death/reporter system polypeptide according to embodiments of the application. In certain embodiments, the vector comprises an exogenous polynucleotide having, in the 5′ to 3′ order, (a) a promoter; (b) a polynucleotide sequence encoding a combined artificial cell death/reporter system polypeptide, such as a coding sequence of a herpes simplex virus thymidine kinase (HSV-tk), a coding sequence of a linker, and a coding sequence of a prostate-specific membrane antigen (PSMA) polypeptide, and (c) a terminator/polyadenylation signal.

In certain embodiments, the promoter is a CAG promoter. In certain embodiments, the CAG promoter comprises the polynucleotide sequence at least 90%, such as at least 90%, 91%, 92%, 93%, 94%, 95%, 96%, 97%, 98% or 100%, identical to SEQ ID NO: 63. Other promoters can also be used, examples of which include, but are not limited to, EFla, UBC, CMV, SV40, PGK1, and human beta actin.

In certain embodiments, the terminator/polyadenylation signal is a SV40 signal. In certain embodiments, the SV40 signal comprises the polynucleotide sequence at least 90%, such as at least 90%, 91%, 92%, 93%, 94%, 95%, 96%, 97%, 98% or 100%, identical to SEQ ID NO: 64. Other terminator sequences can also be used, examples of which include, but are not limited to BGH, hGH, and PGK.

In some embodiment, the vector further comprises a left homology arm and a right homology arm flanking the exogenous polynucleotide.

TABLE 2
Nucleic acids encoding artificial cell death/reporter system

| | | | SEQ |
|---|---|---|---|
| | Sequence | | ID NO |
| HSV | MASYPCHQHA SAFDQAARSR GHSNRRTALR PRRQQEATEV RLEQKMPTLL RVYIDGPHGM | 60 | 71 |
| Thymidine | GKTTTTQLLV ALGSRDDIVY VPEPMTYWQV LGASETIANI YTTQHRLDQG EISAGDAAVV | 120 | |
| Kinase | MTSAQITMGM PYAVTDAVLA PHIGGEAGSS HAPPPALTLI FDRHPIAALL CYPAARYLMG | 180 | |
| (amino acid) | SMTPQAVLAF VALIPPTLPG TNIVLGALPE DRHIDRLAKR QRPGERLDLA MLAAIRRVYG | 240 | |
| | LLANTVRYLQ GGGSWREDWG QLSGTAVPPQ GAEPQSNAGP RPHIGDTLFT LFRAPELLAP | 300 | |
| | NGDLYNVFAW ALDVLAKRLR PMHVFILDYD QSPAGCRDAL LQLTSGMVQT HVTTPGSIPT | 360 | |
| | ICDLARTFAR EMGEAN | 376 | |
| PSMA | MWNLLARRPR WLCAGALVLA GGFFLLGFLF GWFIKSSNEA TNITPKHNMK AFLDELKAEN | 60 | 72 |
| Variant | IKKFLYNFTQ IPHLAGTEQN FQLAKQIQSQ WKEFGLDSVE LAHYDVLLSY PNKTHPNYIS | 120 | |
| (amino acid) | IINEDGNEIF NTSLFEPPPP GYENVSDIVP PFSAFSPQGM PEGDLVYVNY ARTEDFFKLE | 180 | |
| | RDMKINCSGK IVIARYGKVF RGNKVKNAQL AGAKGVILYS DPADYFAPGV KSYPDGWNLP | 240 | |
| | GGGVQRGNIL NLNGAGDPLT PGYPANEYAY RRGIAEAVGL PSIPVHPIGY YDAQKLLEKM | 300 | |
| | GGSAPPDSSW RGSLKVPYNV GPGFTGNFST QKVKMHIHST NEVTRIYNVI GTLRGAVEPD | 360 | |
| | RYVILGGHRD SWVFGGIDPQ SGAAVVHEIV RSFGTLKKEG WRPRRTILFA SWDAEEFGLL | 420 | |
| | GSTEWAEENS RLLQERGVAY INADSSIEGN YTLRVDCTPL MYSLVHNLTK ELKSPDEGFE | 480 | |
| | GKSLYESWTK KSPSPEFSGM PRISKLGSGN DFEVFFQRLG IASGRARYTK NWETNKFSGY | 540 | |
| | PLYHSVYETY ELVEKFYDPM FKYHLTVAQV RGGMVFELAN SIVLPFDCRD YAVVLRKYAD | 600 | |
| | KIYSISMKHP QEMKTYSVSF DSLFSAVKNF TEIASKFSER LQDFDKSNPI VLRMMNDQLM | 660 | |
| | FLERAFIDPL GLPDRPFYRH VIYAPSSHNK YAGESFPGIY DALFDIESKV DPSKAWGEVK | 720 | |
| | RQIYVAAFTV QAAAETLSEV A | 741 | |
| HSV-tk- | MASYPCHQHA SAFDQAARSR GHSNRRTALR PRRQQEATEV RLEQKMPTLL RVYIDGPHGM | 60 | 73 |
| PSMA | GKTTTTQLLV ALGSRDDIVY VPEPMTYWQV LGASETIANI YTTQHRLDQG EISAGDAAVV | 120 | |
| Fusion | MTSAQITMGM PYAVTDAVLA PHIGGEAGSS HAPPPALTLI FDRHPIAALL CYPAARYLMG | 180 | |
| (amino acid) | SMTPQAVLAF VALIPPTLPG TNIVLGALPE DRHIDRLAKR QRPGERLDLA MLAAIRRVYG | 240 | |
| | LLANTVRYLQ GGGSWREDWG QLSGTAVPPQ GAEPQSNAGP RPHIGDTLFT LFRAPELLAP | 300 | |
| | NGDLYNVFAW ALDVLAKRLR PMHVFILDYD QSPAGCRDAL LQLTSGMVQT HVTTPGSIPT | 360 | |
| | ICDLARTFAR EMGEANGSTS GSGKPGSGEG STKGMWNLLA RRPRWLCAGA LVLAGGFFLL | 420 | |
| | GFLFGWFIKS SNEATNITPK HNMKAFLDEL KAENIKKFLY NFTQIPHLAG TEQNFQLAKQ | 480 | |
| | IQSQWKEFGL DSVELAHYDV LLSYPNKTHP NYISIINEDG NEIFNTSLFE PPPPGYENVS | 540 | |
| | DIVPPFSAFS PQGMPEGDLV YVNYARTEDF FKLERDMKIN CSGKIVIARY GKVFRGNKVK | 600 | |
| | NAQLAGAKGV ILYSDPADYF APGVKSYPDG WNLPGGGVQR GNILNLNGAG DPLTPGYPAN | 660 | |
| | EYAYRRGIAE AVGLPSIPVH PIGYYDAQKL LEKMGGSAPP DSSWRGSLKV PYNVGPGFTG | 720 | |
| | NFSTQKVKMH IHSTNEVTRI YNVIGTLRGA VEPDRYVILG GHRDSWVFGG IDPQSGAAVV | 780 | |
| | HEIVRSFGTL KKEGWRPRRT ILFASWDAEE FGLLGSTEWA EENSRLLQER GVAYINADSS | 840 | |
| | IEGNYTLRVD CTPLMYSLVH NLTKELKSPD EGFEGKSLYE SWTKKSPSPE FSGMPRISKL | 900 | |
| | GSGNDFEVFF QRLGIASGRA RYTKNWETNK FSGYPLYHSV YETYELVEKF YDPMFKYHLT | 960 | |
| | VAQVRGGMVF ELANSIVLPF DCRDYAVVLR KYADKIYSIS MKHPQEMKTY SVSFDSLFSA | 1020 | |
| | VKNFTEIASK FSERLQDFDK SNPIVLRMMN DQLMFLERAF IDPLGLPDRP FYRHVIYAPS | 1080 | |
| | SHNKYAGESF PGIYDALFDI ESKVDPSKAW GEVKRQIYVA AFTVQAAAET LSEVA | 1135 | |
| HSV-tk- | ATGGCCTCAT ATCCCTGCCA CCAGCATGCG AGTGCGTTTG ATCAAGCTGC ACGGTCAAGA | 60 | 74 |
| PSMA | GGACATTCCA ATAGGCGAAC TGCACTGAGA CCTAGGCGAC AACAAGAAGC AACGGAGGTC | 120 | |
| Fusion | CGCCTTGAGC AGAAGATGCC TACTCTTCTT AGGGTTTACA TCGATGGTCC GCACGGAATG | 180 | |
| (nucleic acid) | GGTAAGACCA CGACTACTCA ACTGCTTGTC GCATTGGGCT CCAGGGATGA CATAGTTTAC | 240 | |
| | GTTCCGGAAC CTATGACTTA TTGGCAGGTG TTGGGGGCAT CCGAAACCAT AGCTAATATT | 300 | |
| | TATACCACGC AACACCGCTT GGACCAAGGA GAGATCTCTG CGGGAGATGC AGCGGTGGTA | 360 | |
| | ATGACCAGCG CACAAATCAC CATGGGGATG CCGTATGCCG TAACAGACGC GGTGCTGGCC | 420 | |
| | CCCCATATCG GTGGTGAAGC CGGTTCAAGT CATGCGCCTC CGCCGGCGCT GACATTGATC | 480 | |
| | TTTGATCGGC ATCCTATTGC GGCACTCCTG TGTTATCCAG CCGCACGCTA TCTGATGGGA | 540 | |
| | AGCATGACCC CACAAGCGGT TCTGGCCTTT GTGGCATTGA TCCCACCAAC ATTGCCTGGA | 600 | |
| | ACTAATATTG TACTGGGCGC TCTCCCCGAG GATCGACATA TTGATAGGCT CGCGAAACGC | 660 | |
| | CAACGCCCTG GTGAAAGGCT TGATTTGGCC ATGCTCGCGG CTATACGCCG CGTCTATGGG | 720 | |
| | TTGCTCGCCA ATACTGTGAG ATACCTGCAG GGCGGGGGGT CATGGCGCGA GGATTGGGGT | 780 | |
| | CAGTTGTCAG GCACGGCAGT GCCCCCACAG GGGGCGGAGC CTCAGTCAAA TGCAGGCCCA | 840 | |
| | AGACCGCATA TTGGTGATAC TCTCTTCACG TTGTTTCGGG CGCCAGAATT GCTTGCCCCC | 900 | |
| | AACGGTGATC TTTATAACGT CTTCGCATGG GCCCTCGATG TCTTGGCCAA GAGGCTGCGA | 960 | |
| | CCGATGCACG TTTTTATTCT CGACTATGAC CAGTCACCTG CGGGATGCAG GGATGCGTTG | 1020 | |
| | CTCCAGTTGA CAAGTGGCAT GGTGCAGACA CATGTGACGA CACCCGGATC AATACCTACG | 1080 | |
| | ATATGTGATC TCGCAAGGAC CTTTGCGAGG GAAATGGGGG AAGCTAATGG CTCAACTTCT | 1140 | |
| | GGATCCGGAA AGCCCGGTAG CGGCGAAGGT TCTACAAAGG GTATGTGGAA CCTGTTGGCA | 1200 | |
| | AGACGCCCCC GCTGGCTCTG TGCCGGTGCT CTGGTACTGG CGGGGGGATT TTTTCTCTTG | 1260 | |
| | GGATTTCTTT TTGGGTGGTT CATAAAAAGT TCTAATGAGG CCACAAATAT CACTCCGAAA | 1320 | |
| | CACAATATGA AGGCATTTCT GGACGAGCTC AAAGCGGAAA ATATTAAGAA ATTCCTGTAT | 1380 | |
| | AACTTTACTC AGATACCTCA TCTGGCTGGC ACCGAGCAAA ACTTCCAGTT GGCTAAGCAG | 1440 | |
| | ATTCAATCAC AGTGGAAAGA GTTCGGTCTC GATAGCGTTG AATTGGCACA CTACGATGTC | 1500 | |
| | CTTCTTAGCT ATCCTAATAA AACACATCCG AACTACATAA GTATCATTAA TGAGGACGGC | 1560 | |
| | AACGAAATTT TCAACACCTC TCTTTTTGAA CCTCCTCCGC CGGGATATGA AAACGTGAGC | 1620 | |
| | GATATCGTTC CCCCCTTTTC CGCGTTCTCA CCACAGGGAA TGCCGGAGGG TGACCTTGTT | 1680 | |
| | TATGTAAATT ATGCCAGAAC GGAAGATTTC TTTAAACTTG AACGCGATAT GAAAATCAAC | 1740 | |
| | TGCTCTGGTA AGATTGTTAT TGCCCGATAC GGGAAGGTAT TCAGGGGTAA CAAAGTGAAA | 1800 | |
| | AACGCTCAGC TGGCGGGTGC CAAAGGAGTG ATCCTTTATT CTGACCCTGC GGATTATTTC | 1860 | |
| | GCACCAGGCG TAAAATCATA TCCTGACGGT TGGAACCTTC CTGGAGGTGG AGTACAGCGC | 1920 | |
| | GGAAATATAT TGAATCTTAA CGGAGCCGGT GACCCACTGA CTCCTGGATA CCCCGCAAAC | 1980 | |
| | GAGTATGCCT ATCGACGCGG CATTGCCGAA GCGGTGGGAC TGCCCTCAAT ACCTGTACAT | 2040 | |
| | CCTATTGGAT ACTATGATGC TCAGAAACTT TTGGAGAAGA TGGGTGGAAG TGCCCCGCCT | 2100 | |
| | GATAGTTCCT GGAGAGGCTC CCTTAAGGTT CCATATAACG TAGGTCCAGG GTTTACGGGC | 2160 | |
| | AACTTTTCAA CACAAAAGGT AAAGATGCAT ATACATTCAA CTAACGAGGT GACGAGGATA | 2220 | |
| | TACAATGTAA TCGGAACTCT GAGGGGAGCC GTAGAGCCTG ATCGATATGT CATCTTGGGG | 2280 | |
| | GGCCACAGGG ATAGTTGGGT CTTTGGTGGA ATTGATCCTC AGTCAGGTGC GGCTGTAGTC | 2340 | |
| | CACGAGATTG TCCGCTCTTT CGGCACGCTG AAGAAGGAGG GGTGGAGACC CCGAAGGACT | 2400 | |
| | ATTTTGTTTG CCTCTTGGGA TGCTGAAGAA TTTGGTCTGC TCGGATCAAC GGAGTGGGCT | 2460 | |
| | GAAGAGAATT CTAGGTTGTT GCAAGAACGC GGTGTGGCCT ACATAAATGC GGACAGTAGT | 2520 | |
| | ATAGAAGGCA ATTACACACT TCGAGTGGAT TGCACCCCGC TTATGTACAG TCTGGTACAT | 2580 | |
| | AACCTGACGA AGGAGCTTAA ATCACCTGAT GAAGGATTCG AGGGGAAATC CCTTTACGAA | 2640 | |
| | TCATGGACTA AAAAGTCACC TTCCCCTGAA TTTAGTGGGA TGCCGCGCAT AAGTAAACTC | 2700 | |
| | GGGTCCGGAA ACGACTTCGA AGTTTTCTTC CAACGATTGG GTATCGCCTC TGGACGAGCA | 2760 | |
| | CGGTACACCA AAAATTGGGA AACGAACAAA TTTTCCGGAT ATCCTCTCTA CCACTCTGTC | 2820 | |
| | TATGAAACCT ACGAGCTGGT GGAAAAGTTT TACGATCCGA TGTTTAAGTA CCATTTGACC | 2880 | |
| | GTCGCCCAGG TGCGGGGAGG AATGGTCTTT GAATTGGCAA ATAGTATAGT CCTTCCTTTT | 2940 | |
| | GATTGTCGAG ATTATGCCGT CGTCCTTAGG AAGTATGCTG ACAAGATTTA TTCAATATCT | 3000 | |
| | ATGAAGCACC CCCAAGAAAT GAAGACCTAC TCAGTGTCCT TTGACTCCCT GTTCTCCGCT | 3060 | |
| | GTGAAGAACT TCACTGAGAT CGCCTCTAAG TTCTCAGAGC GACTGCAAGA TTTTGACAAG | 3120 | |
| | AGTAACCCCA TTGTTCTTAG GATGATGAAT GACCAGCTCA TGTTTTTGGA GAGAGCATTT | 3180 | |
| | ATTGATCCGC TGGGCCTTCC CGACCGGCCA TTTTACAGAC ACGTTATCTA TGCTCCTTCA | 3240 | |
| | AGTCACAATA AATATGCAGG AGAATCCTTT CCTGGGATCT ACGATGCCCT CTTCGACATA | 3300 | |
| | GAAAGTAAGG TTGATCCCTC CAAGGCGTGG GGGGAAGTTA AACGACAGAT ATATGTCGCT | 3360 | |
| | GCATTCACCG TACAGGCCGC CGCAGAGACA CTTAGTGAGG TTGCGTGA | 3408 | |
| T2A (amino | SGSGEGRGSL LTCGDVEENP GP | 22 | 75 |
| acid) | | | |
| HSV-tk- | MASYPCHQHA SAFDQAARSR GHSNRRTALR PRRQQEATEV RLEQKMPTLL RVYIDGPHGM | 60 | 76 |
| T2A-PSMA | GKTTTTQLLV ALGSRDDIVY VPEPMTYWQV LGASETIANI YTTQHRLDQG EISAGDAAVV | 120 | |
| Fusion | MTSAQITMGM PYAVTDAVLA PHIGGEAGSS HAPPPALTLI FDRHPIAALL CYPAARYLMG | 180 | |
| (amino acid) | SMTPQAVLAF VALIPPTLPG TNIVLGALPE DRHIDRLAKR QRPGERLDLA MLAAIRRVYG | 240 | |
| | LLANTVRYLQ GGGSWREDWG QLSGTAVPPQ GAEPQSNAGP RPHIGDTLFT LFRAPELLAP | 300 | |
| | NGDLYNVFAW ALDVLAKRLR PMHVFILDYD QSPAGCRDAL LQLTSGMVQT HVTTPGSIPT | 360 | |
| | ICDLARTFAR EMGEANSGSG EGRGSLLTCG DVEENPGPMW NLLARRPRWL CAGALVLAGG | 420 | |
| | FFLLGFLFGW FIKSSNEATN ITPKHNMKAF LDELKAENIK KFLYNFTQIP HLAGTEQNFQ | 480 | |
| | LAKQIQSQWK EFGLDSVELA HYDVLLSYPN KTHPNYISII NEDGNEIFNT SLFEPPPPGY | 540 | |
| | ENVSDIVPPF SAFSPQGMPE GDLVYVNYAR TEDFFKLERD MKINCSGKIV IARYGKVFRG | 600 | |
| | NKVKNAQLAG AKGVILYSDP ADYFAPGVKS YPDGWNLPGG GVQRGNILNL NGAGDPLTPG | 660 | |
| | YPANEYAYRR GIAEAVGLPS IPVHPIGYYD AQKLLEKMGG SAPPDSSWRG SLKVPYNVGP | 720 | |
| | GFTGNFSTQK VKMHIHSTNE VTRIYNVIGT LRGAVEPDRY VILGGHRDSW VFGGIDPQSG | 780 | |
| | AAVVHEIVRS FGTLKKEGWR PRRTILFASW DAEEFGLLGS TEWAEENSRL LQERGVAYIN | 840 | |
| | ADSSIEGNYT LRVDCTPLMY SLVHNLTKEL KSPDEGFEGK SLYESWTKKS PSPEFSGMPR | 900 | |
| | ISKLGSGNDF EVFFQRLGIA SGRARYTKNW ETNKFSGYPL YHSVYETYEL VEKFYDPMFK | 960 | |
| | YHLTVAQVRG GMVFELANSI VLPFDCRDYA VVLRKYADKI YSISMKHPQE MKTYSVSFDS | 1020 | |
| | LFSAVKNFTE IASKFSERLQ DFDKSNPIVL RMMNDQLMFL ERAFIDPLGL PDRPFYRHVI | 1080 | |
| | YAPSSHNKYA GESFPGIYDA LFDIESKVDP SKAWGEVKRQ IYVAAFTVQA AAETLSEVA | 1139 | |
| HSV-tk- | ATGGCAAGTT ACCCTTGCCA CCAGCATGCT AGCGCTTTCG ATCAAGCGGC CCGCAGTCGG | 60 | 77 |
| T2A-PSMA | GGCCATAGCA ATAGAAGGAC CGCATTGCGC CCGCGCCGAC AGGAGGAAGC TACCGAAGTA | 120 | |
| Fusion | CGACTGGAAC AAAAAATGCC CACTCTTCTG AGAGTATACA TCGATGGGCC ACACGGCATG | 180 | |
| (nucleic acid) | GGCAAAACGA CGACTACCCA ACTCTTGGTA GCTCTTGGTA GCCGGGATGA TATCGTATAT | 240 | |
| | GTGCCAGAAC CTATGACCTA TTGGCAGGTC CTCGGCGCCA GTGAAACCAT CGCCAACATA | 300 | |
| | TATACGACGC AACATAGGCT TGACCAGGGT GAAATCTCCG CAGGTGACGC GGCAGTGGTC | 360 | |
| | ATGACTAGCG CCCAAATCAC GATGGGAATG CCTTATGCGG TGACTGACGC AGTACTCGCT | 420 | |
| | CCTCACATTG GAGGTGAAGC GGGGAGCTCC CACGCACCGC CGCCCGCTCT TACGCTCATT | 480 | |
| | TTCGATCGCC ACCCTATAGC TGCCCTGCTC TGTTATCCCG CGGCCAGGTA CTTGATGGGG | 540 | |
| | TCCATGACCC CCCAGGCGGT GCTGGCCTTC GTTGCGTTGA TACCGCCAAC TCTCCCCGGC | 600 | |
| | ACTAATATTG TTCTCGGAGC ACTTCCAGAA GACAGGCATA TTGACAGGTT GGCTAAGCGC | 660 | |
| | CAAAGGCCCG GTGAACGACT CGACCTGGCT ATGCTTGCTG CCATCCGCCG CGTCTATGGG | 720 | |
| | CTGCTGGCTA ATACGGTCAG GTATCTTCAA GGCGGCGGAT CTTGGAGGGA AGATTGGGGA | 780 | |
| | CAGCTCAGTG GAACGGCTGT ACCTCCACAA GGGGCCGAAC CTCAGTCAAA TGCAGGTCCT | 840 | |
| | CGCCCTCACA TTGGAGATAC ACTTTTTACT CTTTTCCGGG CTCCAGAACT GCTGGCACCA | 900 | |
| | AATGGCGACC TGTACAATGT TTTTGCCTGG GCTCTGGATG TTTTGGCAAA AAGGCTTCGG | 960 | |
| | CCGATGCACG TATTTATTTT GGACTATGAT CAGTCACCGG CTGGTTGTAG AGACGCATTG | 1020 | |
| | CTTCAACTTA CATCCGGGAT GGTACAAACG CATGTAACAA CCCCAGGGTC AATTCCAACT | 1080 | |
| | ATCTGCGATC TCGCCCGCAC ATTCGCAAGA GAAATGGGTG AGGCTAACTC TGGCAGTGGT | 1140 | |
| | GAAGGCCGCG GATCTCTCCT GACTTGTGGG GATGTCGAAG AAAACCCGGG ACCCATGTGG | 1200 | |
| | AACCTGTTGG CAAGACGCCC CCGCTGGCTC TGTGCCGGTG CTCTGGTACT GGCGGGGGGA | 1260 | |
| | TTTTTTCTCT TGGGATTTCT TTTTGGGTGG TTCATAAAAA GTTCTAATGA GGCCACAAAT | 1320 | |
| | ATCACTCCGA AACACAATAT GAAGGCATTT CTGGACGAGC TCAAAGCGGA AAATATTAAG | 1380 | |
| | AAATTCCTGT ATAACTTTAC TCAGATACCT CATCTGGCTG GCACCGAGCA AAACTTCCAG | 1440 | |
| | TTGGCTAAGC AGATTCAATC ACAGTGGAAA GAGTTCGGTC TCGATAGCGT TGAATTGGCA | 1500 | |
| | CACTACGATG TCCTTCTTAG CTATCCTAAT AAAACACATC CGAACTACAT AAGTATCATT | 1560 | |
| | AATGAGGACG GCAACGAAAT TTTCAACACC TCTCTTTTTG AACCTCCTCC GCCGGGATAT | 1620 | |
| | GAAAACGTGA GCGATATCGT TCCCCCCTTT TCCGCGTTCT CACCACAGGG AATGCCGGAG | 1680 | |
| | GGTGACCTTG TTTATGTAAA TTATGCCAGA ACGGAAGATT TCTTTAAACT TGAACGCGAT | 1740 | |
| | ATGAAAATCA ACTGCTCTGG TAAGATTGTT ATTGCCCGAT ACGGGAAGGT ATTCAGGGGT | 1800 | |
| | AACAAAGTGA AAAACGCTCA GCTGGCGGGT GCCAAAGGAG TGATCCTTTA TTCTGACCCT | 1860 | |
| | GCGGATTATT TCGCACCAGG CGTAAAATCA TATCCTGACG GTTGGAACCT TCCTGGAGGT | 1920 | |
| | GGAGTACAGC GCGGAAATAT ATTGAATCTT AACGGAGCCG GTGACCCACT GACTCCTGGA | 1980 | |
| | TACCCCGCAA ACGAGTATGC CTATCGACGC GGCATTGCCG AAGCGGTGGG ACTGCCCTCA | 2040 | |
| | ATACCTGTAC ATCCTATTGG ATACTATGAT GCTCAGAAAC TTTTGGAGAA GATGGGTGGA | 2100 | |
| | AGTGCCCCGC CTGATAGTTC CTGGAGAGGC TCCCTTAAGG TTCCATATAA CGTAGGTCCA | 2160 | |
| | GGGTTTACGG GCAACTTTTC AACACAAAAG GTAAAGATGC ATATACATTC AACTAACGAG | 2220 | |
| | GTGACGAGGA TATACAATGT AATCGGAACT CTGAGGGGAG CCGTAGAGCC TGATCGATAT | 2280 | |
| | GTCATCTTGG GGGGCCACAG GGATAGTTGG GTCTTTGGTG GAATTGATCC TCAGTCAGGT | 2340 | |
| | GCGGCTGTAG TCCACGAGAT TGTCCGCTCT TTCGGCACGC TGAAGAAGGA GGGGTGGAGA | 2400 | |
| | CCCCGAAGGA CTATTTTGTT TGCCTCTTGG GATGCTGAAG AATTTGGTCT GCTCGGATCA | 2460 | |
| | ACGGAGTGGG CTGAAGAGAA TTCTAGGTTG TTGCAAGAAC GCGGTGTGGC CTACATAAAT | 2520 | |
| | GCGGACAGTA GTATAGAAGG CAATTACACA CTTCGAGTGG ATTGCACCCC GCTTATGTAC | 2580 | |
| | AGTCTGGTAC ATAACCTGAC GAAGGAGCTT AAATCACCTG ATGAAGGATT CGAGGGGAAA | 2640 | |
| | TCCCTTTACG AATCATGGAC TAAAAAGTCA CCTTCCCCTG AATTTAGTGG GATGCCGCGC | 2700 | |
| | ATAAGTAAAC TCGGGTCCGG AAACGACTTC GAAGTTTTCT TCCAACGATT GGGTATCGCC | 2760 | |
| | TCTGGACGAG CACGGTACAC CAAAAATTGG GAAACGAACA AATTTTCCGG ATATCCTCTC | 2820 | |
| | TACCACTCTG TCTATGAAAC CTACGAGCTG GTGGAAAAGT TTTACGATCC GATGTTTAAG | 2880 | |
| | TACCATTTGA CCGTCGCCCA GGTGCGGGGA GGAATGGTCT TTGAATTGGC AAATAGTATA | 2940 | |
| | GTCCTTCCTT TTGATTGTCG AGATTATGCC GTCGTCCTTA GGAAGTATGC TGACAAGATT | 3000 | |
| | TATTCAATAT CTATGAAGCA CCCCCAAGAA ATGAAGACCT ACTCAGTGTC CTTTGACTCC | 3060 | |
| | CTGTTCTCCG CTGTGAAGAA CTTCACTGAG ATCGCCTCTA AGTTCTCAGA GCGACTGCAA | 3120 | |
| | GATTTTGACA AGAGTAACCC CATTGTTCTT AGGATGATGA ATGACCAGCT CATGTTTTTG | 3180 | |
| | GAGAGAGCAT TTATTGATCC GCTGGGCCTT CCCGACCGGC CATTTTACAG ACACGTTATC | 3240 | |
| | TATGCTCCTT CAAGTCACAA TAAATATGCA GGAGAATCCT TTCCTGGGAT CTACGATGCC | 3300 | |
| | CTCTTCGACA TAGAAAGTAA GGTTGATCCC TCCAAGGCGT GGGGGGAAGT TAAACGACAG | 3360 | |
| | ATATATGTCG CTGCATTCAC CGTACAGGCC GCCGCAGAGA CACTTAGTGA GGTTGCGTGA | 3420 | |
| CD24 (amino | MGRAMVARLG LGLLLLALLL PTQIYSSETT TGTSSNSSQS TSNSGLAPNP TNATTKAAGG | 60 | 78 |
| acid) | ALQSTASLFV VSLSLLHLYS | 80 | |
| PSMA-T2A- | MWNLLARRPR WLCAGALVLA GGFFLLGFLF GWFIKSSNEA TNITPKHNMK AFLDELKAEN | 60 | 79 |
| CD24 Fusion | IKKFLYNFTQ IPHLAGTEQN FQLAKQIQSQ WKEFGLDSVE LAHYDVLLSY PNKTHPNYIS | 120 | |
| (amino acid) | IINEDGNEIF NTSLFEPPPP GYENVSDIVP PFSAFSPQGM PEGDLVYVNY ARTEDFFKLE | 180 | |
| | RDMKINCSGK IVIARYGKVF RGNKVKNAQL AGAKGVILYS DPADYFAPGV KSYPDGWNLP | 240 | |
| | GGGVQRGNIL NLNGAGDPLT PGYPANEYAY RRGIAEAVGL PSIPVHPIGY YDAQKLLEKM | 300 | |
| | GGSAPPDSSW RGSLKVPYNV GPGFTGNFST QKVKMHIHST NEVTRIYNVI GTLRGAVEPD | 360 | |
| | RYVILGGHRD SWVFGGIDPQ SGAAVVHEIV RSFGTLKKEG WRPRRTILFA SWDAEEFGLL | 420 | |
| | GSTEWAEENS RLLQERGVAY INADSSIEGN YTLRVDCTPL MYSLVHNLTK ELKSPDEGFE | 480 | |
| | GKSLYESWTK KSPSPEFSGM PRISKLGSGN DFEVFFQRLG IASGRARYTK NWETNKFSGY | 540 | |
| | PLYHSVYETY ELVEKFYDPM FKYHLTVAQV RGGMVFELAN SIVLPFDCRD YAVVLRKYAD | 600 | |
| | KIYSISMKHP QEMKTYSVSF DSLFSAVKNF TEIASKFSER LQDFDKSNPI VLRMMNDQLM | 660 | |
| | FLERAFIDPL GLPDRPFYRH VIYAPSSHNK YAGESFPGIY DALFDIESKV DPSKAWGEVK | 720 | |
| | RQIYVAAFTV QAAAETLSEV ASGSGEGRGS LLTCGDVEEN PGPMGRAMVA RLGLGLLLLA | 780 | |
| | LLLPTQIYSS ETTTGTSSNS SQSTSNSGLA PNPTNATTKA AGGALQSTAS LFVVSLSLLH | 840 | |
| | LYS | 843 | |
| PSMA-T2A- | ATGTGGAACC TGCTTGCTAG AAGGCCTAGA TGGCTTTGTG CTGGCGCTCT GGTTCTGGCT | 60 | 80 |
| CD24 Fusion | GGCGGCTTTT TTCTGCTGGG CTTCCTGTTC GGCTGGTTCA TCAAGAGCAG CAAGGAGGCC | 120 | |
| (nucleic acid) | ACCAATATCA CCCCTAAGCA CAACATGAAG GCCTTTCTGG ACGAGCTGAA GGCCGAGAAT | 180 | |
| | ATCAAGAAGT TCCTGTACAA CTTCACGCAG ATCCCTCACC TGGCCGGCAC CGAGCAGAAT | 240 | |
| | TTTCAGCTGG CCAAGCAGAT CCAGAGCCAG TGGAAAGAGT TCGGCCTGGA CTCTGTGGAA | 300 | |
| | CTGGCCCACT ACGATGTGCT GCTGAGCTAC CCCAACAAGA CACACCCCAA CTACATCAGC | 360 | |
| | ATCATCAACG AGGACGGCAA CGAGATCTTC AATACCAGCC TGTTCGAGCC TCCTCCACCT | 420 | |
| | GGCTACGAGA ACGTGTCCGA TATCGTGCCT CCATTCAGCG CTTTCAGCCC TCAAGGGATG | 480 | |
| | CCTGAGGGCG ATCTGGTGTA CGTGAACTAC GCCAGAACCG AGGACTTCTT CAAGCTGGAA | 540 | |
| | CGGGACATGA AGATCAACTG CAGCGGCAAG ATCGTGATCG CCAGATACGG CAAGGTGTTC | 600 | |
| | CGGGGCAACA AAGTGAAGAA CGCCCAACTG GCAGGCGCCA AGGGCGTGAT CCTGTATTCT | 660 | |
| | GATCCCGCCG ACTACTTCGC CCCTGGCGTG AAGTCTTATC CCGACGGCTG GAATCTTCCT | 720 | |
| | GGCGGCGGAG TTCAGAGGGG CAATATCCTG AACCTGAACG GCGCTGGCGA CCCTCTGACA | 780 | |
| | CCTGGATATC CTGCCAACGA GTACGCCTAC AGACGGGGAA TTGCCGAAGC TGTGGGCCTG | 840 | |
| | CCTTCTATCC CTGTGCACCC AATCGGCTAC TACGACGCCC AGAAACTGCT GGAAAAGATG | 900 | |
| | GGCGGAAGCG CCCCTCCTGA CTCTTCTTGG AGAGGCTCTC TGAAGGTGCC CTACAATGTC | 960 | |
| | GGCCCTGGCT TCACCGGCAA CTTCAGCACC CAGAAAGTGA AAATGCACAT CCACAGCACC | 1020 | |
| | AACGAAGTGA CCCGGATCTA CAACGTGATC GGCACACTGA GAGGCGCCGT GGAACCCGAC | 1080 | |
| | AGATATGTGA TCCTCGGCGG CCACAGAGAC AGCTGGGTGT TCGGAGGAAT CGACCCTCAA | 1140 | |
| | TCTGGCGCCG CTGTGGTGCA CGAAATCGTG CGGTCTTTTG GCACCCTGAA GAAAGAAGGA | 1200 | |
| | TGGCGCCCCA GACGGACCAT CCTGTTTGCC TCTTGGGACG CCGAGGAATT TGGCCTGCTG | 1260 | |
| | GGATCTACAG AGTGGGCCGA AGAGAACAGC AGACTGCTGC AAGAAAGAGG CGTGGCCTAC | 1320 | |
| | ATCAATGCCG ACAGCAGCAT CGAGGGCAAC TACACCCTGA GAGTGGACTG CACCCCTCTG | 1380 | |
| | ATGTACTCCC TGGTGCACAA CCTGACCAAA GAGCTGAAGT CCCCTGACGA GGGCTTTGAG | 1440 | |
| | GGCAAGAGCC TGTACGAGTC CTGGACCAAG AAGTCCCCAT CTCCTGAGTT CAGCGGCATG | 1500 | |
| | CCCAGAATCA GCAAGCTCGG CTCCGGCAAT GACTTCGAGG TGTTCTTCCA GCGGCTGGGA | 1560 | |
| | ATCGCTTCTG GCAGAGCCAG ATACACCAAG AACTGGGAGA CAAACAAGTT CTCCGGCTAT | 1620 | |
| | CCCCTGTACC ACAGCGTGTA CGAGACATAC GAGCTGGTGG AAAAGTTCTA CGACCCCATG | 1680 | |
| | TTCAAGTACC ACCTGACAGT GGCCCAAGTG CGCGGAGGCA TGGTGTTCGA ACTGGCCAAT | 1740 | |
| | AGCATCGTGC TGCCCTTCGA CTGCAGAGAC TATGCCGTGG TGCTGCGGAA GTACGCCGAT | 1800 | |
| | AAGATCTACA GCATCTCCAT GAAGCACCCG CAAGAGATGA AGACCTACAG CGTGTCCTTC | 1860 | |
| | GACTCCCTGT TCTCTGCCGT GAAGAACTTC ACCGAGATCG CCAGCAAGTT CAGCGAGCGG | 1920 | |
| | CTGCAGGACT TCGACAAGAG CAACCCTATC GTGCTGAGGA TGATGAACGA CCAGCTGATG | 1980 | |
| | TTCCTGGAAC GCGCCTTCAT CGACCCACTG GGACTGCCCG ATAGACCCTT CTACCGGCAC | 2040 | |
| | GTGATCTATG CCCCTAGCAG CCACAACAAA TACGCCGGCG AGAGCTTCCC CGGCATCTAC | 2100 | |
| | GATGCCCTGT TCGACATCGA GAGCAAGGTG GACCCAAGCA AGGCCTGGGG AGAAGTGAAG | 2160 | |
| | CGGCAGATCT ACGTGGCCGC ATTCACAGTG CAGGCCGCTG CCGAAACACT GTCTGAGGTG | 2220 | |
| | GCCTCTGGCA GTGGTGAAGG CCGCGGATCT CTCCTGACTT GTGGGGATGT CGAAGAAAAC | 2280 | |
| | CCGGGACCCA TGGGAAGGGC AATGGTTGCG CGACTCGGCT TGGGCTTGCT TTTGCTGGCT | 2340 | |
| | CTTCTCCTCC CAACACAAAT TTACTCATCC GAAACGACCA CGGGAACTTC ATCTAATTCT | 2400 | |
| | AGTCAGTCAA CATCAAATTC CGGGCTTGCA CCCAATCCTA CGAACGCTAC CACGAAGGCG | 2460 | |
| | GCGGGAGGCG CTCTCCAATC CACCGCCTCA CTTTTCGTGG TTTCTCTCTC ACTTCTTCAT | 2520 | |
| | CTCTATTCT | 2529 | |
| B2M | GCATATAAAA CCTCAGCAGA AATAAAGAGG TTTTGTTGTT TGGTAAGAAC ATACCTTGGG | 60 | 81 |
| targeting left | TTGGTTGGGC ACGGTGGCTC GTGCCTGTAA TCCCAACACT TTGGGAGGCC AAGGCAGGCT | 120 | |
| homology | GATCACTTGA AGTTGGGAGT TCAAGACCAG CCTGGCCAAC ATGGTGAAAT CCCGTCTCTA | 180 | |
| arm (nucleic | CTGAAAATAC AAAAATTAAC CAGGCATGGT GGTGTGTGCC TGTAGTCCCA GGAATCACTT | 240 | |
| acid) | GAACCCAGGA GGCGGAGGTT GCAGTGAGCT GAGATCTCAC CACTGCACAC TGCACTCCAG | 300 | |
| | CCTGGGCAAT GGAATGAGAT TCCATCCCAA AAAATAAAAA AATAAAAAAA TAAAGAACAT | 360 | |
| | ACCTTGGGTT GATCCACTTA GGAACCTCAG ATAATAACAT CTGCCACGTA TAGAGCAATT | 420 | |
| | GCTATGTCCC AGGCACTCTA CTAGACACTT CATACAGTTT AGAAAATCAG ATGGGTGTAG | 480 | |
| | ATCAAGGCAG GAGCAGGAAC CAAAAAGAAA GGCATAAACA TAAGAAAAAA AATGGAAGGG | 540 | |
| | GTGGAAACAG AGTACAATAA CATGAGTAAT TTGATGGGGG CTATTATGAA CTGAGAAATG | 600 | |
| | AACTTTGAAA AGTATCTTGG GGCCAAATCA TGTAGACTCT TGAGTGATGT GTTAAGGAAT | 660 | |
| | GCTATGAGTG CTGAGAGGGC ATCAGAAGTC CTTGAGAGCC TCCAGAGAAA GGCTCTTAAA | 720 | |
| | AATGCAGCGC AATCTCCAGT GACAGAAGAT ACTGCTAGAA ATCTGCTAGA AAAAAAACAA | 780 | |
| | AAAAGGCATG TATAGAGGAA TTATGAGGGA AAGATACCAA GTCACGGTTT ATTCTTCAAA | 840 | |
| | ATGGAGGTGG CTTGTTGGGA AGGTGGAAGC TCATTTGGCC AGAGTGGAAA TGGAATTGGG | 900 | |
| | AGAAATCGAT GACCAAATGT AAACACTTGG TGCCTGATAT AGGTTGACAC CAAGTTAGCC | 960 | |
| | CCAAGTGAAA TACCCTGGCA ATATTAATGT GTCTTTTCCC GATATTCCTC AGGTACTCCA | 1020 | |
| | AAGATTCAGG TTTACTCACG TC | 1042 | |
| B2M | ATCCAGCAGA GAATGGAAAG TCAAATTTCC TGAATTGCTA TGTGTCTGGG TTTCATCCAT | 60 | 82 |
| targeting | CCGACATTGA AGTTGACTTA CTGAAGAATG GAGAGAGAAT TGAAAAAGTG GAGCATTCAG | 120 | |
| right | ACTTGTCTTT CAGCAAGGAC TGGTCTTTCT ATCTCTTGTA CTACACTGAA TTCACCCCCA | 180 | |
| homology | CTGAAAAAGA TGAGTATGCC TGCCGTGTGA ACCATGTGAC TTTGTCACAG CCCAAGATAG | 240 | |
| arm (nucleic | TTAAGTGGGG TAAGTCTTAC ATTCTTTTGT AAGCTGCTGA AAGTTGTGTA TGAGTAGTCA | 300 | |
| acid) | TATCATAAAG CTGCTTTGAT ATAAAAAAGG TCTATGGCCA TACTACCCTG AATGAGTCCC | 360 | |
| | ATCCCATCTG ATATAAACAA TCTGCATATT GGGATTGTCA GGGAATGTTC TTAAAGATCA | 420 | |
| | GATTAGTGGC ACCTGCTGAG ATACTGATGC ACAGCATGGT TTCTGAACCA GTAGTTTCCC | 480 | |
| | TGCAGTTGAG CAGGGAGCAG CAGCAGCACT TGCACAAATA CATATACACT CTTAACACTT | 540 | |
| | CTTACCTACT GGCTTCCTCT AGCTTTTGTG GCAGCTTCAG GTATATTTAG CACTGAACGA | 600 | |
| | ACATCTCAAG AAGGTATAGG CCTTTGTTTG TAAGTCCTGC TGTCCTAGCA TCCTATAATC | 660 | |
| | CTGGACTTCT CCAGTACTTT CTGGCTGGAT TGGTATCTGA GGCTAGTAGG AAGGGCTTGT | 720 | |
| | TCCTGCTGGG TAGCTCTAAA CAATGTATTC ATGGGTAGGA ACAGCAGCCT ATTCTGCCAG | 780 | |
| | CCTTATTTCT AACCATTTTA GACATTTGTT AGTACATGGT ATTTTAAAAG TAAAACTTAA | 840 | |
| | TGTCTTCCTT TTTTTTCTCC ACTGTCTTTT TCATAGATCG AGACATGTAA GCAGCATCAT | 900 | |
| | GGAGGTAAGT TTTTGACCTT GAGAAAATGT TTTTGTTTCA CTGTCCTGAG GACTATTTAT | 960 | |
| | AGACAGCTCT AACATGATAA CCCTCACTAT GTGGAGAACA TTGACAGAGT AACATTTTAG | 1020 | |
| | GAG | 1023 | |
| B2M | GCATATAAAA CCTCAGCAGA AATAAAGAGG TTTTGTTGTT TGGTAAGAAC ATACCTTGGG | 60 | 83 |
| targeting | TTGGTTGGGC ACGGTGGCTC GTGCCTGTAA TCCCAACACT TTGGGAGGCC AAGGCAGGCT | 120 | |
| plasmid | GATCACTTGA AGTTGGGAGT TCAAGACCAG CCTGGCCAAC ATGGTGAAAT CCCGTCTCTA | 180 | |
| (nucleic acid) | CTGAAAATAC AAAAATTAAC CAGGCATGGT GGTGTGTGCC TGTAGTCCCA GGAATCACTT | 240 | |
| | GAACCCAGGA GGCGGAGGTT GCAGTGAGCT GAGATCTCAC CACTGCACAC TGCACTCCAG | 300 | |
| | CCTGGGCAAT GGAATGAGAT TCCATCCCAA AAAATAAAAA AATAAAAAAA TAAAGAACAT | 360 | |
| | ACCTTGGGTT GATCCACTTA GGAACCTCAG ATAATAACAT CTGCCACGTA TAGAGCAATT | 420 | |
| | GCTATGTCCC AGGCACTCTA CTAGACACTT CATACAGTTT AGAAAATCAG ATGGGTGTAG | 480 | |
| | ATCAAGGCAG GAGCAGGAAC CAAAAAGAAA GGCATAAACA TAAGAAAAAA AATGGAAGGG | 540 | |
| | GTGGAAACAG AGTACAATAA CATGAGTAAT TTGATGGGGG CTATTATGAA CTGAGAAATG | 600 | |
| | AACTTTGAAA AGTATCTTGG GGCCAAATCA TGTAGACTCT TGAGTGATGT GTTAAGGAAT | 660 | |
| | GCTATGAGTG CTGAGAGGGC ATCAGAAGTC CTTGAGAGCC TCCAGAGAAA GGCTCTTAAA | 720 | |
| | AATGCAGCGC AATCTCCAGT GACAGAAGAT ACTGCTAGAA ATCTGCTAGA AAAAAAACAA | 780 | |
| | AAAAGGCATG TATAGAGGAA TTATGAGGGA AAGATACCAA GTCACGGTTT ATTCTTCAAA | 840 | |
| | ATGGAGGTGG CTTGTTGGGA AGGTGGAAGC TCATTTGGCC AGAGTGGAAA TGGAATTGGG | 900 | |
| | AGAAATCGAT GACCAAATGT AAACACTTGG TGCCTGATAT AGGTTGACAC CAAGTTAGCC | 960 | |
| | CCAAGTGAAA TACCCTGGCA ATATTAATGT GTCTTTTCCC GATATTCCTC AGGTACTCCA | 1020 | |
| | AAGATTCAGG TTTACTCACG TCGGCCTCCA ACGCGTAGAT CTATTGATTA TTGACTAGTT | 1080 | |
| | ATTAATAGTA ATCAATTACG GGGTCATTAG TTCATAGCCC ATATATGGAG TTCCGCGTTA | 1140 | |
| | CATAACTTAC GGTAAATGGC CCGCCTGGCT GACCGCCCAA CGACCCCCGC CCATTGACGT | 1200 | |
| | CAATAATGAC GTATGTTCCC ATAGTAACGC CAATAGGGAC TTTCCATTGA CGTCAATGGG | 1260 | |
| | TGGACTATTT ACGGTAAACT GCCCACTTGG CAGTACATCA AGTGTATCAT ATGCCAAGTA | 1320 | |
| | CGCCCCCTAT TGACGTCAAT GACGGTAAAT GGCCCGCCTG GCATTATGCC CAGTACATGA | 1380 | |
| | CCTTATGGGA CTTTCCTACT TGGCAGTACA TCTACGTATT AGTCATCGCT ATTACCATGG | 1440 | |
| | GTCGAGGTGA GCCCCACGTT CTGCTTCACT CTCCCCATCT CCCCCCCCTC CCCACCCCCA | 1500 | |
| | ATTTTGTATT TATTTATTTT TTAATTATTT TGTGCAGCGA TGGGGGCGGG GGGGGGGGGG | 1560 | |
| | GCGCGCGCCA GGGGGGGGGG GGCGGGGCGA GGGGGGGGGG GGGGCGAGGC GGAGAGGTGC | 1620 | |
| | GGCGGCAGCC AATCAGAGCG GCGCGCTCCG AAAGTTTCCT TTTATGGCGA GGCGGCGGCG | 1680 | |
| | GCGGCGGCCC TATAAAAAGC GAAGCGCGCG GCGGGCGGGA GTCGCTGCGT TGCCTTCGCC | 1740 | |
| | CCGTGCCCCG CTCCGCGCCG CCTCGCGCCG CCCGCCCCGG CTCTGACTGA CCGCGTTACT | 1800 | |
| | CCCACAGGTG AGCGGGCGGG ACGGCCCTTC TCCTCCGGGC TGTAATTAGC GCTTGGTTTA | 1860 | |
| | ATGACGGCTC GTTTCTTTTC TGTGGCTGCG TGAAAGCCTT AAAGGGCTCC GGGAGGGCCC | 1920 | |
| | TTTGTGCGGG GGGGAGGGGC TCGGGGGGTG CGTGCGTGTG TGTGTGCGTG GGGAGCGCCG | 1980 | |
| | CGTGCGGCCC GCGCTGCCCG GCGGCTGTGA GCGCTGCGGG CGCGGCGCGG GGCTTTGTGC | 2040 | |
| | GCTCCGCGTG TGCGCGAGGG GAGCGCGGCC GGGGGCGGTG CCCCGCGGTG CGGGGGGGCT | 2100 | |
| | GCGAGGGGAA CAAAGGCTGC GTGCGGGGTG TGTGCGTGGG GGGGTGAGCA GGGGGTGTGG | 2160 | |
| | GCGCGGCGGT CGGGCTGTAA CCCCCCCCTG CACCCCCCTC CCCGAGTTGC TGAGCACGGC | 2220 | |
| | CCGGCTTCGG GTGCGGGGCT CCGTGCGGGG CGTGGCGCGG GGCTCGCCGT GCCGGGCGGG | 2280 | |
| | GGGTGGCGGC AGGTGGGGGT GCCGGGCGGG GCGGGGCCGC CTCGGGCCGG GGAGGGCTCG | 2340 | |
| | GGGGAGGGGC GCGGCGGCCC CGGAGCGCCG GCGGCTGTCG AGGCGCGGCG AGCCGCAGCC | 2400 | |
| | ATTGCCTTTT ATGGTAATCG TGCGAGAGGG CGCAGGGACT TCCTTTGTCC CAAATCTGGC | 2460 | |
| | GGAGCCGAAA TCTGGGAGGC GCCGCCGCAC CCCCTCTAGC GGGCGCGGGC GAAGCGGTGC | 2520 | |
| | GGCGCCGGCA GGAAGGAAAT GGGCGGGGAG GGCCTTCGTG CGTCGCCGCG CCGCCGTCCC | 2580 | |
| | CTTCTCCATC TCCAGCCTCG GGGCTGCCGC AGGGGGACGG CTGCCTTCGG GGGGGACGGG | 2640 | |
| | GCAGGGCGGG GTTCGGCTTC TGGCGTGTGA CCGGCGGGAT ATCTACGAAG CGGCCGCCCT | 2700 | |
| | CTGCTAACCA TGTTCATGCC TTCTTCTTTT TCCTACAGCT CCTGGGCAAC GTGCTGGTTA | 2760 | |
| | TTGTGCTGTC TCATCATTTT GGCAAAGTCG ACGCCACCAT GGTGGTCATG GCCCCTAGAA | 2820 | |
| | CACTGTTCCT GCTGCTGTCT GGCGCCCTGA CACTGACAGA GACATGGGCC GTGATGGCCC | 2880 | |
| | CCAGAACCCT GATCCTGGGC GGCGGTGGTT CAGGCGGAGG AGGTTCAGGA GGAGGGGGTA | 2940 | |
| | GTGGAGGTGG TGGTTCTATC CAGCGGACCC CTAAGATCCA GGTGTACAGC AGACACCCCG | 3000 | |
| | CCGAGAACGG CAAGAGCAAC TTCCTGAACT GCTACGTGTC CGGCTTTCAC CCCAGCGACA | 3060 | |
| | TTGAGGTGGA CCTGCTGAAG AACGGCGAGC GGATCGAGAA GGTGGAACAC AGCGATCTGA | 3120 | |
| | GCTTCAGCAA GGACTGGTCC TTCTACCTGC TGTACTACAC CGAGTTCACC CCTACCGAGA | 3180 | |
| | AGGACGAGTA CGCCTGCAGA GTGAACCACG TGACACTGAG CCAGCCTAAG ATCGTGAAGT | 3240 | |
| | GGGATCGCGA TATGGGCGGA GGCGGATCTG GTGGCGGAGG AAGTGGCGGC GGAGGATCTG | 3300 | |
| | GCTCCCACTC CTTGAAGTAT TTCCACACTT CCGTGTCCCG GCCCGGCCGC GGGGAGCCCC | 3360 | |
| | GCTTCATCTC TGTGGGCTAC GTGGACGACA CCCAGTTCGT GCGCTTCGAC AACGACGCCG | 3420 | |
| | CGAGTCCGAG GATGGTGCCG CGGGCGCCGT GGATGGAGCA GGAGGGGTCA GAGTATTGGG | 3480 | |
| | ACCGGGAGAC ACGGAGCGCC AGGGACACCG CACAGATTTT CCGAGTGAAT CTGCGGACGC | 3540 | |
| | TGCGCGGCTA CTACAATCAG AGCGAGGCCG GGTCTCACAC CCTGCAGTGG ATGCATGGCT | 3600 | |
| | GCGAGCTGGG GCCCGACGGG CGCTTCCTCC GCGGGTATGA ACAGTTCGCC TACGACGGCA | 3660 | |
| | AGGATTATCT CACCCTGAAT GAGGACCTGC GCTCCTGGAC CGCGGTGGAC ACGGCGGCTC | 3720 | |
| | AGATCTCCGA GCAAAAGTCA AATGATGCCT CTGAGGCGGA GCACCAGAGA GCCTACCTGG | 3780 | |
| | AAGACACATG CGTGGAGTGG CTCCACAAAT ACCTGGAGAA GGGGAAGGAG ACGCTGCTTC | 3840 | |
| | ACCTGGAGCC CCCAAAGACA CACGTGACTC ACCACCCCAT CTCTGACCAT GAGGCCACCC | 3900 | |
| | TGAGGTGCTG GGCCCTGGGC TTCTACCCTG CGGAGATCAC ACTGACCTGG CAGCAGGATG | 3960 | |
| | GGGAGGGCCA TACCCAGGAC ACGGAGCTCG TGGAGACCAG GCCTGCAGGG GATGGAACCT | 4020 | |
| | TCCAGAAGTG GGCAGCTGTG GTGGTGCCTT CTGGAGAGGA GCAGAGATAC ACGTGCCATG | 4080 | |
| | TGCAGCATGA GGGGCTACCC GAGCCCGTCA CCCTGAGATG GAAGCCGGCT TCCCAGCCCA | 4140 | |
| | CCATCCCCAT CGTGGGCATC ATTGCTGGCC TGGTTCTCCT TGGATCTGTG GTCTCTGGAG | 4200 | |
| | CTGTGGTTGC TGCTGTGATA TGGAGGAAGA AGAGCTCAGG TGGAAAAGGA GGGAGCTACT | 4260 | |
| | CTAAGGCTGA GTGGAGCGAC AGTGCCCAGG GGTCTGAGTC TCACAGCTTG TAATGAAGCG | 4320 | |
| | GCCGCGACTC TAGATCATAA TCAGCCATAC CACATTTGTA GAGGTTTTAC TTGCTTTAAA | 4380 | |
| | AAACCTCCCA CACCTCCCCC TGAACCTGAA ACATAAAATG AATGCAATTG TTGTTGTTAA | 4440 | |
| | CTTGTTTATT GCAGCTTATA ATGGTTACAA ATAAAGCAAT AGCATCACAA ATTTCACAAA | 4500 | |
| | TAAAGCATTT TTTTCACTGC ATTCTAGTTG TGGTTTGTCC AAACTCATCA ATGTATCTTA | 4560 | |
| | TCATGTCTGA TCCAGCAGAG AATGGAAAGT CAAATTTCCT GAATTGCTAT GTGTCTGGGT | 4620 | |
| | TTCATCCATC CGACATTGAA GTTGACTTAC TGAAGAATGG AGAGAGAATT GAAAAAGTGG | 4680 | |
| | AGCATTCAGA CTTGTCTTTC AGCAAGGACT GGTCTTTCTA TCTCTTGTAC TACACTGAAT | 4740 | |
| | TCACCCCCAC TGAAAAAGAT GAGTATGCCT GCCGTGTGAA CCATGTGACT TTGTCACAGC | 4800 | |
| | CCAAGATAGT TAAGTGGGGT AAGTCTTACA TTCTTTTGTA AGCTGCTGAA AGTTGTGTAT | 4860 | |
| | GAGTAGTCAT ATCATAAAGC TGCTTTGATA TAAAAAAGGT CTATGGCCAT ACTACCCTGA | 4920 | |
| | ATGAGTCCCA TCCCATCTGA TATAAACAAT CTGCATATTG GGATTGTCAG GGAATGTTCT | 4980 | |
| | TAAAGATCAG ATTAGTGGCA CCTGCTGAGA TACTGATGCA CAGCATGGTT TCTGAACCAG | 5040 | |
| | TAGTTTCCCT GCAGTTGAGC AGGGAGCAGC AGCAGCACTT GCACAAATAC ATATACACTC | 5100 | |
| | TTAACACTTC TTACCTACTG GCTTCCTCTA GCTTTTGTGG CAGCTTCAGG TATATTTAGC | 5160 | |
| | ACTGAACGAA CATCTCAAGA AGGTATAGGC CTTTGTTTGT AAGTCCTGCT GTCCTAGCAT | 5220 | |
| | CCTATAATCC TGGACTTCTC CAGTACTTTC TGGCTGGATT GGTATCTGAG GCTAGTAGGA | 5280 | |
| | AGGGCTTGTT CCTGCTGGGT AGCTCTAAAC AATGTATTCA TGGGTAGGAA CAGCAGCCTA | 5340 | |
| | TTCTGCCAGC CTTATTTCTA ACCATTTTAG ACATTTGTTA GTACATGGTA TTTTAAAAGT | 5400 | |
| | AAAACTTAAT GTCTTCCTTT TTTTTCTCCA CTGTCTTTTT CATAGATCGA GACATGTAAG | 5460 | |
| | CAGCATCATG GAGGTAAGTT TTTGACCTTG AGAAAATGTT TTTGTTTCAC TGTCCTGAGG | 5520 | |
| | ACTATTTATA GACAGCTCTA ACATGATAAC CCTCACTATG TGGAGAACAT TGACAGAGTA | 5580 | |
| | ACATTTTAGC AGAGGCTAGG TGGAGGCTCA GTGATGATAA GTCTGCGATG GTGGATGCAT | 5640 | |
| | GTGTCATGGT CATAGCTGTT TCCTGTGTGA AATTGTTATC CGCTCAGAGG GCACAATCCT | 5700 | |
| | ATTCCGCGCT ATCCGACAAT CTCCAAGACA TTAGGTGGAG TTCAGTTCGG CGTATGGCAT | 5760 | |
| | ATGTCGCTGG AAAGAACATG TGAGCAAAAG GCCAGCAAAA GGCCAGGAAC CGTAAAAAGG | 5820 | |
| | CCGCGTTGCT GGCGTTTTTC CATAGGCTCC GCCCCCCTGA CGAGCATCAC AAAAATCGAC | 5880 | |
| | GCTCAAGTCA GAGGTGGCGA AACCCGACAG GACTATAAAG ATACCAGGCG TTTCCCCCTG | 5940 | |
| | GAAGCTCCCT CGTGCGCTCT CCTGTTCCGA CCCTGCCGCT TACCGGATAC CTGTCCGCCT | 6000 | |
| | TTCTCCCTTC GGGAAGCGTG GCGCTTTCTC ATAGCTCACG CTGTAGGTAT CTCAGTTCGG | 6060 | |
| | TGTAGGTCGT TCGCTCCAAG CTGGGCTGTG TGCACGAACC CCCCGTTCAG CCCGACCGCT | 6120 | |
| | GCGCCTTATC CGGTAACTAT CGTCTTGAGT CCAACCCGGT AAGACACGAC TTATCGCCAC | 6180 | |
| | TGGCAGCAGC CACTGGTAAC AGGATTAGCA GAGCGAGGTA TGTAGGCGGT GCTACAGAGT | 6240 | |
| | TCTTGAAGTG GTGGCCTAAC TACGGCTACA CTAGAAGAAC AGTATTTGGT ATCTGCGCTC | 6300 | |
| | TGCTGAAGCC AGTTACCTTC GGAAAAAGAG TTGGTAGCTC TTGATCCGGC AAACAAACCA | 6360 | |
| | CCGCTGGTAG CGGTGGTTTT TTTGTTTGCA AGCAGCAGAT TACGCGCAGA AAAAAAGGAT | 6420 | |
| | CTCAAGAAGA TCCTTTGATC TTTTCTACGG GGTCTGACGC TCTATTCAAC AAAGCCGCCG | 6480 | |
| | TCCCGTCAAG TCAGCGTAAA TGGGTAGGGG GCTTCAAATC GTCCTCGTGA TACCAATTCG | 6540 | |
| | GAGCCTGCTT TTTTGTACAA ACTTGTTGAT AATGGCAATT CAAGGATCTT CACCTAGATC | 6600 | |
| | CTTTTAAATT AAAAATGAAG TTTTAAATCA ATCTAAAGTA TATATGAGTA AACTTGGTCT | 6660 | |
| | GACAGTTACC AATGCTTAAT CAGTGAGGCA CCTATCTCAG CGATCTGTCT ATTTCGTTCA | 6720 | |
| | TCCATAGTTG CCTGACTCCC CGTCGTGTAG ATAACTACGA TACGGGAGGG CTTACCATCT | 6780 | |
| | GGCCCCAGTG CTGCAATGAT ACCGCGAGAG CCACGCTCAC CGGCTCCAGA TTTATCAGCA | 6840 | |
| | ATAAACCAGC CAGCCGGAAG GGCCGAGCGC AGAAGTGGTC CTGCAACTTT ATCCGCCTCC | 6900 | |
| | ATCCAGTCTA TTAATTGTTG CCGGGAAGCT AGAGTAAGTA GTTCGCCAGT TAATAGTTTG | 6960 | |
| | CGCAACGTTG TTGCCATTGC TACAGGCATC GTGGTGTCAC GCTCGTCGTT TGGTATGGCT | 7020 | |
| | TCATTCAGCT CCGGTTCCCA ACGATCAAGG CGAGTTACAT GATCCCCCAT GTTGTGCAAA | 7080 | |
| | AAAGCGGTTA GCTCCTTCGG TCCTCCGATC GTTGTCAGAA GTAAGTTGGC CGCAGTGTTA | 7140 | |
| | TCACTCATGG TTATGGCAGC ACTGCATAAT TCTCTTACTG TCATGCCATC CGTAAGATGC | 7200 | |
| | TTTTCTGTGA CTGGTGAGTA CTCAACCAAG TCATTCTGAG AATAGTGTAT GCGGCGACCG | 7260 | |
| | AGTTGCTCTT GCCCGGCGTC AATACGGGAT AATACCGCGC CACATAGCAG AACTTTAAAA | 7320 | |
| | GTGCTCATCA TTGGAAAACG TTCTTCGGGG CGAAAACTCT CAAGGATCTT ACCGCTGTTG | 7380 | |
| | AGATCCAGTT CGATGTAACC CACTCGTGCA CCCAACTGAT CTTCAGCATC TTTTACTTTC | 7440 | |
| | ACCAGCGTTT CTGGGTGAGC AAAAACAGGA AGGCAAAATG CCGCAAAAAA GGGAATAAGG | 7500 | |
| | GCGACACGGA AATGTTGAAT ACTCATACTC TTCCTTTTTC AATATTATTG AAGCATTTAT | 7560 | |
| | CAGGGTTATT GTCTCATGAG CGGATACATA TTTGAATGTA TTTAGAAAAA TAAACAAATA | 7620 | |
| | GGGGTTCCGC GCACATTTCC CCGAAAAGTG CCAGATACCT GAAACAAAAC CCATCGTACG | 7680 | |
| | GCCAAGGAAG TCTCCAATAA CTGTGATCCA CCACAAGCGC CAGGGTTTTC CCAGTCACGA | 7740 | |
| | CGTTGTAAAA CGACGGCCAG TCATGCATAA TCCGCACGCA TCTGGAATAA GGAAGTGCCA | 7800 | |
| | TTCCGCCTGA CCT | 7813 | |
| AAVS1 | ACTCTGCCCC AGGCCTCCTT ACCATTCCCC TTCGACCTAC TCTCTTCCGC ATTGGAGTCG | 60 | 84 |
| targeting left | CTTTAACTGG CCCTGGCTTT GGCAGCCTGT GCTGACCCAT GCAGTCCTCC TTACCATCCC | 120 | |
| homology | TCCCTCGACT TCCCCTCTTC CGATGTTGAG CCCCTCCAGC CGGTCCTGGA CTTTGTCTCC | 180 | |
| arm (nucleic | TTCCCTGCCC TGCCCTCTCC TGAACCTGAG CCAGCTCCCA TAGCTCAGTC TGGTCTATCT | 240 | |
| acid) | GCCTGGCCCT GGCCATTGTC ACTTTGCGCT GCCCTCCTCT CGCCCCCGAG TGCCCTTGCT | 300 | |
| | GTGCCGCCGG AACTCTGCCC TCTAACGCTG CCGTCTCTCT CCTGAGTCCG GACCACTTTG | 360 | |
| | AGCTCTACTG GCTTCTGCGC CGCCTCTGGC CCACTGTTTC CCCTTCCCAG GCAGGTCCTG | 420 | |
| | CTTTCTCTGA CCTGCATTCT CTCCCCTGGG CCTGTGCCGC TTTCTGTCTG CAGCTTGTGG | 480 | |
| | CCTGGGTCAC CTCTACGGCT GGCCCAGATC CTTCCCTGCC GCCTCCTTCA GGTTCCGTCT | 540 | |
| | TCCTCCACTC CCTCTTCCCC TTGCTCTCTG CTGTGTTGCT GCCCAAGGAT GCTCTTTCCG | 600 | |
| | GAGCACTTCC TTCTCGGCGC TGCACCACGT GATGTCCTCT GAGCGGATCC TCCCCGTGTC | 660 | |
| | TGGGTCCTCT CCGGGCATCT CTCCTCCCTC ACCCAACCCC ATGCCGTCTT CACTCGCTGG | 720 | |
| | GTTCCCTTTT CCTTCTCCTT CTGGGGCCTG TGCCATCTCT CGTTTCTTAG GATGGCCTTC | 780 | |
| | TCCGACGGAT GTCTCCCTTG CGTCCCGCCT CCCCTTCTTG TAGGCCTGCA TCATCACCGT | 840 | |
| | TTTTCTGGAC AACCCCAAAG TACCCCGTCT CCCTGGCTTT AGCCACCTCT CCATCCTCTT | 900 | |
| | GCTTTCTTTG CCTGGACACC CCGTTCTCCT GTGGATTCGG GTCACCTCTC ACTCCTTTCA | 960 | |
| | TTTGGGCAGC TCCCCTACCC CCCTTACCTC TCTAGTCTGT GCTAGCTCTT CCAGCCCCCT | 1020 | |
| | GTCATGGCAT CTTCCAGGGG TCCGAGAGCT CAGCTAGTCT TCTTCCTCCA ACCCGGGCCC | 1080 | |
| | CTATGTCCAC TTCAGGACAG CATGTTTGCT GCCTCCAGGG ATCCTGTGTC CCCGAGCTGG | 1140 | |
| | GACCACCTTA TATTCCCAGG GCCGGTTAAT GTGGCTCTGG TTCTGGGTAC TTTTATCTGT | 1200 | |
| | CCCCT | 1205 | |
| AAVS1 | CCACCCCACA GTGGGGCCAC TAGGGACAGG ATTGGTGACA GAAAAGCCCC ATCCTTAGGC | 60 | 85 |
| targeting | CTCCTCCTTC CTAGTCTCCT GATATTGGGT CTAACCCCCA CCTCCTGTTA GGCAGATTCC | 120 | |
| right | TTATCTGGTG ACACACCCCC ATTTCCTGGA GCCATCTCTC TCCTTGCCAG AACCTCTAAG | 180 | |
| homology | GTTTGCTTAC GATGGAGCCA GAGAGGATCC TGGGAGGGAG AGCTTGGCAG GGGGTGGGAG | 240 | |
| arm (nucleic | GGAAGGGGGG GATGCGTGAC CTGCCCGGTT CTCAGTGGCC ACCCTGCGCT ACCCTCTCCC | 300 | |
| acid) | AGAACCTGAG CTGCTCTGAC GCGGCCGTCT GGTGCGTTTC ACTGATCCTG GTGCTGCAGC | 360 | |
| | TTCCTTACAC TTCCCAAGAG GAGAAGCAGT TTGGAAAAAC AAAATCAGAA TAAGTTGGTC | 420 | |
| | CTGAGTTCTA ACTTTGGCTC TTCACCTTTC TAGTCCCCAA TTTATATTGT TCCTCCGTGC | 480 | |
| | GTCAGTTTTA CCTGTGAGAT AAGGCCAGTA GCCAGCCCCG TCCTGGCAGG GCTGTGGTGA | 540 | |
| | GGAGGGGGGT GTCCGTGTGG AAAACTCCCT TTGTGAGAAT GGTGCGTCCT AGGTGTTCAC | 600 | |
| | CAGGTCGTGG CCGCCTCTAC TCCCTTTCTC TTTCTCCATC CTTCTTTCCT TAAAGAGTCC | 660 | |
| | CCAGTGCTAT CTGGGACATA TTCCTCCGCC CAGAGCAGGG TCCCGCTTCC CTAAGGCCCT | 720 | |
| | GCTCTGGGCT TCTGGGTTTG AGTCCTTGGC AAGCCCAGGA GAGGCGCTCA GGCTTCCCTG | 780 | |
| | TCCCGCTTCC TCGTCCACCA TCTCATGCCC CTGGCTCTCC TGCCCCTTCC CTACAGGGGT | 840 | |
| | TCCTGGCTCT GCTCTTCAGA CTGAGCCCCG TTCCCCTGCA TCCCCGTACC CCTGCATCCC | 900 | |
| | CCTTCCCCTG CATCCCCCAG AGGCCCCAGG CCACCTACTT GGCCTGGACC CCACGAGAGG | 960 | |
| | CCACCCCAGC CCTGTCTACC AGGCTGCCTT TTGGGTGGAT TCTCCTCCAA CTGTGGGGTG | 1020 | |
| | ACTGCTTGGC AAACTCACTC TTCGGGGTAT CCCAGGAGGC CTGGAGCATT GGGGTGGGCT | 1080 | |
| | GGGGTTCAGA GAGGAGGGAT TCCCTTCTCA GGTTACGTGG CCAAGAAGCA GGGGAGCTGG | 1140 | |
| | GTTTGGGTCA GGTCTGGGTG TGGGGTGACC AGCTTATGCT GTTTGCCCAG GACAGCCTAG | 1200 | |
| AAVS1 | ACTCTGCCCC AGGCCTCCTT ACCATTCCCC TTCGACCTAC TCTCTTCCGC ATTGGAGTCG | 60 | 86 |
| targeting | CTTTAACTGG CCCTGGCTTT GGCAGCCTGT GCTGACCCAT GCAGTCCTCC TTACCATCCC | 120 | |
| plasmid | TCCCTCGACT TCCCCTCTTC CGATGTTGAG CCCCTCCAGC CGGTCCTGGA CTTTGTCTCC | 180 | |
| (nucleic acid) | TTCCCTGCCC TGCCCTCTCC TGAACCTGAG CCAGCTCCCA TAGCTCAGTC TGGTCTATCT | 240 | |
| | GCCTGGCCCT GGCCATTGTC ACTTTGCGCT GCCCTCCTCT CGCCCCCGAG TGCCCTTGCT | 300 | |
| | GTGCCGCCGG AACTCTGCCC TCTAACGCTG CCGTCTCTCT CCTGAGTCCG GACCACTTTG | 360 | |
| | AGCTCTACTG GCTTCTGCGC CGCCTCTGGC CCACTGTTTC CCCTTCCCAG GCAGGTCCTG | 420 | |
| | CTTTCTCTGA CCTGCATTCT CTCCCCTGGG CCTGTGCCGC TTTCTGTCTG CAGCTTGTGG | 480 | |
| | CCTGGGTCAC CTCTACGGCT GGCCCAGATC CTTCCCTGCC GCCTCCTTCA GGTTCCGTCT | 540 | |
| | TCCTCCACTC CCTCTTCCCC TTGCTCTCTG CTGTGTTGCT GCCCAAGGAT GCTCTTTCCG | 600 | |
| | GAGCACTTCC TTCTCGGCGC TGCACCACGT GATGTCCTCT GAGCGGATCC TCCCCGTGTC | 660 | |
| | TGGGTCCTCT CCGGGCATCT CTCCTCCCTC ACCCAACCCC ATGCCGTCTT CACTCGCTGG | 720 | |
| | GTTCCCTTTT CCTTCTCCTT CTGGGGCCTG TGCCATCTCT CGTTTCTTAG GATGGCCTTC | 780 | |
| | TCCGACGGAT GTCTCCCTTG CGTCCCGCCT CCCCTTCTTG TAGGCCTGCA TCATCACCGT | 840 | |
| | TTTTCTGGAC AACCCCAAAG TACCCCGTCT CCCTGGCTTT AGCCACCTCT CCATCCTCTT | 900 | |
| | GCTTTCTTTG CCTGGACACC CCGTTCTCCT GTGGATTCGG GTCACCTCTC ACTCCTTTCA | 960 | |
| | TTTGGGCAGC TCCCCTACCC CCCTTACCTC TCTAGTCTGT GCTAGCTCTT CCAGCCCCCT | 1020 | |
| | GTCATGGCAT CTTCCAGGGG TCCGAGAGCT CAGCTAGTCT TCTTCCTCCA ACCCGGGCCC | 1080 | |
| | CTATGTCCAC TTCAGGACAG CATGTTTGCT GCCTCCAGGG ATCCTGTGTC CCCGAGCTGG | 1140 | |
| | GACCACCTTA TATTCCCAGG GCCGGTTAAT GTGGCTCTGG TTCTGGGTAC TTTTATCTGT | 1200 | |
| | CCCCTGCGGC CGCACGCGTA GATCTATTGA TTATTGACTA GTTATTAATA GTAATCAATT | 1260 | |
| | ACGGGGTCAT TAGTTCATAG CCCATATATG GAGTTCCGCG TTACATAACT TACGGTAAAT | 1320 | |
| | GGCCCGCCTG GCTGACCGCC CAACGACCCC CGCCCATTGA CGTCAATAAT GACGTATGTT | 1380 | |
| | CCCATAGTAA CGCCAATAGG GACTTTCCAT TGACGTCAAT GGGTGGACTA TTTACGGTAA | 1440 | |
| | ACTGCCCACT TGGCAGTACA TCAAGTGTAT CATATGCCAA GTACGCCCCC TATTGACGTC | 1500 | |
| | AATGACGGTA AATGGCCCGC CTGGCATTAT GCCCAGTACA TGACCTTATG GGACTTTCCT | 1560 | |
| | ACTTGGCAGT ACATCTACGT ATTAGTCATC GCTATTACCA TGGGTCGAGG TGAGCCCCAC | 1620 | |
| | GTTCTGCTTC ACTCTCCCCA TCTCCCCCCC CTCCCCACCC CCAATTTTGT ATTTATTTAT | 1680 | |
| | TTTTTAATTA TTTTGTGCAG CGATGGGGGC GGGGGGGGGG GGGGCGCGCG CCAGGCGGGG | 1740 | |
| | CGGGGCGGGG CGAGGGGCGG GGCGGGGCGA GGCGGAGAGG TGCGGCGGCA GCCAATCAGA | 1800 | |
| | GCGGCGCGCT CCGAAAGTTT CCTTTTATGG CGAGGCGGCG GCGGCGGCGG CCCTATAAAA | 1860 | |
| | AGCGAAGCGC GCGGCGGGCG GGAGTCGCTG CGTTGCCTTC GCCCCGTGCC CCGCTCCGCG | 1920 | |
| | CCGCCTCGCG CCGCCCGCCC CGGCTCTGAC TGACCGCGTT ACTCCCACAG GTGAGCGGGC | 1980 | |
| | GGGACGGCCC TTCTCCTCCG GGCTGTAATT AGCGCTTGGT TTAATGACGG CTCGTTTCTT | 2040 | |
| | TTCTGTGGCT GCGTGAAAGC CTTAAAGGGC TCCGGGAGGG CCCTTTGTGC GGGGGGGAGC | 2100 | |
| | GGCTCGGGGG GTGCGTGCGT GTGTGTGTGC GTGGGGAGCG CCGCGTGCGG CCCGCGCTGC | 2160 | |
| | CCGGCGGCTG TGAGCGCTGC GGGCGCGGCG CGGGGCTTTG TGCGCTCCGC GTGTGCGCGA | 2220 | |
| | GGGGAGCGCG GCCGGGGGCG GTGCCCCGCG GTGCGGGGGG GCTGCGAGGG GAACAAAGGC | 2280 | |
| | TGCGTGCGGG GTGTGTGCGT GGGGGGGTGA GCAGGGGGTG TGGGCGCGGC GGTCGGGCTG | 2340 | |
| | TAACCCCCCC CTGCACCCCC CTCCCCGAGT TGCTGAGCAC GGCCCGGCTT CGGGTGCGGG | 2400 | |
| | GCTCCGTGCG GGGCGTGGCG CGGGGCTCGC GGTGCCGGGC GGGGGGTGGC GGCAGGTGGG | 2460 | |
| | GGTGCCGGGC GGGGCGGGGC CGCCTCGGGC CGGGGAGGGC TCGGGGGAGG GGCGCGGCGG | 2520 | |
| | CCCCGGAGCG CCGGCGGCTG TCGAGGCGCG GCGAGCCGCA GCCATTGCCT TTTATGGTAA | 2580 | |
| | TCGTGCGAGA GGGCGCAGGG ACTTCCTTTG TCCCAAATCT GGCGGAGCCG AAATCTGGGA | 2640 | |
| | GGCGCCGCCG CACCCCCTCT AGCGGGCGCG GGCGAAGCGG TGCGGCGCCG GCAGGAAGGA | 2700 | |
| | AATGGGCGGG GAGGGCCTTC GTGCGTCGCC GCGCCGCCGT CCCCTTCTCC ATCTCCAGCC | 2760 | |
| | TCGGGGCTGC CGCAGGGGGA CGGCTGCCTT CGGGGGGGAC GGGGCAGGGC GGGGTTCGGC | 2820 | |
| | TTCTGGCGTG TGACCGGCGG GATATCTACG AAGCGGCCGC CCTCTGCTAA CCATGTTCAT | 2880 | |
| | GCCTTCTTCT TTTTCCTACA GCTCCTGGGC AACGTGCTGG TTATTGTGCT GTCTCATCAT | 2940 | |
| | TTTGGCAAAG TCGACGCCAC CATGCTGCTG CTGGTCACAT CTCTGCTGCT GTGCGAGCTG | 3000 | |
| | CCCCATCCTG CCTTTCTGCT GATCCCCGAC ATCCAGATGA CCCAGACCAC AAGCAGCCTG | 3060 | |
| | TCTGCCAGCC TGGGCGATAG AGTGACCATC AGCTGTAGAG CCAGCCAGGA CATCAGCAAG | 3120 | |
| | TACCTGAACT GGTATCAGCA AAAGCCCGAC GGCACCGTGA AGCTGCTGAT CTACCACACC | 3180 | |
| | AGCAGACTGC ACAGCGGCGT GCCAAGCAGA TTTTCTGGCA GCGGCTCTGG CACCGACTAC | 3240 | |
| | AGCCTGACAA TCAGCAACCT GGAACAAGAG GATATCGCTA CCTACTTCTG CCAGCAAGGC | 3300 | |
| | AACACCCTGC CTTACACCTT TGGCGGAGGC ACCAAGCTGG AAATCACCGG CTCTACAAGC | 3360 | |
| | GGCAGCGGCA AACCTGGATC TGGCGAGGGA TCTACCAAGG GCGAAGTGAA ACTGCAAGAG | 3420 | |
| | TCTGGCCCTG GACTGGTGGC CCCATCTCAG TCTCTGAGCG TGACCTGTAC AGTCAGCGGA | 3480 | |
| | GTGTCCCTGC CTGATTACGG CGTGTCCTGG ATCAGACAGC CTCCTCGGAA AGGCCTGGAA | 3540 | |
| | TGGCTGGGAG TGATCTGGGG CAGCGAGACA ACCTACTACA ACAGCGCCCT GAAGTCCCGG | 3600 | |
| | CTGACCATCA TCAAGGACAA CTCCAAGAGC CAGGTGTTCC TGAAGATGAA CAGCCTGCAG | 3660 | |
| | ACCGACGACA CCGCCATCTA CTATTGCGCC AAGCACTACT ACTACGGCGG CAGCTACGCC | 3720 | |
| | ATGGATTATT GGGGCCAGGG CACCAGCGTG ACCGTGTCTA GCACGACGAC TCCTGCTCCA | 3780 | |
| | AGGCCTCCTA CACCTGCACC AACCATTGCA AGTCAGCCGT TGAGCCTCCG GCCAGAAGCA | 3840 | |
| | TGTCGCCCAG CCGCAGGCGG GGCTGTACAC ACGAGAGGCT TGGATTTCGC ATGTGACATC | 3900 | |
| | TATATCTGGG CCCCACTGGC CGGCACCTGC GGCGTGCTGC TGCTGAGCCT GGTGATCACC | 3960 | |
| | AAGCGAGGCC GCAAAAAACT CCTTTATATA TTCAAGCAAC CTTTTATGAG GCCCGTCCAG | 4020 | |
| | ACCACGCAAG AGGAAGATGG GTGCTCTTGC CGCTTTCCAG AGGAAGAGGA GGGGGGCTGC | 4080 | |
| | GAACTTAGAG TGAAGTTCAG CAGATCCGCC GATGCTCCCG CCTATCAGCA GGGCCAAAAC | 4140 | |
| | CAGCTGTACA ACGAGCTGAA CCTGGGGAGA AGAGAAGAGT ACGACGTGCT GGACAAGCGG | 4200 | |
| | AGAGGCAGAG ATCCTGAAAT GGGCGGCAAG CCCAGACGGA AGAATCCTCA AGAGGGCCTG | 4260 | |
| | TATAATGAGC TGCAGAAAGA CAAGATGGCC GAGGCCTACA GCGAGATCGG AATGAAGGGC | 4320 | |
| | GAGCGCAGAA GAGGCAAGGG ACACGATGGA CTGTACCAGG GCCTGAGCAC CGCCACCAAG | 4380 | |
| | GATACCTATG ATGCCCTGCA CATGCAGGCC CTGCCTCCAA GATAATAAAA CTTGTTTATT | 4440 | |
| | GCAGCTTATA ATGGTTACAA ATAAAGCAAT AGCATCACAA ATTTCACAAA TAAAGCATTT | 4500 | |
| | TTTTCACTGC ATTCTAGTTG TGGTTTGTCC AAACTCATCA ATGTATCTTA CCACCCCACA | 4560 | |
| | GTGGGGCCAC TAGGGACAGG ATTGGTGACA GAAAAGCCCC ATCCTTAGGC CTCCTCCTTC | 4620 | |
| | CTAGTCTCCT GATATTGGGT CTAACCCCCA CCTCCTGTTA GGCAGATTCC TTATCTGGTG | 4680 | |
| | ACACACCCCC ATTTCCTGGA GCCATCTCTC TCCTTGCCAG AACCTCTAAG GTTTGCTTAC | 4740 | |
| | GATGGAGCCA GAGAGGATCC TGGGAGGGAG AGCTTGGCAG GGGGTGGGAG GGAAGGGGGG | 4800 | |
| | GATGCGTGAC CTGCCCGGTT CTCAGTGGCC ACCCTGCGCT ACCCTCTCCC AGAACCTGAG | 4860 | |
| | CTGCTCTGAC GCGGCCGTCT GGTGCGTTTC ACTGATCCTG GTGCTGCAGC TTCCTTACAC | 4920 | |
| | TTCCCAAGAG GAGAAGCAGT TTGGAAAAAC AAAATCAGAA TAAGTTGGTC CTGAGTTCTA | 4980 | |
| | ACTTTGGCTC TTCACCTTTC TAGTCCCCAA TTTATATTGT TCCTCCGTGC GTCAGTTTTA | 5040 | |
| | CCTGTGAGAT AAGGCCAGTA GCCAGCCCCG TCCTGGCAGG GCTGTGGTGA GGAGGGGGGT | 5100 | |
| | GTCCGTGTGG AAAACTCCCT TTGTGAGAAT GGTGCGTCCT AGGTGTTCAC CAGGTCGTGG | 5160 | |
| | CCGCCTCTAC TCCCTTTCTC TTTCTCCATC CTTCTTTCCT TAAAGAGTCC CCAGTGCTAT | 5220 | |
| | CTGGGACATA TTCCTCCGCC CAGAGCAGGG TCCCGCTTCC CTAAGGCCCT GCTCTGGGCT | 5280 | |
| | TCTGGGTTTG AGTCCTTGGC AAGCCCAGGA GAGGCGCTCA GGCTTCCCTG TGCCCCTTCC | 5340 | |
| | TCGTCCACCA TCTCATGCCC CTGGCTCTCC TGCCCCTTCC CTACAGGGGT TCCTGGCTCT | 5400 | |
| | GCTCTTCAGA CTGAGCCCCG TTCCCCTGCA TCCCCGTACC CCTGCATCCC CCTTCCCCTG | 5460 | |
| | CATCCCCCAG AGGCCCCAGG CCACCTACTT GGCCTGGACC CCACGAGAGG CCACCCCAGC | 5520 | |
| | CCTGTCTACC AGGCTGCCTT TTGGGTGGAT TCTCCTCCAA CTGTGGGGTG ACTGCTTGGC | 5580 | |
| | AAACTCACTC TTCGGGGTAT CCCAGGAGGC CTGGAGCATT GGGGTGGGCT GGGGTTCAGA | 5640 | |
| | GAGGAGGGAT TCCCTTCTCA GGTTACGTGG CCAAGAAGCA GGGGAGCTGG GTTTGGGTCA | 5700 | |
| | GGTCTGGGTG TGGGGTGACC AGCTTATGCT GTTTGCCCAG GACAGCCTAG AGGCTAGGTG | 5760 | |
| | GAGGCTCAGT GATGATAAGT CTGCGATGGT GGATGCATGT GTCATGGTCA TAGCTGTTTC | 5820 | |
| | CTGTGTGAAA TTGTTATCCG CTCAGAGGGC ACAATCCTAT TCCGCGCTAT CCGACAATCT | 5880 | |
| | CCAAGACATT AGGTGGAGTT CAGTTCGGCG TATGGCATAT GTCGCTGGAA AGAACATGTG | 5940 | |
| | AGCAAAAGGC CAGCAAAAGG CCAGGAACCG TAAAAAGGCC GCGTTGCTGG CGTTTTTCCA | 6000 | |
| | TAGGCTCCGC CCCCCTGACG AGCATCACAA AAATCGACGC TCAAGTCAGA GGTGGCGAAA | 6060 | |
| | CCCGACAGGA CTATAAAGAT ACCAGGCGTT TCCCCCTGGA AGCTCCCTCG TGCGCTCTCC | 6120 | |
| | TGTTCCGACC CTGCCGCTTA CCGGATACCT GTCCGCCTTT CTCCCTTCGG GAAGCGTGGC | 6180 | |
| | GCTTTCTCAT AGCTCACGCT GTAGGTATCT CAGTTCGGTG TAGGTCGTTC GCTCCAAGCT | 6240 | |
| | GGGCTGTGTG CACGAACCCC CCGTTCAGCC CGACCGCTGC GCCTTATCCG GTAACTATCG | 6300 | |
| | TCTTGAGTCC AACCCGGTAA GACACGACTT ATCGCCACTG GCAGCAGCCA CTGGTAACAG | 6360 | |
| | GATTAGCAGA GCGAGGTATG TAGGCGGTGC TACAGAGTTC TTGAAGTGGT GGCCTAACTA | 6420 | |
| | CGGCTACACT AGAAGAACAG TATTTGGTAT CTGCGCTCTG CTGAAGCCAG TTACCTTCGG | 6480 | |
| | AAAAAGAGTT GGTAGCTCTT GATCCGGCAA ACAAACCACC GCTGGTAGCG GTGGTTTTTT | 6540 | |
| | TGTTTGCAAG CAGCAGATTA CGCGCAGAAA AAAAGGATCT CAAGAAGATC CTTTGATCTT | 6600 | |
| | TTCTACGGGG TCTGACGCTC TATTCAACAA AGCCGCCGTC CCGTCAAGTC AGCGTAAATG | 6660 | |
| | GGTAGGGGGC TTCAAATCGT CCTCGTGATA CCAATTCGGA GCCTGCTTTT TTGTACAAAC | 6720 | |
| | TTGTTGATAA TGGCAATTCA AGGATCTTCA CCTAGATCCT TTTAAATTAA AAATGAAGTT | 6780 | |
| | TTAAATCAAT CTAAAGTATA TATGAGTAAA CTTGGTCTGA CAGTTACCAA TGCTTAATCA | 6840 | |
| | GTGAGGCACC TATCTCAGCG ATCTGTCTAT TTCGTTCATC CATAGTTGCC TGACTCCCCG | 6900 | |
| | TCGTGTAGAT AACTACGATA CGGGAGGGCT TACCATCTGG CCCCAGTGCT GCAATGATAC | 6960 | |
| | CGCGAGAGCC ACGCTCACCG GCTCCAGATT TATCAGCAAT AAACCAGCCA GCCGGAAGGG | 7020 | |
| | CCGAGCGCAG AAGTGGTCCT GCAACTTTAT CCGCCTCCAT CCAGTCTATT AATTGTTGCC | 7080 | |
| | GGGAAGCTAG AGTAAGTAGT TCGCCAGTTA ATAGTTTGCG CAACGTTGTT GCCATTGCTA | 7140 | |
| | CAGGCATCGT GGTGTCACGC TCGTCGTTTG GTATGGCTTC ATTCAGCTCC GGTTCCCAAC | 7200 | |
| | GATCAAGGCG AGTTACATGA TCCCCCATGT TGTGCAAAAA AGCGGTTAGC TCCTTCGGTC | 7260 | |
| | CTCCGATCGT TGTCAGAAGT AAGTTGGCCG CAGTGTTATC ACTCATGGTT ATGGCAGCAC | 7320 | |
| | TGCATAATTC TCTTACTGTC ATGCCATCCG TAAGATGCTT TTCTGTGACT GGTGAGTACT | 7380 | |
| | CAACCAAGTC ATTCTGAGAA TAGTGTATGC GGCGACCGAG TTGCTCTTGC CCGGCGTCAA | 7440 | |
| | TACGGGATAA TACCGCGCCA CATAGCAGAA CTTTAAAAGT GCTCATCATT GGAAAACGTT | 7500 | |
| | CTTCGGGGCG AAAACTCTCA AGGATCTTAC CGCTGTTGAG ATCCAGTTCG ATGTAACCCA | 7560 | |
| | CTCGTGCACC CAACTGATCT TCAGCATCTT TTACTTTCAC CAGCGTTTCT GGGTGAGCAA | 7620 | |
| | AAACAGGAAG GCAAAATGCC GCAAAAAAGG GAATAAGGGC GACACGGAAA TGTTGAATAC | 7680 | |
| | TCATACTCTT CCTTTTTCAA TATTATTGAA GCATTTATCA GGGTTATTGT CTCATGAGCG | 7740 | |
| | GATACATATT TGAATGTATT TAGAAAAATA AACAAATAGG GGTTCCGCGC ACATTTCCCC | 7800 | |
| | GAAAAGTGCC AGATACCTGA AACAAAACCC ATCGTACGGC CAAGGAAGTC TCCAATAACT | 7860 | |
| | GTGATCCACC ACAAGCGCCA GGGTTTTCCC AGTCACGACG TTGTAAAACG ACGGCCAGTC | 7920 | |
| | ATGCATAATC CGCACGCATC TGGAATAAGG AAGTGCCATT CCGCCTGACC T | 7971 | |
| T2A Variant | GSGEGRGSLL TCGDVEENPG P | 21 | 87 |
| (amino acid) | | | |
| T2A Variant | GGCAGCGGCG AAGGCAGAGG ATCTCTGCTG ACATGTGGCG ACGTGGAAGA GAACCCCGGA | 60 | 88 |
| (nucleic acid) | CCT | 63 | |
| HSV | MASYPCHQHA SAFDQAARSR GHSNRRTALR PRRQQEATEV RLEQKMPTLL RVYIDGPHGM | 60 | 89 |
| Thymidine | GKTTTTQLLV ALGSRDDIVY VPEPMTYWQV LGASETIANI YTTQHRLDQG EISAGDAAVV | 120 | |
| Kinase | MTSAQITMGM PYAVTDAVLA PHIGGEAGSS HAPPPALTLI FDRHPIAHLL CYPAARYLMG | 180 | |
| A168H | SMTPQAVLAF VALIPPTLPG TNIVLGALPE DRHIDRLAKR QRPGERLDLA MLAAIRRVYG | 240 | |
| (amino acid) | LLANTVRYLQ GGGSWREDWG QLSGTAVPPQ GAEPQSNAGP RPHIGDTLFT LFRAPELLAP | 300 | |
| | NGDLYNVFAW ALDVLAKRLR PMHVFILDYD QSPAGCRDAL LQLTSGMVQT HVTTPGSIPT | 360 | |
| | ICDLARTFAR EMGEAN | 376 | |
| HSV | ATGGCCAGCT ATCCTTGTCA CCAGCACGCC AGCGCCTTTG ATCAGGCCGC AAGATCTAGA | 60 | 90 |
| Thymidine | GGCCACAGCA ACAGAAGAAC AGCCCTGCGG CCTCGGAGAC AGCAAGAGGC TACAGAAGTT | 120 | |
| Kinase | CGGCTGGAAC AGAAGATGCC CACACTGCTG CGGGTGTACA TCGATGGCCC TCACGGCATG | 180 | |
| A168H | GGCAAGACCA CCACAACACA GCTGCTGGTG GCCCTGGGCA GCAGAGATGA TATCGTGTAC | 240 | |
| (nucleic acid) | GTGCCCGAGC CTATGACCTA CTGGCAGGTT CTGGGAGCCA GCGAGACAAT CGCCAACATC | 300 | |
| | TACACCACAC AGCACCGGCT GGATCAGGGC GAAATTTCTG CTGGCGACGC CGCCGTGGTT | 360 | |
| | ATGACATCTG CCCAGATCAC CATGGGCATG CCTTACGCCG TGACAGATGC TGTGCTGGCC | 420 | |
| | CCTCACATTG GCGGAGAAGC CGGATCTTCT CATGCCCCTC CACCAGCTCT GACCCTGATC | 480 | |
| | TTCGACAGAC ACCCTATCGC CCATCTGCTG TGTTATCCTG CCGCCAGATA CCTGATGGGC | 540 | |
| | AGCATGACAC CTCAGGCCGT GCTGGCTTTC GTGGCCCTGA TTCCTCCTAC ACTGCCCGGC | 600 | |
| | ACCAATATCG TGCTGGGAGC CCTGCCTGAG GACCGGCACA TTGATAGACT GGCCAAGAGA | 660 | |
| | CAGCGGCCTG GCGAGAGACT GGATCTGGCT ATGCTGGCCG CCATCAGAAG AGTGTACGGC | 720 | |
| | CTGCTGGCCA ACACCGTGCG GTATCTTCAA GGCGGCGGAT CTTGGAGAGA GGACTGGGGA | 780 | |
| | CAACTGAGCG GCACAGCAGT TCCTCCACAA GGCGCTGAGC CTCAGTCTAA CGCTGGACCC | 840 | |
| | AGACCTCACA TCGGCGACAC CCTGTTTACC CTGTTCAGAG CCCCTGAGCT GCTGGCTCCT | 900 | |
| | AACGGCGACC TGTACAACGT GTTCGCCTGG GCTCTTGACG TGCTGGCAAA AAGACTGCGG | 960 | |
| | CCCATGCACG TGTTCATCCT GGACTACGAT CAGTCCCCTG CCGGCTGTAG AGATGCTCTG | 1020 | |
| | CTGCAGCTGA CAAGCGGCAT GGTGCAGACC CACGTTACAA CCCCTGGCAG CATCCCCACC | 1080 | |
| | ATCTGTGACC TGGCCAGAAC CTTCGCCAGA GAGATGGGAG AAGCCAAC | 1128 | |
| CD52 (amino | MKRFLFLLLT ISLLVMVQIQ TGLSGQNDTS QTSSPSASSN ISGGIFLFFV ANAIIHLFCF | 60 | 91 |
| acid) | S | 61 | |
| CD52 | ATGAAGAGGT TCCTGTTCCT GCTGCTGACC ATCAGCCTGC TGGTGATGGT GCAGATCCAG | 60 | 92 |
| (nucleic acid) | ACCGGCCTGA GCGGCCAGAA CGACACCAGC CAGACCAGCA GCCCCAGCGC CAGCAGCAAC | 120 | |
| | ATCAGCGGCG GCATCTTCCT GTTCTTCGTG GCCAACGCCA TCATCCACCT GTTCTGCTTC | 180 | |
| | AGGTGA | 186 | |
| WT HSV- | MASYPCHQHA SAFDQAARSR GHSNRRTALR PRRQQEATEV RLEQKMPTLL RVYIDGPHGM | 60 | 93 |
| TK-T2A- | GKTTTTQLLV ALGSRDDIVY VPEPMTYWQV LGASETIANI YTTQHRLDQG EISAGDAAVV | 120 | |
| PSMA | MTSAQITMGM PYAVTDAVLA PHIGGEAGSS HAPPPALTLI FDRHPIAALL CYPAARYLMG | 180 | |
| (N9del) | SMTPQAVLAF VALIPPTLPG TNIVLGALPE DRHIDRLAKR QRPGERLDLA MLAAIRRVYG | 240 | |
| (amino acid) | LLANTVRYLQ GGGSWREDWG QLSGTAVPPQ GAEPQSNAGP RPHIGDTLFT LFRAPELLAP | 300 | |
| | NGDLYNVFAW ALDVLAKRLR PMHVFILDYD QSPAGCRDAL LQLTSGMVQT HVTTPGSIPT | 360 | |
| | ICDLARTFAR EMGEANGSGE GRGSLLTCGD VEENPGPMWN LLARRPRWLC AGALVLAGGF | 420 | |
| | FLLGFLFGWF IKSSNEATNI TPKHNMKAFL DELKAENIKK FLYNFTQIPH LAGTEQNFQL | 480 | |
| | AKQIQSQWKE FGLDSVELAH YDVLLSYPNK THPNYISIIN EDGNEIFNTS LFEPPPPGYE | 540 | |
| | NVSDIVPPFS AFSPQGMPEG DLVYVNYART EDFFKLERDM KINCSGKIVI ARYGKVFRGN | 600 | |
| | KVKNAQLAGA KGVILYSDPA DYFAPGVKSY PDGWNLPGGG VQRGNILNLN GAGDPLTPGY | 660 | |
| | PANEYAYRRG IAEAVGLPSI PVHPIGYYDA QKLLEKMGGS APPDSSWRGS LKVPYNVGPG | 720 | |
| | FTGNFSTQKV KMHIHSTNEV TRIYNVIGTL RGAVEPDRYV ILGGHRDSWV FGGIDPQSGA | 780 | |
| | AVVHEIVRSF GTLKKEGWRP RRTILFASWD AEEFGLLGST EWAEENSRLL QERGVAYINA | 840 | |
| | DSSIEGNYTL RVDCTPLMYS LVHNLTKELK SPDEGFEGKS LYESWTKKSP SPEFSGMPRI | 900 | |
| | SKLGSGNDFE VFFQRLGIAS GRARYTKNWE TNKFSGYPLY HSVYETYELV EKFYDPMFKY | 960 | |
| | HLTVAQVRGG MVFELANSIV LPFDCRDYAV VLRKYADKIY SISMKHPQEM KTYSVSFDSL | 1020 | |
| | FSAVKNFTEI ASKFSERLQD FDKSNPIVLR MMNDQLMFLE RAFIDPLGLP DRPFYRHVIY | 1080 | |
| | APSSHNKYAG ESFPGIYDAL FDIESKVDPS KAWGEVKRQI YVAAFTVQAA AETLSEVA | 1138 | |
| (A168H) | MASYPCHQHA SAFDQAARSR GHSNRRTALR PRRQQEATEV RLEQKMPTLL RVYIDGPHGM | 60 | 94 |
| HSV-TK- | GKTTTTQLLV ALGSRDDIVY VPEPMTYWQV LGASETIANI YTTQHRLDQG EISAGDAAVV | 120 | |
| T2A-PSMA | MTSAQITMGM PYAVTDAVLA PHIGGEAGSS HAPPPALTLI FDRHPIAHLL CYPAARYLMG | 180 | |
| (N9del) | SMTPQAVLAF VALIPPTLPG TNIVLGALPE DRHIDRLAKR QRPGERLDLA MLAAIRRVYG | 240 | |
| (amino acid) | LLANTVRYLQ GGGSWREDWG QLSGTAVPPQ GAEPQSNAGP RPHIGDTLFT LFRAPELLAP | 300 | |
| | NGDLYNVFAW ALDVLAKRLR PMHVFILDYD QSPAGCRDAL LQLTSGMVQT HVTTPGSIPT | 360 | |
| | ICDLARTFAR EMGEANGSGE GRGSLLTCGD VEENPGPMWN LLARRPRWLC AGALVLAGGF | 420 | |
| | FLLGFLFGWF IKSSNEATNI TPKHNMKAFL DELKAENIKK FLYNFTQIPH LAGTEQNFQL | 480 | |
| | AKQIQSQWKE FGLDSVELAH YDVLLSYPNK THPNYISIIN EDGNEIFNTS LFEPPPPGYE | 540 | |
| | NVSDIVPPFS AFSPQGMPEG DLVYVNYART EDFFKLERDM KINCSGKIVI ARYGKVFRGN | 600 | |
| | KVKNAQLAGA KGVILYSDPA DYFAPGVKSY PDGWNLPGGG VQRGNILNLN GAGDPLTPGY | 660 | |
| | PANEYAYRRG IAEAVGLPSI PVHPIGYYDA QKLLEKMGGS APPDSSWRGS LKVPYNVGPG | 720 | |
| | FTGNFSTQKV KMHIHSTNEV TRIYNVIGTL RGAVEPDRYV ILGGHRDSWV FGGIDPQSGA | 780 | |
| | AVVHEIVRSF GTLKKEGWRP RRTILFASWD AEEFGLLGST EWAEENSRLL QERGVAYINA | 840 | |
| | DSSIEGNYTL RVDCTPLMYS LVHNLTKELK SPDEGFEGKS LYESWTKKSP SPEFSGMPRI | 900 | |
| | SKLGSGNDFE VFFQRLGIAS GRARYTKNWE TNKFSGYPLY HSVYETYELV EKFYDPMFKY | 960 | |
| | HLTVAQVRGG MVFELANSIV LPFDCRDYAV VLRKYADKIY SISMKHPQEM KTYSVSFDSL | 1020 | |
| | FSAVKNFTEI ASKFSERLQD FDKSNPIVLR MMNDQLMFLE RAFIDPLGLP DRPFYRHVIY | 1080 | |
| | APSSHNKYAG ESFPGIYDAL FDIESKVDPS KAWGEVKRQI YVAAFTVQAA AETLSEVA | 1138 | |
| (A168H) | ATGGCCAGCT ATCCTTGTCA CCAGCACGCC AGCGCCTTTG ATCAGGCCGC AAGATCTAGA | 60 | 95 |
| HSV-TK- | GGCCACAGCA ACAGAAGAAC AGCCCTGCGG CCTCGGAGAC AGCAAGAGGC TACAGAAGTT | 120 | |
| T2A-PSMA | CGGCTGGAAC AGAAGATGCC CACACTGCTG CGGGTGTACA TCGATGGCCC TCACGGCATG | 180 | |
| (N9del) | GGCAAGACCA CCACAACACA GCTGCTGGTG GCCCTGGGCA GCAGAGATGA TATCGTGTAC | 240 | |
| (nucleic acid) | GTGCCCGAGC CTATGACCTA CTGGCAGGTT CTGGGAGCCA GCGAGACAAT CGCCAACATC | 300 | |
| | TACACCACAC AGCACCGGCT GGATCAGGGC GAAATTTCTG CTGGCGACGC CGCCGTGGTT | 360 | |
| | ATGACATCTG CCCAGATCAC CATGGGCATG CCTTACGCCG TGACAGATGC TGTGCTGGCC | 420 | |
| | CCTCACATTG GCGGAGAAGC CGGATCTTCT CATGCCCCTC CACCAGCTCT GACCCTGATC | 480 | |
| | TTCGACAGAC ACCCTATCGC CCATCTGCTG TGTTATCCTG CCGCCAGATA CCTGATGGGC | 540 | |
| | AGCATGACAC CTCAGGCCGT GCTGGCTTTC GTGGCCCTGA TTCCTCCTAC ACTGCCCGGC | 600 | |
| | ACCAATATCG TGCTGGGAGC CCTGCCTGAG GACCGGCACA TTGATAGACT GGCCAAGAGA | 660 | |
| | CAGCGGCCTG GCGAGAGACT GGATCTGGCT ATGCTGGCCG CCATCAGAAG AGTGTACGGC | 720 | |
| | CTGCTGGCCA ACACCGTGCG GTATCTTCAA GGCGGCGGAT CTTGGAGAGA GGACTGGGGA | 780 | |
| | CAACTGAGCG GCACAGCAGT TCCTCCACAA GGCGCTGAGC CTCAGTCTAA CGCTGGACCC | 840 | |
| | AGACCTCACA TCGGCGACAC CCTGTTTACC CTGTTCAGAG CCCCTGAGCT GCTGGCTCCT | 900 | |
| | AACGGCGACC TGTACAACGT GTTCGCCTGG GCTCTTGACG TGCTGGCAAA AAGACTGCGG | 960 | |
| | CCCATGCACG TGTTCATCCT GGACTACGAT CAGTCCCCTG CCGGCTGTAG AGATGCTCTG | 1020 | |
| | CTGCAGCTGA CAAGCGGCAT GGTGCAGACC CACGTTACAA CCCCTGGCAG CATCCCCACC | 1080 | |
| | ATCTGTGACC TGGCCAGAAC CTTCGCCAGA GAGATGGGAG AAGCCAACGG CAGCGGCGAA | 1140 | |
| | GGCAGAGGAT CTCTGCTGAC ATGTGGCGAC GTGGAAGAGA ACCCCGGACC TATGTGGAAC | 1200 | |
| | CTGCTGGCTA GACGGCCCAG ATGGCTTTGT GCTGGTGCTC TGGTTCTGGC TGGCGGCTTT | 1260 | |
| | TTCCTGCTGG GCTTCCTGTT TGGCTGGTTC ATCAAGAGCA GCAAGGAGGC CACCAACATC | 1320 | |
| | ACCCCTAAGC ACAACATGAA GGCCTTTCTG GACGAGCTGA AGGCCGAGAA TATCAAGAAG | 1380 | |
| | TTCCTCTACA ACTTCACGCA GATCCCTCAC CTGGCCGGCA CCGAGCAGAA TTTTCAGCTG | 1440 | |
| | GCCAAGCAGA TCCAGAGCCA GTGGAAAGAG TTCGGCCTGG ACTCTGTGGA ACTGGCCCAC | 1500 | |
| | TACGATGTGC TGCTGAGCTA CCCCAACAAG ACACACCCCA ACTACATCAG CATCATCAAC | 1560 | |
| | GAGGACGGCA ACGAGATCTT CAACACCAGC CTGTTCGAGC CTCCACCTCC TGGCTACGAG | 1620 | |
| | AACGTGTCCG ATATCGTGCC TCCATTCAGC GCTTTCAGCC CTCAAGGGAT GCCTGAGGGC | 1680 | |
| | GATCTGGTGT ACGTGAACTA CGCCAGAACC GAGGACTTCT TCAAGCTGGA ACGGGACATG | 1740 | |
| | AAGATCAACT GCTCCGGCAA GATCGTGATC GCCCGCTACG GCAAAGTGTT CCGGGGCAAC | 1800 | |
| | AAAGTGAAGA ACGCCCAGCT GGCAGGCGCC AAAGGCGTGA TCCTGTATAG CGACCCCGCC | 1860 | |
| | GACTATTTTG CCCCTGGCGT GAAGTCTTAC CCCGACGGCT GGAATCTTCC TGGTGGCGGA | 1920 | |
| | GTGCAGAGAG GCAACATCCT GAACCTTAAC GGCGCAGGCG ACCCTCTGAC ACCTGGCTAT | 1980 | |
| | CCTGCCAATG AGTACGCCTA CAGACGGGGA ATTGCCGAGG CTGTGGGACT GCCTTCTATC | 2040 | |
| | CCTGTGCACC CCATCGGCTA CTACGACGCC CAGAAACTGC TGGAAAAGAT GGGCGGAAGC | 2100 | |
| | GCCCCTCCTG ACTCTAGTTG GAGAGGCTCT CTGAAGGTGC CCTACAATGT CGGCCCAGGC | 2160 | |
| | TTCACCGGCA ACTTCAGGAC CCAGAAAGTG AAAATGCACA TCCACAGCAC CAACGAAGTG | 2220 | |
| | ACCCGGATCT ACAACGTGAT CGGCACACTG AGAGGCGCCG TGGAACCCGA CAGATATGTG | 2280 | |
| | ATCCTCGGCG GCCACAGAGA TAGCTGGGTG TTCGGAGGAA TCGACCCTCA GTCTGGTGCC | 2340 | |
| | GCTGTGGTGC ACGAAATCGT GCGGTCTTTT GGCACCCTGA AGAAAGAAGG CTGGCGCCCC | 2400 | |
| | AGACGGACCA TCCTGTTTGC CTCTTGGGAC GCCGAGGAAT TCGGACTGCT GGGATCTACA | 2460 | |
| | GAGTGGGCCG AAGAGAACAG CAGACTGCTG CAAGAAAGAG GCGTGGCCTA CATCAACGCC | 2520 | |
| | GACAGCAGCA TCGAGGGCAA CTACACCCTG AGAGTGGACT GCACCCCTCT GATGTACAGC | 2580 | |
| | CTGGTGCACA ACCTGACCAA AGAGCTGAAG TCCCCTGACG AGGGCTTTGA GGGCAAGAGC | 2640 | |
| | CTGTACGAGA GCTGGACCAA GAAGTCCCCA TCTCCTGAGT TCAGCGGCAT GCCCCGGATC | 2700 | |
| | TCTAAGCTCG GCTCTGGCAA CGACTTCGAG GTGTTCTTCC AGCGGCTGGG AATCGCTTCT | 2760 | |
| | GGCAGAGCCA GATACACCAA GAACTGGGAG ACAAACAAGT TCTCCGGCTA TCCCCTGTAC | 2820 | |
| | CACAGCGTGT ACGAGACATA CGAGCTGGTC GAGAAGTTCT ACGACCCCAT GTTCAAGTAC | 2880 | |
| | CACCTGACAG TGGCCCAAGT GCGCGGAGGC ATGGTGTTCG AACTGGCCAA TAGCATCGTG | 2940 | |
| | CTGCCCTTCG ACTGCAGAGA CTATGCCGTG GTGCTGCGGA AGTACGCCGA TAAGATCTAC | 3000 | |
| | AGCATCAGCA TGAAGCACCC GCAAGAGATG AAGACCTACA GCGTGTCCTT CGATAGCCTG | 3060 | |
| | TTCAGCGCCG TGAAGAACTT CACCGAGATC GCCAGCAAGT TCAGCGAGCG GCTGCAGGAC | 3120 | |
| | TTCGACAAGA GCAACCCAAT CGTGCTGAGG ATGATGAACG ACCAGCTGAT GTTCCTGGAA | 3180 | |
| | CGCGCCTTCA TCGACCCACT GGGCTTGCCC GATAGACCCT TCTACCGGCA CGTGATCTAT | 3240 | |
| | GCCCCTAGCA GCCACAACAA ATACGCCGGC GAGAGCTTCC CCGGCATCTA CGATGCCCTG | 3300 | |
| | TTCGACATCG AGAGCAAGGT GGACCCATCT AAGGCCTGGG GCGAAGTGAA GCGGCAGATC | 3360 | |
| | TACGTGGCCG CATTCACAGT GCAGGCTGCC GCCGAAACAC TGTCTGAAGT GGCTTGA | 3417 | |
| WT HSV- | MASYPCHQHA SAFDQAARSR GHSNRRTALR PRRQQEATEV RLEQKMPTLL RVYIDGPHGM | 60 | 96 |
| TK-T2A- | GKTTTTQLLV ALGSRDDIVY VPEPMTYWQV LGASETIANI YTTQHRLDQG EISAGDAAVV | 120 | |
| CD52 (amino | MTSAQITMGM PYAVTDAVLA PHIGGEAGSS HAPPPALTLI FDRHPIAALL CYPAARYLMG | 180 | |
| acid) | SMTPQAVLAF VALIPPTLPG TNIVLGALPE DRHIDRLAKR QRPGERLDLA MLAAIRRVYG | 240 | |
| | LLANTVRYLQ GGGSWREDWG QLSGTAVPPQ GAEPQSNAGP RPHIGDTLFT LFRAPELLAP | 300 | |
| | NGDLYNVFAW ALDVLAKRLR PMHVFILDYD QSPAGCRDAL LQLTSGMVQT HVTTPGSIPT | 360 | |
| | ICDLARTFAR EMGEANGSGE GRGSLLTCGD VEENPGPMKR FLFLLLTISL LVMVQIQTGL | 420 | |
| | SGQNDTSQTS SPSASSNISG GIFLFFVANA IIHLFCFS | 458 | |
| (A168H) | MASYPCHQHA SAFDQAARSR GHSNRRTALR PRRQQEATEV RLEQKMPTLL RVYIDGPHGM | 60 | 97 |
| HSV-TK- | GKTTTTQLLV ALGSRDDIVY VPEPMTYWQV LGASETIANI YTTQHRLDQG EISAGDAAVV | 120 | |
| T2A-CD52 | MTSAQITMGM PYAVTDAVLA PHIGGEAGSS HAPPPALTLI FDRHPIAHLL CYPAARYLMG | 180 | |
| (amino acid) | SMTPQAVLAF VALIPPTLPG TNIVLGALPE DRHIDRLAKR QRPGERLDLA MLAAIRRVYG | 240 | |
| | LLANTVRYLQ GGGSWREDWG QLSGTAVPPQ GAEPQSNAGP RPHIGDTLFT LFRAPELLAP | 300 | |
| | NGDLYNVFAW ALDVLAKRLR PMHVFILDYD QSPAGCRDAL LQLTSGMVQT HVTTPGSIPT | 360 | |
| | ICDLARTFAR EMGEANGSGE GRGSLLTCGD VEENPGPMKR FLFLLLTISL LVMVQIQTGL | 420 | |
| | SGQNDTSQTS SPSASSNISG GIFLFFVANA IIHLFCFS | 458 | |
| (A168H) | ATGGCCAGCT ATCCTTGTCA CCAGCACGCC AGCGCCTTTG ATCAGGCCGC AAGATCTAGA | 60 | 98 |
| HSV-TK- | GGCCACAGCA ACAGAAGAAC AGCCCTGCGG CCTCGGAGAC AGCAAGAGGC TACAGAAGTT | 120 | |
| T2A-CD52 | CGGCTGGAAC AGAAGATGCC CACACTGCTG CGGGTGTACA TCGATGGCCC TCACGGCATG | 180 | |
| (nucleic acid) | GGCAAGACCA CCACAACACA GCTGCTGGTG GCCCTGGGCA GCAGAGATGA TATCGTGTAC | 240 | |
| | GTGCCCGAGC CTATGACCTA CTGGCAGGTT CTGGGAGCCA GCGAGACAAT CGCCAACATC | 300 | |
| | TACACCACAC AGCACCGGCT GGATCAGGGC GAAATTTCTG CTGGCGACGC CGCCGTGGTT | 360 | |
| | ATGACATCTG CCCAGATCAC CATGGGCATG CCTTACGCCG TGACAGATGC TGTGCTGGCC | 420 | |
| | CCTCACATTG GCGGAGAAGC CGGATCTTCT CATGCCCCTC CACCAGCTCT GACCCTGATC | 480 | |
| | TTCGACAGAC ACCCTATCGC CCATCTGCTG TGTTATCCTG CCGCCAGATA CCTGATGGGC | 540 | |
| | AGCATGACAC CTCAGGCCGT GCTGGCTTTC GTGGCCCTGA TTCCTCCTAC ACTGCCCGGC | 600 | |
| | ACCAATATCG TGCTGGGAGC CCTGCCTGAG GACCGGCACA TTGATAGACT GGCCAAGAGA | 660 | |
| | CAGCGGCCTG GCGAGAGACT GGATCTGGCT ATGCTGGCCG CCATCAGAAG AGTGTACGGC | 720 | |
| | CTGCTGGCCA ACACCGTGCG GTATCTTCAA GGCGGCGGAT CTTGGAGAGA GGACTGGGGA | 780 | |
| | CAACTGAGCG GCACAGCAGT TCCTCCACAA GGCGCTGAGC CTCAGTCTAA CGCTGGACCC | 840 | |
| | AGACCTCACA TCGGCGACAC CCTGTTTACC CTGTTCAGAG CCCCTGAGCT GCTGGCTCCT | 900 | |
| | AACGGCGACC TGTACAACGT GTTCGCCTGG GCTCTTGACG TGCTGGCAAA AAGACTGCGG | 960 | |
| | CCCATGCACG TGTTCATCCT GGACTACGAT CAGTCCCCTG CCGGCTGTAG AGATGCTCTG | 1020 | |
| | CTGCAGCTGA CAAGCGGCAT GGTGCAGACC CACGTTACAA CCCCTGGCAG CATCCCCACC | 1080 | |
| | ATCTGTGACC TGGCCAGAAC CTTCGCCAGA GAGATGGGAG AAGCCAACGG CAGCGGCGAA | 1140 | |
| | GGCAGAGGAT CTCTGCTGAC ATGTGGCGAC GTGGAAGAGA ACCCCGGACC TATGAAGAGG | 1200 | |
| | TTCCTGTTCC TGCTGCTGAC CATCAGCCTG CTGGTGATGG TGCAGATCCA GACCGGCCTG | 1260 | |
| | AGCGGCCAGA ACGACACCAG CCAGACCAGC AGCCCCAGCG CCAGCAGCAA CATCAGCGGC | 1320 | |
| | GGCATCTTCC TGTTCTTCGT GGCCAACGCC ATCATCCACC TGTTCTGCTT CAGCTGA | 1377 | |

### (3) Nucleic Acids Encoding an HLA Construct

In another general aspect, the invention relates to an isolated nucleic acid encoding an HLA construct useful for an invention according to embodiments of the application. It will be appreciated by those skilled in the art that the coding sequence of an HLA construct can be changed (e.g., replaced, deleted, inserted, etc.) without changing the amino acid sequence of the protein. Accordingly, it will be understood by those skilled in the art that nucleic acid sequences encoding an HLA construct of the application can be altered without changing the amino acid sequences of the proteins.

In certain embodiments, the isolated nucleic acid encodes an HLA construct comprising a signal peptide, such as an HLA-G signal peptide, operably linked to an HLA coding sequence, such as a coding sequence of a mature B2M, and/or a mature HLA-E. In some embodiments, the HLA coding sequence encodes the HLA-G and B2M, which are operably linked by a 4X GGGGS linker, and/or the B2M and HLA-E, which are operably linked by a 3X GGGGS linker. In a particular embodiment, the isolated nucleic acid encoding the HLA construct comprises a polynucleotide sequence at least 90%, such as at least 90%, 91%, 92%, 93%, 94%, 95%, 96%, 97%, 98% or 100%, identical to SEQ ID NO: 67, preferably the polynucleotide sequence of SEQ ID NO: 67. In another embodiment, the isolated nucleic acid encoding the HLA construct comprises a polynucleotide sequence at least 90%, such as at least 90%, 91%, 92%, 93%, 94%, 95%, 96%, 97%, 98% or 100%, identical to SEQ ID NO: 70, preferably the polynucleotide sequence of SEQ ID NO: 70.

In another general aspect, the application provides a vector comprising a polynucleotide sequence encoding a HLA construct useful for an invention according to embodiments of the application. Any vector known to those skilled in the art in view of the present disclosure can be used, such as a plasmid, a cosmid, a phage vector or a viral vector. In some embodiments, the vector is a recombinant expression vector such as a plasmid. The vector can include any element to establish a conventional function of an expression vector, for example, a promoter, ribosome binding element, terminator, enhancer, selection marker, and origin of replication. The promoter can be a constitutive, inducible, or repressible promoter. A number of expression vectors capable of delivering nucleic acids to a cell are known in the art and can be used herein for production of a HLA construct in the cell. Conventional cloning techniques or artificial gene synthesis can be used to generate a recombinant expression vector according to embodiments of the application.

In a particular aspect, the application provides vectors for targeted integration of a HLA construct useful for an invention according to embodiments of the application. In certain embodiments, the vector comprises an exogenous polynucleotide having, in the 5′ to 3′ order, (a) a promoter; (b) a polynucleotide sequence encoding an HLA construct; and (c) a terminator/polyadenylation signal.

In certain embodiments, the promoter is a CAG promoter. In certain embodiments, the CAG promoter comprises the polynucleotide sequence at least 90%, such as at least 90%, 91%, 92%, 93%, 94%, 95%, 96%, 97%, 98% or 100%, identical to SEQ ID NO: 63. Other promoters can also be used, examples of which include, but are not limited to, EFla, UBC, CMV, SV40, PGK1, and human beta actin.

In certain embodiments, the terminator/polyadenylation signal is a SV40 signal. In certain embodiments, the SV40 signal comprises the polynucleotide sequence at least 90%, such as at least 90%, 91%, 92%, 93%, 94%, 95%, 96%, 97%, 98% or 100%, identical to SEQ ID NO: 64. Other terminator sequences can also be used, examples of which include, but are not limited to BGH, hGH, and PGK.

In certain embodiments, a polynucleotide sequence encoding a HLA construct comprises a signal peptide, such as a HLA-G signal peptide, a mature B2M, and a mature HLA-E, wherein the HLA-G and B2M are operably linked by a 4X GGGGS linker (SEQ ID NO: 31) and the B2M transgene and HLA-E are operably linked by a 3X GGGGS linker (SEQ ID NO: 25). In particular embodiments, the HLA construct comprises the polynucleotide sequence at least 90%, such as at least 90%, 91%, 92%, 93%, 94%, 95%, 96%, 97%, 98% or 100%, identical to SEQ ID NO: 67, preferably the polynucleotide sequence of SEQ ID NO: 67. In another embodiment, the HLA construct comprises the polynucleotide sequence at least 90%, such as at least 90%, 91%, 92%, 93%, 94%, 95%, 96%, 97%, 98% or 100%, identical to SEQ ID NO: 70, preferably the polynucleotide sequence of SEQ ID NO: 70.

In some embodiment, the vector further comprises a left homology arm and a right homology arm flanking the exogenous polynucleotide.

In certain embodiments, the left homology arm comprises the polynucleotide sequence at least 90%, such as at least 90%, 91%, 92%, 93%, 94%, 95%, 96%, 97%, 98% or 100%, identical to SEQ ID NO: 81. In certain embodiments, the right homology arm comprises the polynucleotide sequence at least 90%, such as at least 90%, 91%, 92%, 93%, 94%, 95%, 96%, 97%, 98% or 100%, identical to SEQ ID NO: 82.

In a particular embodiment, the vector comprises a polynucleotide sequence at least 85%, such as at least 85%, 86%, 87%, 88%, 89%, 90%, 91%, 92%, 93%, 94%, 95%, 96%, 97%, 98% or 100%, identical to SEQ ID NO: 85, preferably the polynucleotide sequence of SEQ ID NO: 83.

### (4) Host Cells

In another general aspect, the application provides a host cell comprising a vector of the application and/or an isolated nucleic acid encoding a construct of the application. Any host cell known to those skilled in the art in view of the present disclosure can be used for recombinant expression of exogenous polynucleotides of the application. According to particular embodiments, the recombinant expression vector is transformed into host cells by conventional methods such as chemical transfection, heat shock, or electroporation, where it is stably integrated into the host cell genome such that the recombinant nucleic acid is effectively expressed.

Examples of host cells include, for example, recombinant cells containing a vector or isolated nucleic acid of the application useful for the production of a vector or construct of interest; or an engineered iPSC or derivative cell thereof containing one or more isolated nucleic acids of the application, preferably integrated at one or more chromosomal loci. A host cell of an isolated nucleic acid of the application can also be an immune effector cell, such as a T cell, comprising the one or more isolated nucleic acids of the application. The immune effector cell can be obtained by differentiation of an engineered iPSC of the application. Any suitable method in the art can be used for the differentiation in view of the present disclosure. The immune effector cell can also be obtained transfecting an immune effector cell with one or more isolated nucleic acids of the application.

### IX. Compositions

In another general aspect, the application provides a composition comprising an isolated polynucleotide of the application, a host cell and/or an iPSC or derivative cell thereof of the application.

In certain embodiments, the composition further comprises one or more therapeutic agents selected from the group consisting of a peptide, a cytokine, a checkpoint inhibitor, a mitogen, a growth factor, a small RNA, a dsRNA (double stranded RNA), siRNA, oligonucleotide, mononuclear blood cells, a vector comprising one or more polynucleic acids of interest, an antibody, a chemotherapeutic agent or a radioactive moiety, or an immunomodulatory drug (IMiD).

In certain embodiments, the composition is a pharmaceutical composition comprising an isolated polynucleotide of the application, a host cell and/or an iPSC or derivative cell thereof of the application and a pharmaceutically acceptable carrier. The term “pharmaceutical composition” as used herein means a product comprising an isolated polynucleotide of the application, an isolated polypeptide of the application, a host cell of the application, and/or an iPSC or derivative cell thereof of the application together with a pharmaceutically acceptable carrier. Polynucleotides, polypeptides, host cells, and/or iPSCs or derivative cells thereof of the application and compositions comprising them are also useful in the manufacture of a medicament for therapeutic applications mentioned herein.

As used herein, the term “carrier” refers to any excipient, diluent, filler, salt, buffer, stabilizer, solubilizer, oil, lipid, lipid containing vesicle, microsphere, liposomal encapsulation, or other material well known in the art for use in pharmaceutical formulations. It will be understood that the characteristics of the carrier, excipient or diluent will depend on the route of administration for a particular application. As used herein, the term “pharmaceutically acceptable carrier” refers to a non-toxic material that does not interfere with the effectiveness of a composition described herein or the biological activity of a composition described herein. According to particular embodiments, in view of the present disclosure, any pharmaceutically acceptable carrier suitable for use in a polynucleotide, polypeptide, host cell, and/or iPSC or derivative cell thereof can be used.

The formulation of pharmaceutically active ingredients with pharmaceutically acceptable carriers is known in the art, e.g., Remington: The Science and Practice of Pharmacy (e.g. 21st edition (2005), and any later editions). Non-limiting examples of additional ingredients include: buffers, diluents, solvents, tonicity regulating agents, preservatives, stabilizers, and chelating agents. One or more pharmaceutically acceptable carrier can be used in formulating the pharmaceutical compositions of the application.

### X. Methods of Use

In a general aspect, the application provides a method of eliminating a cell comprising a polynucleotide encoding a combined artificial cell death/reporter system polypeptide according embodiments of the application.

In certain embodiments, the method comprises contacting the cell with one or more agents that bind to the artificial cell death polypeptide to thereby induce death of the cell.

In certain embodiments, the one or more agents comprise acyclovir or a derivative thereof, or ganciclovir or a derivative thereof. In certain embodiments, the one or more agents further comprise an agent that binds to the PSMA extracellular domain or fragment thereof, such as a radioisotopic conjugate that binds to the PSMA polypeptide.

Also provided is a method of treating a disease or a condition in a subject in need thereof. The methods comprise administering to the subject in need thereof a therapeutically effective amount of cells of the application and/or a composition of the application. In certain embodiments, the disease or condition is cancer. The cancer can, for example, be a solid or a liquid cancer. The cancer, can, for example, be selected from the group consisting of a lung cancer, a gastric cancer, a colon cancer, a liver cancer, a renal cell carcinoma, a bladder urothelial carcinoma, a metastatic melanoma, a breast cancer, an ovarian cancer, a cervical cancer, a head and neck cancer, a pancreatic cancer, an endometrial cancer, a prostate cancer, a thyroid cancer, a glioma, a glioblastoma, and other solid tumors, and a non-Hodgkin's lymphoma (NHL), Hodgkin's lymphoma/disease (HD), an acute lymphocytic leukemia (ALL), a chronic lymphocytic leukemia (CLL), a chronic myelogenous leukemia (CML), a multiple myeloma (MM), an acute myeloid leukemia (AML), and other liquid tumors. In a preferred embodiment, the cancer is a non-Hodgkin's lymphoma (NHL).

According to embodiments of the application, the composition comprises a therapeutically effective amount of an isolated polynucleotide, an isolated polypeptide, a host cell, and/or an iPSC or derivative cell thereof. As used herein, the term “therapeutically effective amount” refers to an amount of an active ingredient or component that elicits the desired biological or medicinal response in a subject. A therapeutically effective amount can be determined empirically and in a routine manner, in relation to the stated purpose.

As used herein with reference to a cell of the application and/or a pharmaceutical composition of the application a therapeutically effective amount means an amount of the cells and/or the pharmaceutical composition that modulates an immune response in a subject in need thereof.

According to particular embodiments, a therapeutically effective amount refers to the amount of therapy which is sufficient to achieve one, two, three, four, or more of the following effects: (i) reduce or ameliorate the severity of the disease, disorder or condition to be treated or a symptom associated therewith; (ii) reduce the duration of the disease, disorder or condition to be treated, or a symptom associated therewith; (iii) prevent the progression of the disease, disorder or condition to be treated, or a symptom associated therewith; (iv) cause regression of the disease, disorder or condition to be treated, or a symptom associated therewith; (v) prevent the development or onset of the disease, disorder or condition to be treated, or a symptom associated therewith; (vi) prevent the recurrence of the disease, disorder or condition to be treated, or a symptom associated therewith; (vii) reduce hospitalization of a subject having the disease, disorder or condition to be treated, or a symptom associated therewith; (viii) reduce hospitalization length of a subject having the disease, disorder or condition to be treated, or a symptom associated therewith; (ix) increase the survival of a subject with the disease, disorder or condition to be treated, or a symptom associated therewith; (xi) inhibit or reduce the disease, disorder or condition to be treated, or a symptom associated therewith in a subject; and/or (xii) enhance or improve the prophylactic or therapeutic effect(s) of another therapy.

In particular embodiments, the cells of the invention are allogeneic to the patient being treated.

The therapeutically effective amount or dosage can vary according to various factors, such as the disease, disorder or condition to be treated, the means of administration, the target site, the physiological state of the subject (including, e.g., age, body weight, health), whether the subject is a human or an animal, other medications administered, and whether the treatment is prophylactic or therapeutic. Treatment dosages are optimally titrated to optimize safety and efficacy.

According to particular embodiments, the compositions described herein are formulated to be suitable for the intended route of administration to a subject. For example, the compositions described herein can be formulated to be suitable for intravenous, subcutaneous, or intramuscular administration.

The cells of the application and/or the pharmaceutical compositions of the application can be administered in any convenient manner known to those skilled in the art. For example, the cells of the application can be administered to the subject by aerosol inhalation, injection, ingestion, transfusion, implantation, and/or transplantation. The compositions comprising the cells of the application can be administered intrathecally, transarterially, subcutaneously, intradermaly, intratumorally, intranodally, intramedullary, intramuscularly, inrapleurally, by intravenous (i.v.) injection, or intraperitoneally. In certain embodiments, the cells of the application can be administered with or without lymphodepletion of the subject.

For use in glioblastoma, where the CAR cell is targeting one or more antigens expressed in glioblastoma cells, the immune effector cells expressing the combined artificial cell death/reporter system polypeptide may be administered intrathecally. Intrathecal administration is a route of administration for drugs via an injection into the spinal canal, or into the subarachnoid space so that it reaches the cerebrospinal fluid (CSF) and is useful in chemotherapy of glioblastoma patients. Administration of the drug in this manner avoids the drug being stopped by the blood brain barrier. When given by the intrathecal route the immune effector cells will be formulated in a manner such that they do not contain any preservative or other potentially harmful inactive ingredients that are sometimes found in standard injectable drug preparations. Use of the cells engineered with a reporter system as in the current invention has the advantage of potentially eliminating or reducing the need for biopsies since the physician will be able to image the cells to determine their biodistribution without having to biopsy.

The pharmaceutical compositions comprising cells of the application can be provided in sterile liquid preparations, typically isotonic aqueous solutions with cell suspensions, or optionally as emulsions, dispersions, or the like, which are typically buffered to a selected pH. The compositions can comprise carriers, for example, water, saline, phosphate buffered saline, and the like, suitable for the integrity and viability of the cells, and for administration of a cell composition.

Sterile injectable solutions can be prepared by incorporating cells of the application in a suitable amount of the appropriate solvent with various other ingredients, as desired. Such compositions can include a pharmaceutically acceptable carrier, diluent, or excipient such as sterile water, physiological saline, glucose, dextrose, or the like, that are suitable for use with a cell composition and for administration to a subject, such as a human. Suitable buffers for providing a cell composition are well known in the art. Any vehicle, diluent, or additive used is compatible with preserving the integrity and viability of the cells of the application.

The cells of the application and/or the pharmaceutical compositions of the application can be administered in any physiologically acceptable vehicle. A cell population comprising cells of the application can comprise a purified population of cells. Those skilled in the art can readily determine the cells in a cell population using various well-known methods. The ranges in purity in cell populations comprising genetically modified cells of the application can be from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. Dosages can be readily adjusted by those skilled in the art, for example, a decrease in purity could require an increase in dosage.

The cells of the application are generally administered as a dose based on cells per kilogram (cells/kg) of body weight of the subject to which the cells and/or pharmaceutical compositions comprising the cells are administered. Generally, the cell doses are in the range of about 10⁴ to about 10¹⁰ cells/kg of body weight, for example, about 10⁵ to about 10⁹, about 10⁵ to about 10⁸, about 10⁵ to about 10⁷, or about 10⁵ to about 10⁶, depending on the mode and location of administration. In general, in the case of systemic administration, a higher dose is used than in regional administration, where the immune cells of the application are administered in the region of a tumor and/or cancer. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 6×10⁸, 7×10⁴ to 1×10⁸, 8×10⁴ to 1×10⁸, 9×10⁴ to 1×10⁸, 1×10⁵ to 1×10⁸, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 2×10⁵ to 4×10⁶, 2×10⁵ to 3×10⁶, 2×10⁵ to 2×10⁶, 2×10⁵ to 1×10⁶, 3×10⁵ to 3×10⁶ cells/kg, and the like. Additionally, the dose can be adjusted to account for whether a single dose is being administered or whether multiple doses are being administered. The precise determination of what would be considered an effective dose can be based on factors individual to each subject.

As used herein, the terms “treat,” “treating,” and “treatment” are all intended to refer to an amelioration or reversal of at least one measurable physical parameter related to a cancer, which is not necessarily discernible in the subject, but can be discernible in the subject. The terms “treat,” “treating,” and “treatment,” can also refer to causing regression, preventing the progression, or at least slowing down the progression of the disease, disorder, or condition. In a particular embodiment, “treat,” “treating,” and “treatment” refer to an alleviation, prevention of the development or onset, or reduction in the duration of one or more symptoms associated with the disease, disorder, or condition, such as a tumor or more preferably a cancer. In a particular embodiment, “treat,” “treating,” and “treatment” refer to prevention of the recurrence of the disease, disorder, or condition. In a particular embodiment, “treat,” “treating,” and “treatment” refer to an increase in the survival of a subject having the disease, disorder, or condition. In a particular embodiment, “treat,” “treating,” and “treatment” refer to elimination of the disease, disorder, or condition in the subject.

The cells of the application and/or the pharmaceutical compositions of the application can be administered in combination with one or more additional therapeutic agents. In certain embodiments the one or more therapeutic agents are selected from the group consisting of a peptide, a cytokine, a checkpoint inhibitor, a mitogen, a growth factor, a small RNA, a dsRNA (double stranded RNA), siRNA, oligonucleotide, mononuclear blood cells, a vector comprising one or more polynucleic acids of interest, an antibody, a chemotherapeutic agent or a radioactive moiety, or an immunomodulatory drug (IMiD). Examples of useful secondary or adjunctive therapeutic agents that can be used with the cells of the invention include, but are not limited to: chemotherapeutic agents including alkylating agents such as thiotepa and cyclophaophamide, alkyl sulfonates such as busulfan, improsulfan and piposulfan; aziridines such as benzodopa, corboquone; ethyleneimines and methylamelamines including altreamine, triethylenemelamine, trietyelenephosphoramide; delta-9-tetrahydocannabinol; a camptothecin, irinotecan, acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB 1-TM1); eleutherobin; pancratistatin; a sarcodictyin; spongistatin; nitrogen mustards such as chlorambucil, chlornaphazine, cholophosphamide, estramustine, ifosfanide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gammall and calicheamicin omegall (see, e.g., Agnew, Chem. Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholinodoxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albuminengineered nanoparticle formulation of paclitaxel (ABRAXANET™), and doxetaxel (TAXOTERE®); chloranbucil; 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; cyclosporine, sirolimus, rapamycin, rapalogs, ibandronate; topoisomerase inhibitor RFS 2000; difluoromethylomithine (DMFO); retinoids such as retinoic acid; CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU, leucovovin; anti-estrogens and selective estrogen receptor modulators (SERMs), including, for example, tamoxifen (including NOLVADEX® tamoxifen), raloxifene (EVISTA®), droloxifene, 4-hydroxytamoxifen, trioxifene, keoxifene, LY1 17018, onapristone, and toremifene (FARESTON®); anti-progesterones; estrogen receptor down-regulators (ERDs); estrogen receptor antagonists such as fulvestrant (FASLODEX®); agents that function to suppress or shut down the ovaries, for example, leutinizing hormone-releasing hormone (LHRH) agonists such as leuprolide acetate (LUPRON® and ELIGARD®), goserelin acetate, buserelin acetate and tripterelin; other antiandrogens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, megestrol acetate (MEGASE®), exemestane (AROMASIN®), formestanie, fadrozole, vorozole (RIVISOR®), letrozole (FEMARA®), and anastrozole (ARIMIDEX®); bisphosphonates such as clodronate (for example, BONEFOS® or OST AC®), etidronate (DIDROCAL®), NE-58095, zoledronic acid/zoledronate (ZOMETA®), alendronate (FOSAMAX®), pamidronate (AREDIA®), tiludronate (SKELID®), or risedronate (ACTONEL®); troxacitabine (a 1,3-dioxolane nucleoside cytosine analog); aptamers, described for example in U.S. Pat. No. 6,344,321, which is herein incorporated by reference in its entirety; anti HGF monoclonal antibodies (e.g., AV299 from Aveo, AMG102, from Amgen); truncated mTOR variants (e.g., CGEN241 from Compugen); protein kinase inhibitors that block mTOR induced pathways (e.g., ARQ197 from Arqule, XL880 from Exelexis, SGX523 from SGX Pharmaceuticals, MP470 from Supergen, PF2341066 from Pfizer); vaccines such as THERATOPE® vaccine and gene therapy vaccines, for example, ALLOVECTIN® vaccine, LEUVECTIN® vaccine, and VAXID® vaccine; topoisomerase 1 inhibitor (e.g., LURTOTECAN®); rmRH (e.g., ABARELIX®); lapatinib ditosylate (an ErbB-2 and EGFR dual tyrosine kinase small molecule inhibitor also known as GW572016); COX-2 inhibitors such as celecoxib (CELEBREX®; 4-(5-(4-methylphenyl)-3-(trifluoromethyl)-1H-pyrazol-1-yl) benzenesulfonamide; and pharmaceutically acceptable salts, acids or derivatives of any of the above.

### EXAMPLES

### Example 1. Generating iPSCs Expressing an HSV-TK-PSMA Fusion

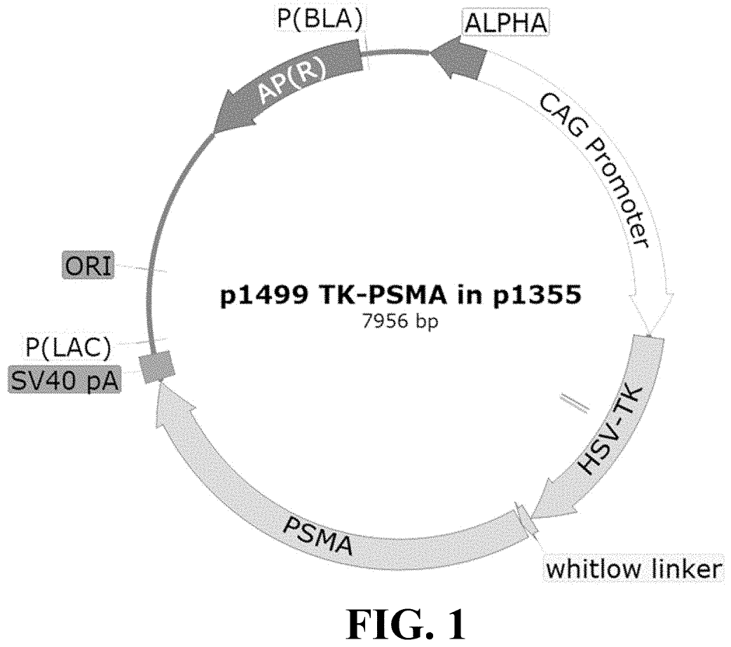

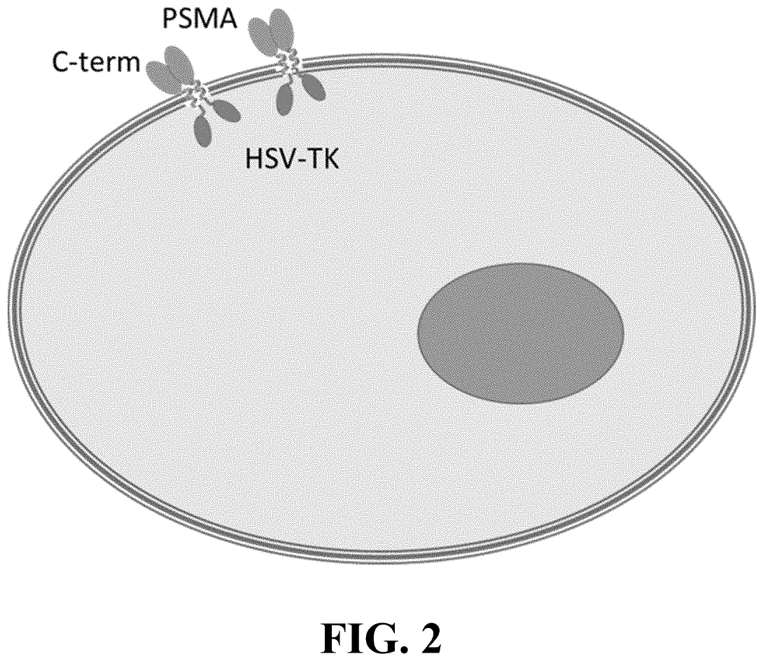

The HSV-TK-PSMA fusion comprises a herpes simplex virus thymidine kinase (HSV-tk) fused to a prostate-specific membrane antigen (PSMA) polypeptide via a Whitlow linker. The transgene sequence of SEQ ID NO: 74 was synthesized in 2 gBlocks from IDT, Inc. (Coralville, Iowa), and cloned by Infusion cloning (Takara, Inc; Shiga, Japan) into vector p1355 that possesses a CAG promoter and an SV40 terminator for strong expression in mammalian cells. The resulting plasmid (p1499; shown in FIG. 1) was sequenced and grown up for purification using the Qiagen maxi-prep kit (Qiagen, Inc; Hilden, Germany). Once transfected into cells, the fusion protein is expressed at the cell surface with the PSMA polypeptide on the extracellular membrane surface and the HSV-tk on the intracellular membrane surface (FIG. 2).

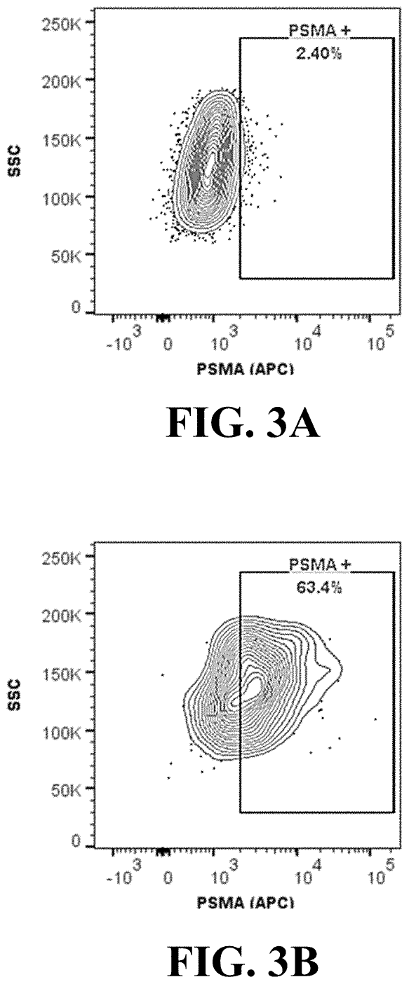

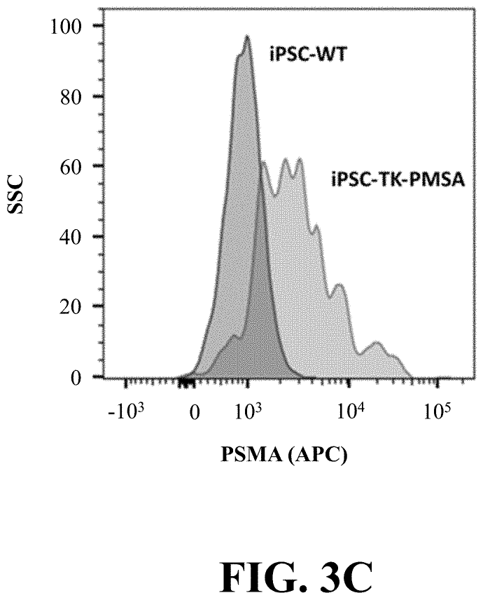

The purified plasmid was transfected into iPSC cells using Stemfectamine (Thermofisher, Inc.; Waltham, Mass.). Three days following transfection, transfected and untransfected cells (WT control) were stained with an APC labelled anti-PSMA antibody (cat #342507, Biolegend; San Diego, Calif.). Cells expressing PSMA were then quantified using flow cytometry. Results demonstrate that iPSCs transfected with HSV-TK-PSMA detectably expressed PSMA while untransfected cells did not (FIGS. 3A-C).

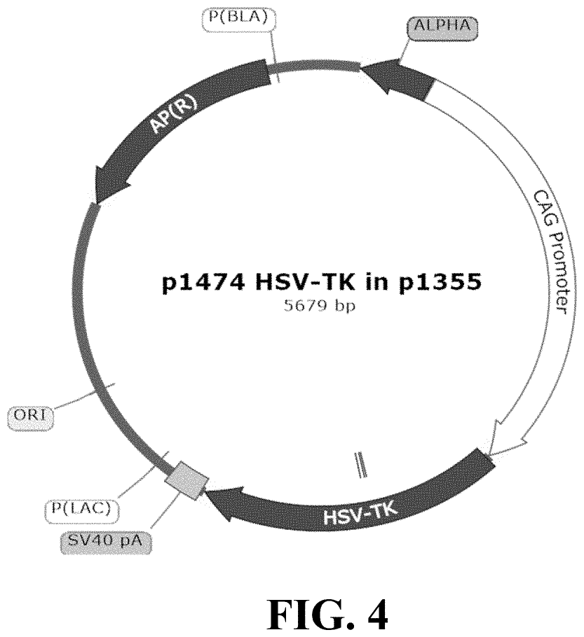

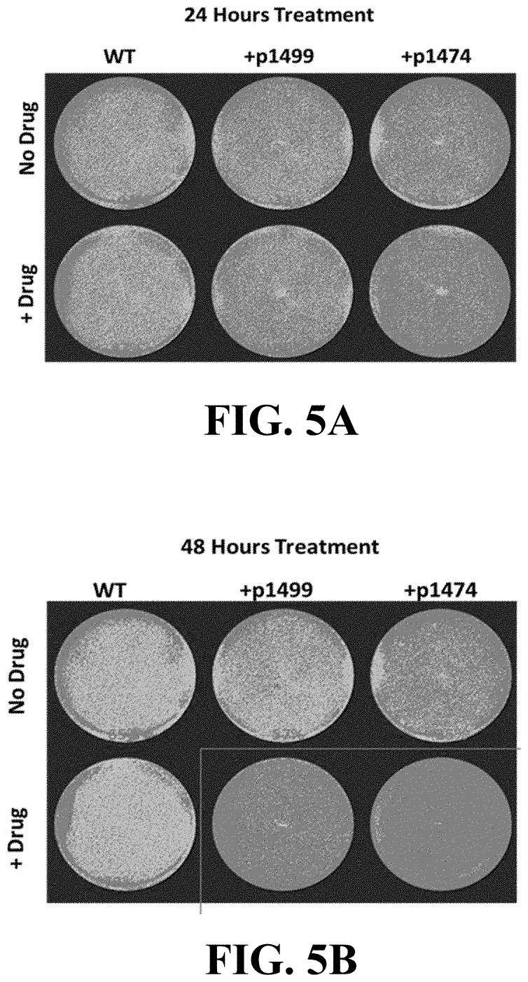

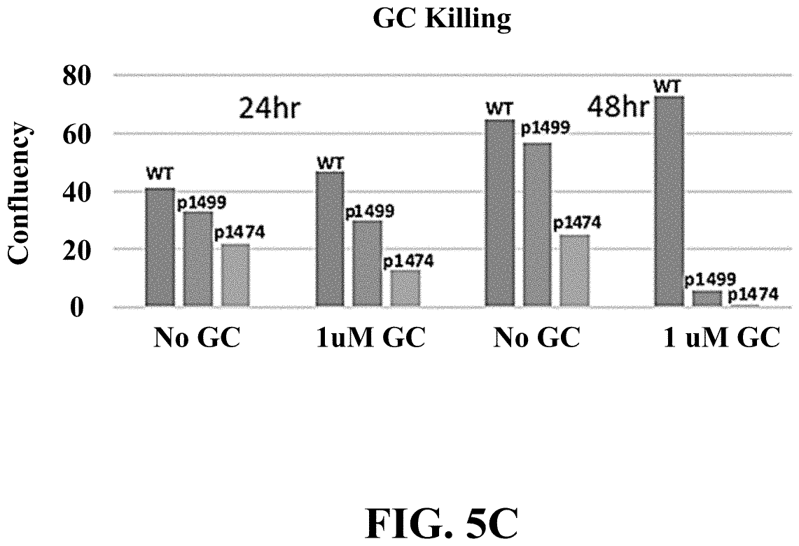

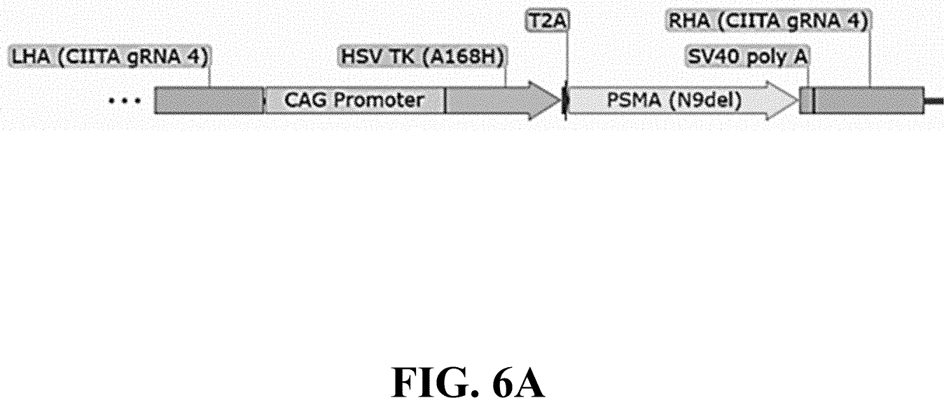

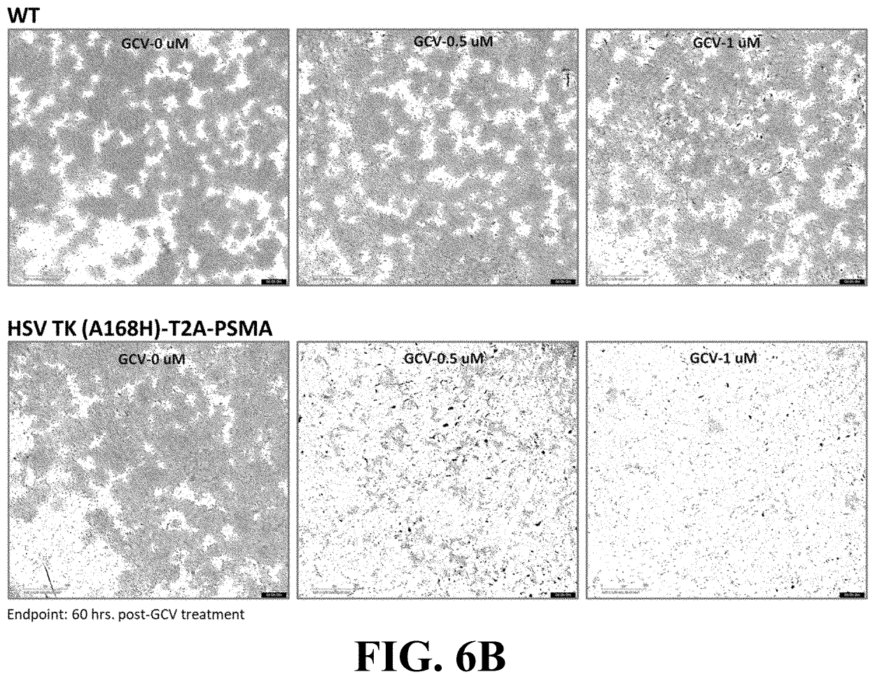

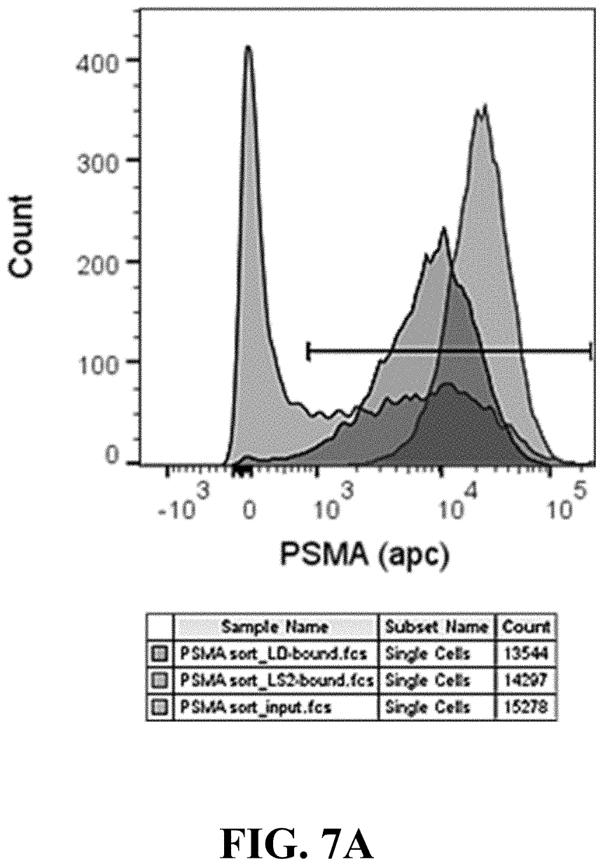

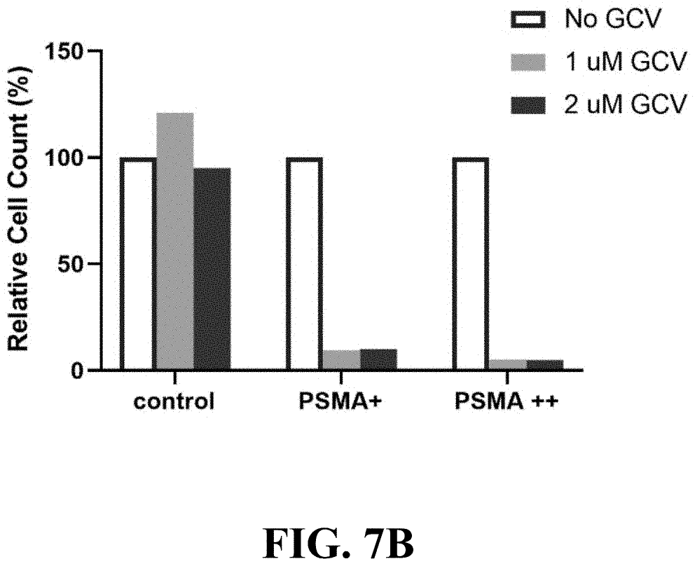

The ability to induce cell death in cells expressing the HSV-TK-PSMA fusion or HSV-TK alone was next tested. The WT HSV-TK coding sequence was synthesized and cloned into a vector p1355 that possesses a CAG promoter and an SV40 terminator. The resulting plasmid (p1474; shown in FIG. 4) was sequenced and grown up for purification using the Qiagen maxi-prep kit. Next, iPSCs were transfected with the p1499 or p1474 plasmids. Transfected and untransfected cells were plated into 6 well dishes (2×10⁵ cells/well) and treated with and without 1 μM ganciclovir and imaged at 24 and 48 hours to monitor cell growth. FIGS. 5A and 5B show that at 24 hours and 48 hours, respectively, ganciclovir treatment had no effect on the cell growth of untransfected iPSCs. However, iPSCs expressing the HSV-TK-PSMA fusion or HSV-TK alone had reduced confluency at 24 and 48 hours after ganciclovir treatment compared with untreated cells. iPSCs expressing the HSV-TK-PSMA and treated with ganciclovir had 6% confluency after 48 hours compared to the 73% cell confluency of WT cells treated with ganciclovir at the same timepoint (FIG. 5C). The data demonstrates that cells expressing HSV-TK-PSMA fusion can be effectively killed using ganciclovir treatment. It will be appreciated by those skilled in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the present description.

1. A polynucleotide encoding a combined artificial cell death/reporter system polypeptide comprising an intracellular domain having a herpes simplex virus thymidine kinase (HSV-tk) and a linker, a transmembrane region, and an extracellular domain comprising a prostate-specific membrane antigen (PSMA) extracellular domain or fragment thereof.

2. The polynucleotide according to claim 1, wherein the linker comprises or consists of an amino acid sequence selected from the group consisting of SEQ ID NO: 25 to 56, such as the linker consisting of the amino acid sequence of SEQ ID NO: 48.

3. The polynucleotide according to claim 1, wherein the linker comprises an autoprotease peptide sequence, such as an autoprotease peptide sequence selected from the group consisting of porcine teschovirus-1 2A (P2A), thosea asigna virus 2A (T2A), equine rhinitis A virus 2A (E2A), foot-and-mouth disease virus 18 2A (F2A).

4. The polynucleotide according to claim 3, wherein the autoprotease peptide is a thosea asigna virus 2A (T2A) peptide comprising the amino acid of SEQ ID NO: 75 or 87.

5. The polynucleotide according to claim 1, wherein the HSV-tk comprises an amino acid sequence having at least 90%, 91%, 92%, 93%, 94%, 95%, 96%, 97%, 98%, 99% or 100% sequence identity to SEQ ID NO: 71 or 89.

6. The polynucleotide according to claim 1, wherein the artificial cell death/reporter system polypeptide comprises the HSV-tk fused to a truncated variant PSMA polypeptide via the linker.

7. The polynucleotide according to claim 6, wherein the truncated variant PSMA polypeptide comprises an amino acid sequence having at least 90%, 91%, 92%, 93%, 94%, 95%, 96%, 97%, 98%, 99% or 100% sequence identity to SEQ ID NO: 72.

8. The polynucleotide according to claim 1, wherein the artificial cell death/reporter system polypeptide comprises an amino acid sequence having at least 90%, 91%, 92%, 93%, 94%, 95%, 96%, 97%, 98%, 99% or 100% sequence identity to SEQ ID NO: 73.

9. The polynucleotide according to claim 3, wherein the artificial cell death/reporter system polypeptide comprises an amino acid sequence having at least 90%, 91%, 92%, 93%, 94%, 95%, 96%, 97%, 98%, 99% or 100% sequence identity to a sequence selected from the group consisting of SEQ ID NOs: 76, 93, and 94.

10. The polynucleotide according to claim 1, comprising a polynucleotide sequence having at least 90%, 91%, 92%, 93%, 94%, 95%, 96%, 97%, 98%, 99% or 100% sequence identity to a sequence selected from the group consisting of SEQ ID NOs: 74, 77, and 95.

11. A polynucleotide encoding a combined artificial cell death/reporter system/CD24 polypeptide comprising a cluster of differentiation 24 (CD24) fused to a prostate-specific membrane antigen (PSMA) extracellular domain or fragment thereof via a peptide linker.

12. The polynucleotide according to claim 11, wherein the CD24 comprises an amino acid sequence having at least 90%, 91%, 92%, 93%, 94%, 95%, 96%, 97%, 98%, 99% or 100% sequence identity to SEQ ID NO: 78.

13. The polynucleotide according to claim 11, wherein the artificial cell death/reporter system/CD24 polypeptide comprises the CD24 fused to a truncated variant PSMA polypeptide via the linker.

14. The polynucleotide according to claim 13, wherein the truncated variant PSMA polypeptide comprises an amino acid sequence having at least 90%, 91%, 92%, 93%, 94%, 95%, 96%, 97%, 98%, 99% or 100% sequence identity to SEQ ID NO: 72.

15. The polynucleotide according to claim 11, wherein the linker comprises an autoprotease peptide sequence, such as an autoprotease peptide sequence selected from the group consisting of porcine teschovirus-1 2A (P2A), thosea asigna virus 2A (T2A), equine rhinitis A virus 2A (E2A), foot-and-mouth disease virus 18 2A (F2A).

16. The polynucleotide according to claim 150, wherein the autoprotease peptide is a thosea asigna virus 2A (T2A) peptide comprising the amino acid of SEQ ID NO: 75 or 87.

17. The polynucleotide according to claim 11, wherein the artificial cell death/reporter system/CD24 polypeptide comprises an amino acid sequence having at least 90%, 91%, 92%, 93%, 94%, 95%, 96%, 97%, 98%, 99% or 100% sequence identity to SEQ ID NO: 79.

18. The polynucleotide according to claim 11, comprising a polynucleotide sequence having at least 90%, 91%, 92%, 93%, 94%, 95%, 96%, 97%, 98%, 99% or 100% sequence identity to SEQ ID NO: 80.

19. A polynucleotide encoding a combined artificial cell death/reporter system/CD52 polypeptide comprising a cluster of differentiation 52 (CD52) fused to a herpes simplex virus thymidine kinase (HSV-tk) or fragment thereof via a peptide linker.

20. The polynucleotide according to claim 190, wherein the CD52 comprises an amino acid sequence having at least 90%, 91%, 92%, 93%, 94%, 95%, 96%, 97%, 98%, 99% or 100% sequence identity to SEQ ID NO: 91.

21. The polynucleotide according to claim 19, wherein the artificial cell death/reporter system/CD52 polypeptide comprises the CD52 fused to a truncated variant HSV-tk polypeptide via the linker.

22. The polynucleotide according to claim 19, wherein the linker comprises an autoprotease peptide sequence, such as an autoprotease peptide sequence selected from the group consisting of porcine teschovirus-1 2A (P2A), thosea asigna virus 2A (T2A), equine rhinitis A virus 2A (E2A), foot-and-mouth disease virus 18 2A (F2A).

23. The polynucleotide according to claim 22, wherein the autoprotease peptide is a thosea asigna virus 2A (T2A) peptide comprising the amino acid of SEQ ID NO: 75 or 87.

24. The polynucleotide according to claim 19 wherein the artificial cell death/reporter system/CD52 polypeptide comprises an amino acid sequence having at least 90%, 91%, 92%, 93%, 94%, 95%, 96%, 97%, 98%, 99% or 100% sequence identity to SEQ ID NO: 96 or 97.

25. The polynucleotide according to claim 190, comprising a polynucleotide sequence having at least 90%, 91%, 92%, 93%, 94%, 95%, 96%, 97%, 98%, 99% or 100% sequence identity to SEQ ID NO: 98.

26. The polynucleotide according to claim 19, wherein the HSV-tk comprises an amino acid sequence having at least 90%, 91%, 92%, 93%, 94%, 95%, 96%, 97%, 98%, 99% or 100% sequence identity to SEQ ID NO: 71 or 89.

27. An artificial cell death/reporter system polypeptide encoded by the polynucleotide according to claim 1.

28. A vector comprising the polynucleotide according to claim 1.

29. A cell, such as an immune cell, an induced pluripotent stem cell (iPSC) or derivative cell thereof comprising the polynucleotide according to claim 1, wherein the PSMA polypeptide is expressed extracellularly and the HSV-tk is expressed intracellularly.

30. (canceled)

31. A method of eliminating the cell according to claim 29, comprising contacting the cell with one or more agents that bind to the artificial cell death polypeptide to thereby induce death of the cell.

32.-33. (canceled)

34. A method of producing a cell expressing the combined artificial cell death/reporter system polypeptide, comprising introducing the polynucleotide according to claim 1 into a cell to thereby produce the cell expressing the combined artificial cell death/reporter system polypeptide.


SEQUENCE LISTING (98 sequences)

SEQ ID NO: 1 - protein, 22 aa, Homo sapiens
MLLLVTSLLLCELPHPAFLLIP

SEQ ID NO: 2 - protein, 120 aa, Artificial Sequence
  FMC63 VH
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSS

SEQ ID NO: 3 - protein, 18 aa, Artificial Sequence
  Whitlow Linker
GSTSGSGKPGSGEGSTKG

SEQ ID NO: 4 - protein, 107 aa, Artificial Sequence
  FMC63 VL
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 5 - protein, 107 aa, Homo sapiens
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 6 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - protein, 245 aa, Artificial Sequence
  FMC63 scFV
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSGSTSGSGKPGSGEGSTKGDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 8 - protein, 42 aa, Homo sapiens
KRGRKKLLYIFKQPFMRPVQTTQEEDGCSCRFPEEEEGGCEL

SEQ ID NO: 9 - protein, 94 aa, Homo sapiens
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 10 - protein, 62 aa, Homo sapiens
KKVAKKPTNKAPHPKQEPQEINFPDDLPGSNTAAPVQETLHGCQPVTQEDGKESRISVQERQ

SEQ ID NO: 11 - protein, 42 aa, Homo sapiens
ALYLLRRDQRLPPDAHKPPGGGSFRTPIQEEQADAHSTLAKI

SEQ ID NO: 12 - protein, 25 aa, Homo sapiens
TYCFAPRCRERRRNERLRRESVRPV

SEQ ID NO: 13 - protein, 61 aa, Homo sapiens
KWKKKKRPRNSYKCGTNTMEREESEQTKKREKIHIPERSDEAQRVFKSSKTSSCDKSDTCF

SEQ ID NO: 14 - protein, 48 aa, Homo sapiens
QRRKYRSNKGESPVEPAEPCHYSCPREEEGSTIPIQEDYRKPEPACSP

SEQ ID NO: 15 - protein, 38 aa, Homo sapiens
CWLTKKKYSSSVHDPNGEYMFMRAVNTAKKSRLTDVTL

SEQ ID NO: 16 - protein, 51 aa, Homo sapiens
MGWIRGRRSRHSWEMSEFHNYNLDLKKSDFSTRWQKQRCPVVKSKCRENAS

SEQ ID NO: 17 - protein, 24 aa, Homo sapiens
LCARPRRSPAQEDGKVYINMPGRG

SEQ ID NO: 18 - protein, 52 aa, Homo sapiens
YFLGRLVPRGRGAAEAATRKQRITETESPYQELQGQRSDVYSDLNTQRPYYK

SEQ ID NO: 19 - protein, 120 aa, Homo sapiens
WRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 20 - protein, 41 aa, Homo sapiens
RSKRSRLLHSDYMNMTPRRPGPTRKHYQPYAPPRDFAAYRS

SEQ ID NO: 21 - protein, 47 aa, Homo sapiens
TTTPAPRPPTPAPTIASQPLSLRPEACRPAAGGAVHTRGLDFACDIY

SEQ ID NO: 22 - protein, 39 aa, Homo sapiens
IEVMYPPPYLDNEKSNGTIIHVKGKHLCPSPLFPGPSKP

SEQ ID NO: 23 - protein, 21 aa, Homo sapiens
IYIWAPLAGTCGVLLLSLVIT

SEQ ID NO: 24 - protein, 27 aa, Homo sapiens
FWVLVVVGGVLACYSLLVTVAFIIFWV

SEQ ID NO: 25 - protein, 15 aa, Artificial Sequence
  (G4S)3
GGGGSGGGGSGGGGS

SEQ ID NO: 26 - protein, 20 aa, Artificial Sequence
  Linker 3
GGSEGKSSGSGSESKSTGGS

SEQ ID NO: 27 - protein, 8 aa, Artificial Sequence
  Linker 4
GGGSGGGS

SEQ ID NO: 28 - protein, 12 aa, Artificial Sequence
  Linker 5
GGGSGGGSGGGS

SEQ ID NO: 29 - protein, 16 aa, Artificial Sequence
  Linker 6
GGGSGGGSGGGSGGGS

SEQ ID NO: 30 - protein, 20 aa, Artificial Sequence
  Linker 7
GGGSGGGSGGGSGGGSGGGS

SEQ ID NO: 31 - protein, 20 aa, Artificial Sequence
  Linker 8
GGGGSGGGGSGGGGSGGGGS

SEQ ID NO: 32 - protein, 25 aa, Artificial Sequence
  Linker 9
GGGGSGGGGSGGGGSGGGGSGGGGS

SEQ ID NO: 33 - protein, 14 aa, Artificial Sequence
  Linker 10
IRPRAIGGSKPRVA

SEQ ID NO: 34 - protein, 15 aa, Artificial Sequence
  Linker 11
GKGGSGKGGSGKGGS

SEQ ID NO: 35 - protein, 15 aa, Artificial Sequence
  Linker 12
GGKGSGGKGSGGKGS

SEQ ID NO: 36 - protein, 15 aa, Artificial Sequence
  Linker 13
GGGKSGGGKSGGGKS

SEQ ID NO: 37 - protein, 15 aa, Artificial Sequence
  Linker 14
GKGKSGKGKSGKGKS

SEQ ID NO: 38 - protein, 15 aa, Artificial Sequence
  Linker 15
GGGKSGGKGSGKGGS

SEQ ID NO: 39 - protein, 15 aa, Artificial Sequence
  Linker 16
GKPGSGKPGSGKPGS

SEQ ID NO: 40 - protein, 20 aa, Artificial Sequence
  Linker 17
GKPGSGKPGSGKPGSGKPGS

SEQ ID NO: 41 - protein, 20 aa, Artificial Sequence
  Linker 18
GKGKSGKGKSGKGKSGKGKS

SEQ ID NO: 42 - protein, 14 aa, Artificial Sequence
  Linker 19
STAGDTHLGGEDFD

SEQ ID NO: 43 - protein, 15 aa, Artificial Sequence
  Linker 20
GEGGSGEGGSGEGGS

SEQ ID NO: 44 - protein, 15 aa, Artificial Sequence
  Linker 21
GGEGSGGEGSGGEGS

SEQ ID NO: 45 - protein, 15 aa, Artificial Sequence
  Linker 22
GEGESGEGESGEGES

SEQ ID NO: 46 - protein, 15 aa, Artificial Sequence
  Linker 23
GGGESGGEGSGEGGS

SEQ ID NO: 47 - protein, 20 aa, Artificial Sequence
  Linker 24
GEGESGEGESGEGESGEGES

SEQ ID NO: 48 - protein, 18 aa, Artificial Sequence
  Linker 25
GSTSGSGKPGSGEGSTKG

SEQ ID NO: 49 - protein, 19 aa, Artificial Sequence
  Linker 26
PRGASKSGSASQTGSAPGS

SEQ ID NO: 50 - protein, 19 aa, Artificial Sequence
  Linker 27
GTAAAGAGAAGGAAAGAAG

SEQ ID NO: 51 - protein, 19 aa, Artificial Sequence
  Linker 28
GTSGSSGSGSGGSGSGGGG

SEQ ID NO: 52 - protein, 20 aa, Artificial Sequence
  Linker 29
GKPGSGKPGSGKPGSGKPGS

SEQ ID NO: 53 - protein, 4 aa, Artificial Sequence
  Linker 30
GSGS

SEQ ID NO: 54 - protein, 10 aa, Artificial Sequence
  Linker 31
APAPAPAPAP

SEQ ID NO: 55 - protein, 20 aa, Artificial Sequence
  Linker 32
APAPAPAPAPAPAPAPAPAP

SEQ ID NO: 56 - protein, 32 aa, Artificial Sequence
  Linker 33
AEAAAKEAAAKEAAAAKEAAAAKEAAAAKAAA

SEQ ID NO: 57 - protein, 15 aa, Homo sapiens
EPKSCDKTHTCPPCP

SEQ ID NO: 58 - protein, 12 aa, Homo sapiens
ERKCCVECPPCP

SEQ ID NO: 59 - protein, 62 aa, Homo sapiens
ELKTPLGDTTHTCPRCPEPKSCDTPPPCPRCPEPKSCDTPPPCPRCPEPKSCDTPPPCPRCP

SEQ ID NO: 60 - protein, 12 aa, Homo sapiens
ESKYGPPCPSCP

SEQ ID NO: 61 - protein, 486 aa, Artificial Sequence
  anti-CD19 scFv CAR
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 62 - DNA, 1461 bp, Artificial Sequence
  anti-CD19 scFv CAR
atgctgctgctggtcacatctctgctgctgtgcgagctgccccatcctgcctttctgctgatccccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcatcgaagtgatgtaccctccaccttacctggacaacgagaagtccaacggcaccatcatccacgtgaagggcaagcacctgtgtccttctccactgttccccggacctagcaagcctttctgggtgctcgttgttgttggcggcgtgctggcctgttacagcctgctggttaccgtggccttcatcatcttttgggtccgaagcaagcggagccggctgctgcactccgactacatgaacatgacccctagacggcccggaccaaccagaaagcactaccagccttacgctcctcctagagacttcgccgcctaccggtccagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggactgagcaccgccaccaaggatacctatgacgccctgcacatgcaggccctgcctccaagatga

SEQ ID NO: 63 - DNA, 1724 bp, Artificial Sequence
  CAG Promoter
attgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaa

SEQ ID NO: 64 - DNA, 121 bp, Artificial Sequence
  SV40 terminator/polyadenylation
aacttgtttattgcagcttataatggttacaaataaagcaatagcatcacaaatttcacaaataaagcatttttttcactgcattctagttgtggtttgtccaaactcatcaatgtatctt

SEQ ID NO: 65 - protein, 335 aa, Homo sapiens
HSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 66 - protein, 504 aa, Artificial Sequence
  HLA-G signal peptide-B2M-HLA-E
MVVMAPRTLFLLLSGALTLTETWAVMAPRTLILGGGGSGGGGSGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWKPASQPTIPIVGIIAGLVLLGSVVSGAVVAAVIWRKKSSGGKGGSYSKAEWSDSAQGSESHSL

SEQ ID NO: 67 - DNA, 1515 bp, Artificial Sequence
  HLA-G signal peptide-B2M-HLA-E
atggtggtcatggcccctagaacactgttcctgctgctgtctggcgccctgacactgacagagacatgggccgtgatggcccccagaaccctgatcctgggcggcggtggttcaggcggaggaggttcaggaggagggggtagtggaggtggtggttctatccagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgacactgagccagcctaagatcgtgaagtgggatcgcgatatgggcggaggcggatctggtggcggaggaagtggcggcggaggatctggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaa

SEQ ID NO: 68 - protein, 312 aa, Homo sapiens
HSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 69 - protein, 471 aa, Artificial Sequence
  HLA-G signal peptide-B2M-HLA-G
MVVMAPRTLFLLLSGALTLTETWARIIPRHLQLGGGGSGGGGSIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWKQSSLPTIPIMGIVAGLVVLAAVVTGAAVAAVLWRKKSSD

SEQ ID NO: 70 - DNA, 1422 bp, Artificial Sequence
  HLA-G signal peptide-B2M-HLA-G
gccaccatggtggtcatggcgccccgaaccctcttcctgctgctctcgggggccctgaccctgaccgagacctgggcgcggatcattccccgacatctgcaactgggaggcggcggttcaggagggggcggatcgatccaacgcacccccaagatccaggtctactccagacacccggccgaaaacggaaagtcgaacttcctgaactgctatgtgtcaggattccacccgtccgacatcgaggtggacctcctgaagaacggcgaacgcattgagaaggtcgagcactccgatctgtcgttctccaaggactggtccttctaccttctctactataccgaattcaccccgaccgagaaggacgaatacgcctgccgggtcaaccacgtgaccctgagccagccaaagatcgtgaaatgggaccgcgatatgggaggaggaggttccggcggaggaggaagcggaggcggaggttccggctcccactccatgaggtatttcagcgccgccgtgtcccggcctggccgcggagagcctcgcttcatcgccatgggatacgtggacgacacccagttcgtcagattcgacagcgacagcgcctgtcctcggatggaacctagagcaccttgggtcgagcaagagggccctgagtactgggaagaagagacacggaacaccaaggctcacgcccagaccgacagaatgaacctgcagaccctgcggggctactacaatcagtctgaggccagcagccatactctgcagtggatgatcggctgcgatctgggctctgatggcagactgctgagaggctacgagcagtacgcctacgacggcaaggattatctggccctgaacgaggacctgcggtcttggacagctgccgatacagccgctcagatcagcaagagaaagtgcgaggccgccaatgtggccgaacagagaagggcttacctggaaggcacctgtgtggaatggctgcacagatacctggaaaacggcaaagagatgctgcagcgggccgatcctcctaagacacatgtgacccaccatcctgtgttcgactacgaggccacactgagatgttgggccctgggcttttaccctgccgagatcatcctgacctggcagcgagatggcgaggatcagacccaggatgtggaactggtggaaaccagacctgccggcgacggcacctttcagaaatgggctgctgtggtggtgcccagcggagaggaacagagatacacctgtcacgtgcagcacgagggactgcctgaacctctgatgctgagatggaagcagagcagcctgcctacaatccccatcatgggaatcgtggccggactggtggttctggccgctgttgttacaggtgctgcagtggctgccgtgctgtggcggaagaaaagcagcgactga

SEQ ID NO: 71 - protein, 376 aa, Artificial Sequence
  HSV thymidine kinase
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 72 - protein, 741 aa, Artificial Sequence
  PSMA variant
MWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 73 - protein, 1135 aa, Artificial Sequence
  HSV-TK-PSMA fusion
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSTSGSGKPGSGEGSTKGMWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 74 - DNA, 3408 bp, Artificial Sequence
  HSV-TK-PSMA fusion
atggcctcatatccctgccaccagcatgcgagtgcgtttgatcaagctgcacggtcaagaggacattccaataggcgaactgcactgagacctaggcgacaacaagaagcaacggaggtccgccttgagcagaagatgcctactcttcttagggtttacatcgatggtccgcacggaatgggtaagaccacgactactcaactgcttgtcgcattgggctccagggatgacatagtttacgttccggaacctatgacttattggcaggtgttgggggcatccgaaaccatagctaatatttataccacgcaacaccgcttggaccaaggagagatctctgcgggagatgcagcggtggtaatgaccagcgcacaaatcaccatggggatgccgtatgccgtaacagacgcggtgctggccccccatatcggtggtgaagccggttcaagtcatgcgcctccgccggcgctgacattgatctttgatcggcatcctattgcggcactcctgtgttatccagccgcacgctatctgatgggaagcatgaccccacaagcggttctggcctttgtggcattgatcccaccaacattgcctggaactaatattgtactgggcgctctccccgaggatcgacatattgataggctcgcgaaacgccaacgccctggtgaaaggcttgatttggccatgctcgcggctatacgccgcgtctatgggttgctcgccaatactgtgagatacctgcagggcggggggtcatggcgcgaggattggggtcagttgtcaggcacggcagtgcccccacagggggcggagcctcagtcaaatgcaggcccaagaccgcatattggtgatactctcttcacgttgtttcgggcgccagaattgcttgcccccaacggtgatctttataacgtcttcgcatgggccctcgatgtcttggccaagaggctgcgaccgatgcacgtttttattctcgactatgaccagtcacctgcgggatgcagggatgcgttgctccagttgacaagtggcatggtgcagacacatgtgacgacacccggatcaatacctacgatatgtgatctcgcaaggacctttgcgagggaaatgggggaagctaatggctcaacttctggatccggaaagcccggtagcggcgaaggttctacaaagggtatgtggaacctgttggcaagacgcccccgctggctctgtgccggtgctctggtactggcggggggattttttctcttgggatttctttttgggtggttcataaaaagttctaatgaggccacaaatatcactccgaaacacaatatgaaggcatttctggacgagctcaaagcggaaaatattaagaaattcctgtataactttactcagatacctcatctggctggcaccgagcaaaacttccagttggctaagcagattcaatcacagtggaaagagttcggtctcgatagcgttgaattggcacactacgatgtccttcttagctatcctaataaaacacatccgaactacataagtatcattaatgaggacggcaacgaaattttcaacacctctctttttgaacctcctccgccgggatatgaaaacgtgagcgatatcgttccccccttttccgcgttctcaccacagggaatgccggagggtgaccttgtttatgtaaattatgccagaacggaagatttctttaaacttgaacgcgatatgaaaatcaactgctctggtaagattgttattgcccgatacgggaaggtattcaggggtaacaaagtgaaaaacgctcagctggcgggtgccaaaggagtgatcctttattctgaccctgcggattatttcgcaccaggcgtaaaatcatatcctgacggttggaaccttcctggaggtggagtacagcgcggaaatatattgaatcttaacggagccggtgacccactgactcctggataccccgcaaacgagtatgcctatcgacgcggcattgccgaagcggtgggactgccctcaatacctgtacatcctattggatactatgatgctcagaaacttttggagaagatgggtggaagtgccccgcctgatagttcctggagaggctcccttaaggttccatataacgtaggtccagggtttacgggcaacttttcaacacaaaaggtaaagatgcatatacattcaactaacgaggtgacgaggatatacaatgtaatcggaactctgaggggagccgtagagcctgatcgatatgtcatcttggggggccacagggatagttgggtctttggtggaattgatcctcagtcaggtgcggctgtagtccacgagattgtccgctctttcggcacgctgaagaaggaggggtggagaccccgaaggactattttgtttgcctcttgggatgctgaagaatttggtctgctcggatcaacggagtgggctgaagagaattctaggttgttgcaagaacgcggtgtggcctacataaatgcggacagtagtatagaaggcaattacacacttcgagtggattgcaccccgcttatgtacagtctggtacataacctgacgaaggagcttaaatcacctgatgaaggattcgaggggaaatccctttacgaatcatggactaaaaagtcaccttcccctgaatttagtgggatgccgcgcataagtaaactcgggtccggaaacgacttcgaagttttcttccaacgattgggtatcgcctctggacgagcacggtacaccaaaaattgggaaacgaacaaattttccggatatcctctctaccactctgtctatgaaacctacgagctggtggaaaagttttacgatccgatgtttaagtaccatttgaccgtcgcccaggtgcggggaggaatggtctttgaattggcaaatagtatagtccttccttttgattgtcgagattatgccgtcgtccttaggaagtatgctgacaagatttattcaatatctatgaagcacccccaagaaatgaagacctactcagtgtcctttgactccctgttctccgctgtgaagaacttcactgagatcgcctctaagttctcagagcgactgcaagattttgacaagagtaaccccattgttcttaggatgatgaatgaccagctcatgtttttggagagagcatttattgatccgctgggccttcccgaccggccattttacagacacgttatctatgctccttcaagtcacaataaatatgcaggagaatcctttcctgggatctacgatgccctcttcgacatagaaagtaaggttgatccctccaaggcgtggggggaagttaaacgacagatatatgtcgctgcattcaccgtacaggccgccgcagagacacttagtgaggttgcgtga

SEQ ID NO: 75 - protein, 22 aa, Artificial Sequence
  T2A
SGSGEGRGSLLTCGDVEENPGP

SEQ ID NO: 76 - protein, 1139 aa, Artificial Sequence
  HSV-TK-T2A-PSMA
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANSGSGEGRGSLLTCGDVEENPGPMWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 77 - DNA, 3420 bp, Artificial Sequence
  HSV-TK-T2A-PSMA
atggcaagttacccttgccaccagcatgctagcgctttcgatcaagcggcccgcagtcggggccatagcaatagaaggaccgcattgcgcccgcgccgacagcaggaagctaccgaagtacgactggaacaaaaaatgcccactcttctgagagtatacatcgatgggccacacggcatgggcaaaacgacgactacccaactcttggtagctcttggtagccgggatgatatcgtatatgtgccagaacctatgacctattggcaggtcctcggcgccagtgaaaccatcgccaacatatatacgacgcaacataggcttgaccagggtgaaatctccgcaggtgacgcggcagtggtcatgactagcgcccaaatcacgatgggaatgccttatgcggtgactgacgcagtactcgctcctcacattggaggtgaagcggggagctcccacgcaccgccgcccgctcttacgctcattttcgatcgccaccctatagctgccctgctctgttatcccgcggccaggtacttgatggggtccatgaccccccaggcggtgctggccttcgttgcgttgataccgccaactctccccggcactaatattgttctcggagcacttccagaagacaggcatattgacaggttggctaagcgccaaaggcccggtgaacgactcgacctggctatgcttgctgccatccgccgcgtctatgggctgctggctaatacggtcaggtatcttcaaggcggcggatcttggagggaagattggggacagctcagtggaacggctgtacctccacaaggggccgaacctcagtcaaatgcaggtcctcgccctcacattggagatacactttttactcttttccgggctccagaactgctggcaccaaatggcgacctgtacaatgtttttgcctgggctctggatgttttggcaaaaaggcttcggccgatgcacgtatttattttggactatgatcagtcaccggctggttgtagagacgcattgcttcaacttacatccgggatggtacaaacgcatgtaacaaccccagggtcaattccaactatctgcgatctcgcccgcacattcgcaagagaaatgggtgaggctaactctggcagtggtgaaggccgcggatctctcctgacttgtggggatgtcgaagaaaacccgggacccatgtggaacctgttggcaagacgcccccgctggctctgtgccggtgctctggtactggcggggggattttttctcttgggatttctttttgggtggttcataaaaagttctaatgaggccacaaatatcactccgaaacacaatatgaaggcatttctggacgagctcaaagcggaaaatattaagaaattcctgtataactttactcagatacctcatctggctggcaccgagcaaaacttccagttggctaagcagattcaatcacagtggaaagagttcggtctcgatagcgttgaattggcacactacgatgtccttcttagctatcctaataaaacacatccgaactacataagtatcattaatgaggacggcaacgaaattttcaacacctctctttttgaacctcctccgccgggatatgaaaacgtgagcgatatcgttccccccttttccgcgttctcaccacagggaatgccggagggtgaccttgtttatgtaaattatgccagaacggaagatttctttaaacttgaacgcgatatgaaaatcaactgctctggtaagattgttattgcccgatacgggaaggtattcaggggtaacaaagtgaaaaacgctcagctggcgggtgccaaaggagtgatcctttattctgaccctgcggattatttcgcaccaggcgtaaaatcatatcctgacggttggaaccttcctggaggtggagtacagcgcggaaatatattgaatcttaacggagccggtgacccactgactcctggataccccgcaaacgagtatgcctatcgacgcggcattgccgaagcggtgggactgccctcaatacctgtacatcctattggatactatgatgctcagaaacttttggagaagatgggtggaagtgccccgcctgatagttcctggagaggctcccttaaggttccatataacgtaggtccagggtttacgggcaacttttcaacacaaaaggtaaagatgcatatacattcaactaacgaggtgacgaggatatacaatgtaatcggaactctgaggggagccgtagagcctgatcgatatgtcatcttggggggccacagggatagttgggtctttggtggaattgatcctcagtcaggtgcggctgtagtccacgagattgtccgctctttcggcacgctgaagaaggaggggtggagaccccgaaggactattttgtttgcctcttgggatgctgaagaatttggtctgctcggatcaacggagtgggctgaagagaattctaggttgttgcaagaacgcggtgtggcctacataaatgcggacagtagtatagaaggcaattacacacttcgagtggattgcaccccgcttatgtacagtctggtacataacctgacgaaggagcttaaatcacctgatgaaggattcgaggggaaatccctttacgaatcatggactaaaaagtcaccttcccctgaatttagtgggatgccgcgcataagtaaactcgggtccggaaacgacttcgaagttttcttccaacgattgggtatcgcctctggacgagcacggtacaccaaaaattgggaaacgaacaaattttccggatatcctctctaccactctgtctatgaaacctacgagctggtggaaaagttttacgatccgatgtttaagtaccatttgaccgtcgcccaggtgcggggaggaatggtctttgaattggcaaatagtatagtccttccttttgattgtcgagattatgccgtcgtccttaggaagtatgctgacaagatttattcaatatctatgaagcacccccaagaaatgaagacctactcagtgtcctttgactccctgttctccgctgtgaagaacttcactgagatcgcctctaagttctcagagcgactgcaagattttgacaagagtaaccccattgttcttaggatgatgaatgaccagctcatgtttttggagagagcatttattgatccgctgggccttcccgaccggccattttacagacacgttatctatgctccttcaagtcacaataaatatgcaggagaatcctttcctgggatctacgatgccctcttcgacatagaaagtaaggttgatccctccaaggcgtggggggaagttaaacgacagatatatgtcgctgcattcaccgtacaggccgccgcagagacacttagtgaggttgcgtga

SEQ ID NO: 78 - protein, 80 aa, Homo sapiens
MGRAMVARLGLGLLLLALLLPTQIYSSETTTGTSSNSSQSTSNSGLAPNPTNATTKAAGGALQSTASLFVVSLSLLHLYS

SEQ ID NO: 79 - protein, 843 aa, Artificial Sequence
  PSMA-T2A-CD24
MWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVASGSGEGRGSLLTCGDVEENPGPMGRAMVARLGLGLLLLALLLPTQIYSSETTTGTSSNSSQSTSNSGLAPNPTNATTKAAGGALQSTASLFVVSLSLLHLYS

SEQ ID NO: 80 - DNA, 2529 bp, Artificial Sequence
  PSMA-T2A-CD24
atgtggaacctgcttgctagaaggcctagatggctttgtgctggcgctctggttctggctggcggcttttttctgctgggcttcctgttcggctggttcatcaagagcagcaacgaggccaccaatatcacccctaagcacaacatgaaggcctttctggacgagctgaaggccgagaatatcaagaagttcctgtacaacttcacgcagatccctcacctggccggcaccgagcagaattttcagctggccaagcagatccagagccagtggaaagagttcggcctggactctgtggaactggcccactacgatgtgctgctgagctaccccaacaagacacaccccaactacatcagcatcatcaacgaggacggcaacgagatcttcaataccagcctgttcgagcctcctccacctggctacgagaacgtgtccgatatcgtgcctccattcagcgctttcagccctcaagggatgcctgagggcgatctggtgtacgtgaactacgccagaaccgaggacttcttcaagctggaacgggacatgaagatcaactgcagcggcaagatcgtgatcgccagatacggcaaggtgttccggggcaacaaagtgaagaacgcccaactggcaggcgccaagggcgtgatcctgtattctgatcccgccgactacttcgcccctggcgtgaagtcttatcccgacggctggaatcttcctggcggcggagttcagaggggcaatatcctgaacctgaacggcgctggcgaccctctgacacctggatatcctgccaacgagtacgcctacagacggggaattgccgaagctgtgggcctgccttctatccctgtgcacccaatcggctactacgacgcccagaaactgctggaaaagatgggcggaagcgcccctcctgactcttcttggagaggctctctgaaggtgccctacaatgtcggccctggcttcaccggcaacttcagcacccagaaagtgaaaatgcacatccacagcaccaacgaagtgacccggatctacaacgtgatcggcacactgagaggcgccgtggaacccgacagatatgtgatcctcggcggccacagagacagctgggtgttcggaggaatcgaccctcaatctggcgccgctgtggtgcacgaaatcgtgcggtcttttggcaccctgaagaaagaaggatggcgccccagacggaccatcctgtttgcctcttgggacgccgaggaatttggcctgctgggatctacagagtgggccgaagagaacagcagactgctgcaagaaagaggcgtggcctacatcaatgccgacagcagcatcgagggcaactacaccctgagagtggactgcacccctctgatgtactccctggtgcacaacctgaccaaagagctgaagtcccctgacgagggctttgagggcaagagcctgtacgagtcctggaccaagaagtccccatctcctgagttcagcggcatgcccagaatcagcaagctcggctccggcaatgacttcgaggtgttcttccagcggctgggaatcgcttctggcagagccagatacaccaagaactgggagacaaacaagttctccggctatcccctgtaccacagcgtgtacgagacatacgagctggtggaaaagttctacgaccccatgttcaagtaccacctgacagtggcccaagtgcgcggaggcatggtgttcgaactggccaatagcatcgtgctgcccttcgactgcagagactatgccgtggtgctgcggaagtacgccgataagatctacagcatctccatgaagcacccgcaagagatgaagacctacagcgtgtccttcgactccctgttctctgccgtgaagaacttcaccgagatcgccagcaagttcagcgagcggctgcaggacttcgacaagagcaaccctatcgtgctgaggatgatgaacgaccagctgatgttcctggaacgcgccttcatcgacccactgggactgcccgatagacccttctaccggcacgtgatctatgcccctagcagccacaacaaatacgccggcgagagcttccccggcatctacgatgccctgttcgacatcgagagcaaggtggacccaagcaaggcctggggagaagtgaagcggcagatctacgtggccgcattcacagtgcaggccgctgccgaaacactgtctgaggtggcctctggcagtggtgaaggccgcggatctctcctgacttgtggggatgtcgaagaaaacccgggacccatgggaagggcaatggttgcgcgactcggcttgggcttgcttttgctggctcttctcctcccaacacaaatttactcatccgaaacgaccacgggaacttcatctaattctagtcagtcaacatcaaattccgggcttgcacccaatcctacgaacgctaccacgaaggcggcgggaggcgctctccaatccaccgcctcacttttcgtggtttctctctcacttcttcatctctattct

SEQ ID NO: 81 - DNA, 1042 bp, Artificial Sequence
  B2M targeting left homology arm
gcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtc

SEQ ID NO: 82 - DNA, 1023 bp, Artificial Sequence
  B2M targeting right homology arm
atccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcag

SEQ ID NO: 83 - DNA, 7813 bp, Artificial Sequence
  B2M Targeting Plasmid
gcatataaaacctcagcagaaataaagaggttttgttgtttggtaagaacataccttgggttggttgggcacggtggctcgtgcctgtaatcccaacactttgggaggccaaggcaggctgatcacttgaagttgggagttcaagaccagcctggccaacatggtgaaatcccgtctctactgaaaatacaaaaattaaccaggcatggtggtgtgtgcctgtagtcccaggaatcacttgaacccaggaggcggaggttgcagtgagctgagatctcaccactgcacactgcactccagcctgggcaatggaatgagattccatcccaaaaaataaaaaaataaaaaaataaagaacataccttgggttgatccacttaggaacctcagataataacatctgccacgtatagagcaattgctatgtcccaggcactctactagacacttcatacagtttagaaaatcagatgggtgtagatcaaggcaggagcaggaaccaaaaagaaaggcataaacataagaaaaaaaatggaaggggtggaaacagagtacaataacatgagtaatttgatgggggctattatgaactgagaaatgaactttgaaaagtatcttggggccaaatcatgtagactcttgagtgatgtgttaaggaatgctatgagtgctgagagggcatcagaagtccttgagagcctccagagaaaggctcttaaaaatgcagcgcaatctccagtgacagaagatactgctagaaatctgctagaaaaaaaacaaaaaaggcatgtatagaggaattatgagggaaagataccaagtcacggtttattcttcaaaatggaggtggcttgttgggaaggtggaagctcatttggccagagtggaaatggaattgggagaaatcgatgaccaaatgtaaacacttggtgcctgatatagcttgacaccaagttagccccaagtgaaataccctggcaatattaatgtgtcttttcccgatattcctcaggtactccaaagattcaggtttactcacgtcggcctccaacgcgtagatctattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaagtcgacgccaccatggtggtcatggcccctagaacactgttcctgctgctgtctggcgccctgacactgacagagacatgggccgtgatggcccccagaaccctgatcctgggcggcggtggttcaggcggaggaggttcaggaggagggggtagtggaggtggtggttctatccagcggacccctaagatccaggtgtacagcagacaccccgccgagaacggcaagagcaacttcctgaactgctacgtgtccggctttcaccccagcgacattgaggtggacctgctgaagaacggcgagcggatcgagaaggtggaacacagcgatctgagcttcagcaaggactggtccttctacctgctgtactacaccgagttcacccctaccgagaaggacgagtacgcctgcagagtgaaccacgtgacactgagccagcctaagatcgtgaagtgggatcgcgatatgggcggaggcggatctggtggcggaggaagtggcggcggaggatctggctcccactccttgaagtatttccacacttccgtgtcccggcccggccgcggggagccccgcttcatctctgtgggctacgtggacgacacccagttcgtgcgcttcgacaacgacgccgcgagtccgaggatggtgccgcgggcgccgtggatggagcaggaggggtcagagtattgggaccgggagacacggagcgccagggacaccgcacagattttccgagtgaatctgcggacgctgcgcggctactacaatcagagcgaggccgggtctcacaccctgcagtggatgcatggctgcgagctggggcccgacgggcgcttcctccgcgggtatgaacagttcgcctacgacggcaaggattatctcaccctgaatgaggacctgcgctcctggaccgcggtggacacggcggctcagatctccgagcaaaagtcaaatgatgcctctgaggcggagcaccagagagcctacctggaagacacatgcgtggagtggctccacaaatacctggagaaggggaaggagacgctgcttcacctggagcccccaaagacacacgtgactcaccaccccatctctgaccatgaggccaccctgaggtgctgggccctgggcttctaccctgcggagatcacactgacctggcagcaggatggggagggccatacccaggacacggagctcgtggagaccaggcctgcaggggatggaaccttccagaagtgggcagctgtggtggtgccttctggagaggagcagagatacacgtgccatgtgcagcatgaggggctacccgagcccgtcaccctgagatggaagccggcttcccagcccaccatccccatcgtgggcatcattgctggcctggttctccttggatctgtggtctctggagctgtggttgctgctgtgatatggaggaagaagagctcaggtggaaaaggagggagctactctaaggctgagtggagcgacagtgcccaggggtctgagtctcacagcttgtaatgaagcggccgcgactctagatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgatccagcagagaatggaaagtcaaatttcctgaattgctatgtgtctgggtttcatccatccgacattgaagttgacttactgaagaatggagagagaattgaaaaagtggagcattcagacttgtctttcagcaaggactggtctttctatctcttgtactacactgaattcacccccactgaaaaagatgagtatgcctgccgtgtgaaccatgtgactttgtcacagcccaagatagttaagtggggtaagtcttacattcttttgtaagctgctgaaagttgtgtatgagtagtcatatcataaagctgctttgatataaaaaaggtctatggccatactaccctgaatgagtcccatcccatctgatataaacaatctgcatattgggattgtcagggaatgttcttaaagatcagattagtggcacctgctgagatactgatgcacagcatggtttctgaaccagtagtttccctgcagttgagcagggagcagcagcagcacttgcacaaatacatatacactcttaacacttcttacctactggcttcctctagcttttgtggcagcttcaggtatatttagcactgaacgaacatctcaagaaggtataggcctttgtttgtaagtcctgctgtcctagcatcctataatcctggacttctccagtactttctggctggattggtatctgaggctagtaggaagggcttgttcctgctgggtagctctaaacaatgtattcatgggtaggaacagcagcctattctgccagccttatttctaaccattttagacatttgttagtacatggtattttaaaagtaaaacttaatgtcttccttttttttctccactgtctttttcatagatcgagacatgtaagcagcatcatggaggtaagtttttgaccttgagaaaatgtttttgtttcactgtcctgaggactatttatagacagctctaacatgataaccctcactatgtggagaacattgacagagtaacattttagcagaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 84 - DNA, 1205 bp, Artificial Sequence
  AAVS1 targeting left homology arm
actctgccccaggcctccttaccattccccttcgacctactctcttccgcattggagtcgctttaactggccctggctttggcagcctgtgctgacccatgcagtcctccttaccatccctccctcgacttcccctcttccgatgttgagcccctccagccggtcctggactttgtctccttccctgccctgccctctcctgaacctgagccagctcccatagctcagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccct

SEQ ID NO: 85 - DNA, 1200 bp, Artificial Sequence
  AAVS1 targeting right homology arm
ccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgtacccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctag

SEQ ID NO: 86 - DNA, 7971 bp, Artificial Sequence
  AAVS1 Targeting plasmid
actctgccccaggcctccttaccattccccttcgacctactctcttccgcattggagtcgctttaactggccctggctttggcagcctgtgctgacccatgcagtcctccttaccatccctccctcgacttcccctcttccgatgttgagcccctccagccggtcctggactttgtctccttccctgccctgccctctcctgaacctgagccagctcccatagctcagtctggtctatctgcctggccctggccattgtcactttgcgctgccctcctctcgcccccgagtgcccttgctgtgccgccggaactctgccctctaacgctgccgtctctctcctgagtccggaccactttgagctctactggcttctgcgccgcctctggcccactgtttccccttcccaggcaggtcctgctttctctgacctgcattctctcccctgggcctgtgccgctttctgtctgcagcttgtggcctgggtcacctctacggctggcccagatccttccctgccgcctccttcaggttccgtcttcctccactccctcttccccttgctctctgctgtgttgctgcccaaggatgctctttccggagcacttccttctcggcgctgcaccacgtgatgtcctctgagcggatcctccccgtgtctgggtcctctccgggcatctctcctccctcacccaaccccatgccgtcttcactcgctgggttcccttttccttctccttctggggcctgtgccatctctcgtttcttaggatggccttctccgacggatgtctcccttgcgtcccgcctccccttcttgtaggcctgcatcatcaccgtttttctggacaaccccaaagtaccccgtctccctggctttagccacctctccatcctcttgctttctttgcctggacaccccgttctcctgtggattcgggtcacctctcactcctttcatttgggcagctcccctaccccccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctgcggccgcacgcgtagatctattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgggtcgaggtgagccccacgttctgcttcactctccccatctcccccccctccccacccccaattttgtatttatttattttttaattattttgtgcagcgatgggggcggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgttgccttcgccccgtgccccgctccgcgccgcctcgcgccgcccgccccggctctgactgaccgcgttactcccacaggtgagcgggcgggacggcccttctcctccgggctgtaattagcgcttggtttaatgacggctcgtttcttttctgtggctgcgtgaaagccttaaagggctccgggagggccctttgtgcgggggggagcggctcggggggtgcgtgcgtgtgtgtgtgcgtggggagcgccgcgtgcggcccgcgctgcccggcggctgtgagcgctgcgggcgcggcgcggggctttgtgcgctccgcgtgtgcgcgaggggagcgcggccgggggcggtgccccgcggtgcgggggggctgcgaggggaacaaaggctgcgtgcggggtgtgtgcgtgggggggtgagcagggggtgtgggcgcggcggtcgggctgtaacccccccctgcacccccctccccgagttgctgagcacggcccggcttcgggtgcggggctccgtgcggggcgtggcgcggggctcgccgtgccgggcggggggtggcggcaggtgggggtgccgggcggggcggggccgcctcgggccggggagggctcgggggaggggcgcggcggccccggagcgccggcggctgtcgaggcgcggcgagccgcagccattgccttttatggtaatcgtgcgagagggcgcagggacttcctttgtcccaaatctggcggagccgaaatctgggaggcgccgccgcaccccctctagcgggcgcgggcgaagcggtgcggcgccggcaggaaggaaatgggcggggagggccttcgtgcgtcgccgcgccgccgtccccttctccatctccagcctcggggctgccgcagggggacggctgccttcgggggggacggggcagggcggggttcggcttctggcgtgtgaccggcgggatatctacgaagcggccgccctctgctaaccatgttcatgccttcttctttttcctacagctcctgggcaacgtgctggttattgtgctgtctcatcattttggcaaagtcgacgccaccatgctgctgctggtcacatctctgctgctgtgcgagctgccccatcctgcctttctgctgatccccgacatccagatgacccagaccacaagcagcctgtctgccagcctgggcgatagagtgaccatcagctgtagagccagccaggacatcagcaagtacctgaactggtatcagcaaaagcccgacggcaccgtgaagctgctgatctaccacaccagcagactgcacagcggcgtgccaagcagattttctggcagcggctctggcaccgactacagcctgacaatcagcaacctggaacaagaggatatcgctacctacttctgccagcaaggcaacaccctgccttacacctttggcggaggcaccaagctggaaatcaccggctctacaagcggcagcggcaaacctggatctggcgagggatctaccaagggcgaagtgaaactgcaagagtctggccctggactggtggccccatctcagtctctgagcgtgacctgtacagtcagcggagtgtccctgcctgattacggcgtgtcctggatcagacagcctcctcggaaaggcctggaatggctgggagtgatctggggcagcgagacaacctactacaacagcgccctgaagtcccggctgaccatcatcaaggacaactccaagagccaggtgttcctgaagatgaacagcctgcagaccgacgacaccgccatctactattgcgccaagcactactactacggcggcagctacgccatggattattggggccagggcaccagcgtgaccgtgtctagcacgacgactcctgctccaaggcctcctacacctgcaccaaccattgcaagtcagccgttgagcctccggccagaagcatgtcgcccagccgcaggcggggctgtacacacgagaggcttggatttcgcatgtgacatctatatctgggccccactggccggcacctgcggcgtgctgctgctgagcctggtgatcaccaagcgaggccgcaaaaaactcctttatatattcaagcaaccttttatgaggcccgtccagaccacgcaagaggaagatgggtgctcttgccgctttccagaggaagaggaggggggctgcgaacttagagtgaagttcagcagatccgccgatgctcccgcctatcagcagggccaaaaccagctgtacaacgagctgaacctggggagaagagaagagtacgacgtgctggacaagcggagaggcagagatcctgaaatgggcggcaagcccagacggaagaatcctcaagagggcctgtataatgagctgcagaaagacaagatggccgaggcctacagcgagatcggaatgaagggcgagcgcagaagaggcaagggacacgatggactgtaccagggcctgagcaccgccaccaaggatacctatgatgccctgcacatgcaggccctgcctccaagataataaaacttgtttattgcagcttataatggttacaaataaagcaatagcatcacaaatttcacaaataaagcatttttttcactgcattctagttgtggtttgtccaaactcatcaatgtatcttaccaccccacagtggggccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacgatggagccagagaggatcctgggagggagagcttggcagggggtgggagggaagggggggatgcgtgacctgcccggttctcagtggccaccctgcgctaccctctcccagaacctgagctgctctgacgcggccgtctggtgcgtttcactgatcctggtgctgcagcttccttacacttcccaagaggagaagcagtttggaaaaacaaaatcagaataagttggtcctgagttctaactttggctcttcacctttctagtccccaatttatattgttcctccgtgcgtcagttttacctgtgagataaggccagtagccagccccgtcctggcagggctgtggtgaggaggggggtgtccgtgtggaaaactccctttgtgagaatggtgcgtcctaggtgttcaccaggtcgtggccgcctctactccctttctctttctccatccttctttccttaaagagtccccagtgctatctgggacatattcctccgcccagagcagggtcccgcttccctaaggccctgctctgggcttctgggtttgagtccttggcaagcccaggagaggcgctcaggcttccctgtcccccttcctcgtccaccatctcatgcccctggctctcctgccccttccctacaggggttcctggctctgctcttcagactgagccccgttcccctgcatccccgtacccctgcatcccccttcccctgcatcccccagaggccccaggccacctacttggcctggaccccacgagaggccaccccagccctgtctaccaggctgccttttgggtggattctcctccaactgtggggtgactgcttggcaaactcactcttcggggtatcccaggaggcctggagcattggggtgggctggggttcagagaggagggattcccttctcaggttacgtggccaagaagcaggggagctgggtttgggtcaggtctgggtgtggggtgaccagcttatgctgtttgcccaggacagcctagaggctaggtggaggctcagtgatgataagtctgcgatggtggatgcatgtgtcatggtcatagctgtttcctgtgtgaaattgttatccgctcagagggcacaatcctattccgcgctatccgacaatctccaagacattaggtggagttcagttcggcgtatggcatatgtcgctggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctctattcaacaaagccgccgtcccgtcaagtcagcgtaaatgggtagggggcttcaaatcgtcctcgtgataccaattcggagcctgcttttttgtacaaacttgttgataatggcaattcaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagagccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccagatacctgaaacaaaacccatcgtacggccaaggaagtctccaataactgtgatccaccacaagcgccagggttttcccagtcacgacgttgtaaaacgacggccagtcatgcataatccgcacgcatctggaataaggaagtgccattccgcctgacct

SEQ ID NO: 87 - protein, 21 aa, Artificial Sequence
  T2A variant
GSGEGRGSLLTCGDVEENPGP

SEQ ID NO: 88 - DNA, 63 bp, Artificial Sequence
  T2A variant
ggcagcggcgaaggcagaggatctctgctgacatgtggcgacgtggaagagaaccccggacct

SEQ ID NO: 89 - protein, 376 aa, Artificial Sequence
  HSV thymidine kinase A168H variant
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEAN

SEQ ID NO: 90 - DNA, 1128 bp, Artificial Sequence
  HSV thymidine kinase A168H variant
atggccagctatccttgtcaccagcacgccagcgcctttgatcaggccgcaagatctagaggccacagcaacagaagaacagccctgcggcctcggagacagcaagaggctacagaagttcggctggaacagaagatgcccacactgctgcgggtgtacatcgatggccctcacggcatgggcaagaccaccacaacacagctgctggtggccctgggcagcagagatgatatcgtgtacgtgcccgagcctatgacctactggcaggttctgggagccagcgagacaatcgccaacatctacaccacacagcaccggctggatcagggcgaaatttctgctggcgacgccgccgtggttatgacatctgcccagatcaccatgggcatgccttacgccgtgacagatgctgtgctggcccctcacattggcggagaagccggatcttctcatgcccctccaccagctctgaccctgatcttcgacagacaccctatcgcccatctgctgtgttatcctgccgccagatacctgatgggcagcatgacacctcaggccgtgctggctttcgtggccctgattcctcctacactgcccggcaccaatatcgtgctgggagccctgcctgaggaccggcacattgatagactggccaagagacagcggcctggcgagagactggatctggctatgctggccgccatcagaagagtgtacggcctgctggccaacaccgtgcggtatcttcaaggcggcggatcttggagagaggactggggacaactgagcggcacagcagttcctccacaaggcgctgagcctcagtctaacgctggacccagacctcacatcggcgacaccctgtttaccctgttcagagcccctgagctgctggctcctaacggcgacctgtacaacgtgttcgcctgggctcttgacgtgctggcaaaaagactgcggcccatgcacgtgttcatcctggactacgatcagtcccctgccggctgtagagatgctctgctgcagctgacaagcggcatggtgcagacccacgttacaacccctggcagcatccccaccatctgtgacctggccagaaccttcgccagagagatgggagaagccaac

SEQ ID NO: 91 - protein, 61 aa, Artificial Sequence
  CD52
MKRFLFLLLTISLLVMVQIQTGLSGQNDTSQTSSPSASSNISGGIFLFFVANAIIHLFCFS

SEQ ID NO: 92 - DNA, 186 bp, Artificial Sequence
  CD52
atgaagaggttcctgttcctgctgctgaccatcagcctgctggtgatggtgcagatccagaccggcctgagcggccagaacgacaccagccagaccagcagccccagcgccagcagcaacatcagcggcggcatcttcctgttcttcgtggccaacgccatcatccacctgttctgcttcagctga

SEQ ID NO: 93 - protein, 1138 aa, Artificial Sequence
  WT HSV-TK-T2A-PSMA (N9del)
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSGEGRGSLLTCGDVEENPGPMWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 94 - protein, 1138 aa, Artificial Sequence
  (A168H) HSV-TK-T2A-PSMA (N9del)
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSGEGRGSLLTCGDVEENPGPMWNLLARRPRWLCAGALVLAGGFFLLGFLFGWFIKSSNEATNITPKHNMKAFLDELKAENIKKFLYNFTQIPHLAGTEQNFQLAKQIQSQWKEFGLDSVELAHYDVLLSYPNKTHPNYISIINEDGNEIFNTSLFEPPPPGYENVSDIVPPFSAFSPQGMPEGDLVYVNYARTEDFFKLERDMKINCSGKIVIARYGKVFRGNKVKNAQLAGAKGVILYSDPADYFAPGVKSYPDGWNLPGGGVQRGNILNLNGAGDPLTPGYPANEYAYRRGIAEAVGLPSIPVHPIGYYDAQKLLEKMGGSAPPDSSWRGSLKVPYNVGPGFTGNFSTQKVKMHIHSTNEVTRIYNVIGTLRGAVEPDRYVILGGHRDSWVFGGIDPQSGAAVVHEIVRSFGTLKKEGWRPRRTILFASWDAEEFGLLGSTEWAEENSRLLQERGVAYINADSSIEGNYTLRVDCTPLMYSLVHNLTKELKSPDEGFEGKSLYESWTKKSPSPEFSGMPRISKLGSGNDFEVFFQRLGIASGRARYTKNWETNKFSGYPLYHSVYETYELVEKFYDPMFKYHLTVAQVRGGMVFELANSIVLPFDCRDYAVVLRKYADKIYSISMKHPQEMKTYSVSFDSLFSAVKNFTEIASKFSERLQDFDKSNPIVLRMMNDQLMFLERAFIDPLGLPDRPFYRHVIYAPSSHNKYAGESFPGIYDALFDIESKVDPSKAWGEVKRQIYVAAFTVQAAAETLSEVA

SEQ ID NO: 95 - DNA, 3417 bp, Artificial Sequence
  (A168H) HSV-TK-T2A-PSMA (N9del)
atggccagctatccttgtcaccagcacgccagcgcctttgatcaggccgcaagatctagaggccacagcaacagaagaacagccctgcggcctcggagacagcaagaggctacagaagttcggctggaacagaagatgcccacactgctgcgggtgtacatcgatggccctcacggcatgggcaagaccaccacaacacagctgctggtggccctgggcagcagagatgatatcgtgtacgtgcccgagcctatgacctactggcaggttctgggagccagcgagacaatcgccaacatctacaccacacagcaccggctggatcagggcgaaatttctgctggcgacgccgccgtggttatgacatctgcccagatcaccatgggcatgccttacgccgtgacagatgctgtgctggcccctcacattggcggagaagccggatcttctcatgcccctccaccagctctgaccctgatcttcgacagacaccctatcgcccatctgctgtgttatcctgccgccagatacctgatgggcagcatgacacctcaggccgtgctggctttcgtggccctgattcctcctacactgcccggcaccaatatcgtgctgggagccctgcctgaggaccggcacattgatagactggccaagagacagcggcctggcgagagactggatctggctatgctggccgccatcagaagagtgtacggcctgctggccaacaccgtgcggtatcttcaaggcggcggatcttggagagaggactggggacaactgagcggcacagcagttcctccacaaggcgctgagcctcagtctaacgctggacccagacctcacatcggcgacaccctgtttaccctgttcagagcccctgagctgctggctcctaacggcgacctgtacaacgtgttcgcctgggctcttgacgtgctggcaaaaagactgcggcccatgcacgtgttcatcctggactacgatcagtcccctgccggctgtagagatgctctgctgcagctgacaagcggcatggtgcagacccacgttacaacccctggcagcatccccaccatctgtgacctggccagaaccttcgccagagagatgggagaagccaacggcagcggcgaaggcagaggatctctgctgacatgtggcgacgtggaagagaaccccggacctatgtggaacctgctggctagacggcccagatggctttgtgctggtgctctggttctggctggcggctttttcctgctgggcttcctgtttggctggttcatcaagagcagcaacgaggccaccaacatcacccctaagcacaacatgaaggcctttctggacgagctgaaggccgagaatatcaagaagttcctctacaacttcacgcagatccctcacctggccggcaccgagcagaattttcagctggccaagcagatccagagccagtggaaagagttcggcctggactctgtggaactggcccactacgatgtgctgctgagctaccccaacaagacacaccccaactacatcagcatcatcaacgaggacggcaacgagatcttcaacaccagcctgttcgagcctccacctcctggctacgagaacgtgtccgatatcgtgcctccattcagcgctttcagccctcaagggatgcctgagggcgatctggtgtacgtgaactacgccagaaccgaggacttcttcaagctggaacgggacatgaagatcaactgctccggcaagatcgtgatcgcccgctacggcaaagtgttccggggcaacaaagtgaagaacgcccagctggcaggcgccaaaggcgtgatcctgtatagcgaccccgccgactattttgcccctggcgtgaagtcttaccccgacggctggaatcttcctggtggcggagtgcagagaggcaacatcctgaaccttaacggcgcaggcgaccctctgacacctggctatcctgccaatgagtacgcctacagacggggaattgccgaggctgtgggactgccttctatccctgtgcaccccatcggctactacgacgcccagaaactgctggaaaagatgggcggaagcgcccctcctgactctagttggagaggctctctgaaggtgccctacaatgtcggcccaggcttcaccggcaacttcagcacccagaaagtgaaaatgcacatccacagcaccaacgaagtgacccggatctacaacgtgatcggcacactgagaggcgccgtggaacccgacagatatgtgatcctcggcggccacagagatagctgggtgttcggaggaatcgaccctcagtctggtgccgctgtggtgcacgaaatcgtgcggtcttttggcaccctgaagaaagaaggctggcgccccagacggaccatcctgtttgcctcttgggacgccgaggaattcggactgctgggatctacagagtgggccgaagagaacagcagactgctgcaagaaagaggcgtggcctacatcaacgccgacagcagcatcgagggcaactacaccctgagagtggactgcacccctctgatgtacagcctggtgcacaacctgaccaaagagctgaagtcccctgacgagggctttgagggcaagagcctgtacgagagctggaccaagaagtccccatctcctgagttcagcggcatgccccggatctctaagctcggctctggcaacgacttcgaggtgttcttccagcggctgggaatcgcttctggcagagccagatacaccaagaactgggagacaaacaagttctccggctatcccctgtaccacagcgtgtacgagacatacgagctggtcgagaagttctacgaccccatgttcaagtaccacctgacagtggcccaagtgcgcggaggcatggtgttcgaactggccaatagcatcgtgctgcccttcgactgcagagactatgccgtggtgctgcggaagtacgccgataagatctacagcatcagcatgaagcacccgcaagagatgaagacctacagcgtgtccttcgatagcctgttcagcgccgtgaagaacttcaccgagatcgccagcaagttcagcgagcggctgcaggacttcgacaagagcaacccaatcgtgctgaggatgatgaacgaccagctgatgttcctggaacgcgccttcatcgacccactgggcttgcccgatagacccttctaccggcacgtgatctatgcccctagcagccacaacaaatacgccggcgagagcttccccggcatctacgatgccctgttcgacatcgagagcaaggtggacccatctaaggcctggggcgaagtgaagcggcagatctacgtggccgcattcacagtgcaggctgccgccgaaacactgtctgaagtggcttga

SEQ ID NO: 96 - protein, 458 aa, Artificial Sequence
  WT HSV-TK-T2A-CD52
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAALLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSGEGRGSLLTCGDVEENPGPMKRFLFLLLTISLLVMVQIQTGLSGQNDTSQTSSPSASSNISGGIFLFFVANAIIHLFCFS

SEQ ID NO: 97 - protein, 458 aa, Artificial Sequence
  (A168H) HSV-TK-T2A-CD52
MASYPCHQHASAFDQAARSRGHSNRRTALRPRRQQEATEVRLEQKMPTLLRVYIDGPHGMGKTTTTQLLVALGSRDDIVYVPEPMTYWQVLGASETIANIYTTQHRLDQGEISAGDAAVVMTSAQITMGMPYAVTDAVLAPHIGGEAGSSHAPPPALTLIFDRHPIAHLLCYPAARYLMGSMTPQAVLAFVALIPPTLPGTNIVLGALPEDRHIDRLAKRQRPGERLDLAMLAAIRRVYGLLANTVRYLQGGGSWREDWGQLSGTAVPPQGAEPQSNAGPRPHIGDTLFTLFRAPELLAPNGDLYNVFAWALDVLAKRLRPMHVFILDYDQSPAGCRDALLQLTSGMVQTHVTTPGSIPTICDLARTFAREMGEANGSGEGRGSLLTCGDVEENPGPMKRFLFLLLTISLLVMVQIQTGLSGQNDTSQTSSPSASSNISGGIFLFFVANAIIHLFCFS

SEQ ID NO: 98 - DNA, 1377 bp, Artificial Sequence
  (A168H) HSV-TK-T2A-CD52
atggccagctatccttgtcaccagcacgccagcgcctttgatcaggccgcaagatctagaggccacagcaacagaagaacagccctgcggcctcggagacagcaagaggctacagaagttcggctggaacagaagatgcccacactgctgcgggtgtacatcgatggccctcacggcatgggcaagaccaccacaacacagctgctggtggccctgggcagcagagatgatatcgtgtacgtgcccgagcctatgacctactggcaggttctgggagccagcgagacaatcgccaacatctacaccacacagcaccggctggatcagggcgaaatttctgctggcgacgccgccgtggttatgacatctgcccagatcaccatgggcatgccttacgccgtgacagatgctgtgctggcccctcacattggcggagaagccggatcttctcatgcccctccaccagctctgaccctgatcttcgacagacaccctatcgcccatctgctgtgttatcctgccgccagatacctgatgggcagcatgacacctcaggccgtgctggctttcgtggccctgattcctcctacactgcccggcaccaatatcgtgctgggagccctgcctgaggaccggcacattgatagactggccaagagacagcggcctggcgagagactggatctggctatgctggccgccatcagaagagtgtacggcctgctggccaacaccgtgcggtatcttcaaggcggcggatcttggagagaggactggggacaactgagcggcacagcagttcctccacaaggcgctgagcctcagtctaacgctggacccagacctcacatcggcgacaccctgtttaccctgttcagagcccctgagctgctggctcctaacggcgacctgtacaacgtgttcgcctgggctcttgacgtgctggcaaaaagactgcggcccatgcacgtgttcatcctggactacgatcagtcccctgccggctgtagagatgctctgctgcagctgacaagcggcatggtgcagacccacgttacaacccctggcagcatccccaccatctgtgacctggccagaaccttcgccagagagatgggagaagccaacggcagcggcgaaggcagaggatctctgctgacatgtggcgacgtggaagagaaccccggacctatgaagaggttcctgttcctgctgctgaccatcagcctgctggtgatggtgcagatccagaccggcctgagcggccagaacgacaccagccagaccagcagccccagcgccagcagcaacatcagcggcggcatcttcctgttcttcgtggccaacgccatcatccacctgttctgcttcagctga